SEQUENCE LISTING (9 sequences)

SEQ ID NO: 1 - protein, 21 aa, Unknown
  Plasmid sequence
YSGRGKGGKGLGKGGAKRHRK

SEQ ID NO: 2 - protein, 73 aa, Homo sapiens
FAWPFRQPVDAVKLGLPDYHKIIKQPMDMGTIKRRLENNYYWAASECMQDFNTMFTNCYIYNKPTDDIVLMAQ

SEQ ID NO: 3 - protein, 73 aa, Homo sapiens
FAWPFYQPVDAIKLNLPDYHKIIKNPMDMGTIKKRLENNYYWSASECMQDFNTMFTNCYIYNKPTDDIVLMAQ

SEQ ID NO: 4 - protein, 73 aa, Homo sapiens
FAWPFQQPVDAVKLNLPDYYKIIKTPMDMGTIKKRLENNYYWNAQECIQDFNTMFTNCYIYNKPGDDIVLMAE

SEQ ID NO: 5 - protein, 73 aa, Homo sapiens
FSWPFQRPVDAVKLQLPDYYTIIKNPMDLNTIKKRLENKYYAKASECIEDFNTMFSNCYLYNKPGDDIVLMAQ

SEQ ID NO: 6 - protein, 73 aa, Homo sapiens
YAWPFYKPVDASALGLHDYHDIIKHPMDLSTVKRKMENRDYRDAQEFAADVRLMFSNCYKYNPPDHDVVAMAR

SEQ ID NO: 7 - protein, 73 aa, Homo sapiens
YAWPFYKPVDAEALELHDYHDIIKHPMDLSTVKRKMDGREYPDAQGFAADVRLMFSNCYKYNPPDHEVVAMAR

SEQ ID NO: 8 - protein, 73 aa, Homo sapiens
YAWPFYKPVDVEALGLHDYCDIIKHPMDMSTIKSKLEAREYRDAQEFGADVRLMFSNCYKYNPPDHEVVAMAR

SEQ ID NO: 9 - protein, 73 aa, Homo sapiens
YAWPFYNPVDVNALGLHNYYDVVKNPMDLGTIKEKMDNQEYKDAYKFAADVRLMFMNCYKYNPPDHEVVTMAR

(19) United States
(12) Patent Application Publication
(10) Pub. No.: US 2017/0122958 A1
(43) Pub. Date: May 4, 2017

(54) Enzymes Functional Probes

(71) Applicants: University of Dundee; Cambridge Enterprise Limited

(72) Inventors: Enrique Lin Shiao, Philadelphia, PA (US); Matthias Gerard Jacky Baud, Cambridge (GB); Alessio Ciulli, Dundee (GB); Kwok-Ho Chan, Dundee (GB); Michael Zengerle, Dundee (GB)

(21) Appl. No.: 15/039,350

(22) PCT Filed: Nov. 28, 2014
(86) PCT No.: PCT/GB2014/053549

§ 371 Date: May 25, 2016

(30) Foreign Application Priority Data
Nov. 28, 2013 (GB) 1320994.5
Jan. 21, 2014 (GB) 1401001.1

(51) Int. Cl. G01N 33/68; C07D 487/04; C12N 15/10

(52) U.S. Cl. CPC G01N 33/6803; C12N 15/102; C07D 487/04; G01N 2500/04

(57) ABSTRACT

A method of selectively inhibiting a bromodomain in the presence of other bromodomains comprising introducing a functionally silent mutation into the bromodomain in the presence of other wild type bromodomains and selectively inhibiting the mutated bromodomain.

### FIELD OF THE INVENTION

The present invention relates to methods of inhibiting single bromodomains and the use of such methods to identify the biological function of bromodomains. The invention also relates to compounds capable of inhibiting single bromodomains.

### BACKGROUND OF THE INVENTION

Histones are highly conserved proteins found in eukaryotic cell nuclei that are responsible for packaging and ordering DNA into high order structural units. The histones act as spools around which DNA strands wind to form the nucleosomes. The resulting structure resembles beads on a string and is referred to as primary chromatin. The primary chromatin is then subject to further compaction and organisation, resulting in higher order chromatin structures. Each human cell contains approximately 1.8 metres of DNA, which is packaged by the histones into approximately 90 micrometres of chromatin.

The structure of chromatin is not fixed and varies depending on the cell's progress through the cell cycle. As the cell prepares to divide, the chromatin is packaged more tightly to assist with chromosome separation during anaphase. Conversely, during interphase, the chromatin is relatively loosely packed to allow access to the DNA and RNA polymerases responsible for replication and transcription of the DNA. The transcription of sections of the DNA into the chemically related RNA is the first step in gene expression.

Changes in chromatin structure are mediated by DNA methylation, ATP dependent chromatin remodelers, histone variants and histone modifications. Histone modifications play a fundamental role in this process. A number of such modifications have been identified, primarily at the N terminal ends of histones H3 and H4. These N terminal ends form long tails that protrude from the nucleosomes and which can be accessed by a number of different enzymes. Known modifications of the histone tails include acetylation, methylation, phosphorylation, ubiquitination, SUMOylation, ADP ribosylation and citrullination. It is thought that these modifications form a distinct ‘histone code’ although only a few specific modifications have been studied in any detail, of which the majority are involved in DNA transcription.

Histone modifications are epigenetic, that is they are functional modifications to the genome that do not involve changes to the underlying DNA sequence. They serve to encode an additional layer of information for regulating and controlling gene expression. Although histone modifications are covalent, they are known to be reversible and their activity is highly regulated by a distinct set of proteins known as writers, erasers and readers of the epigenome.

Bromodomains, known as readers of the epigenome are functional protein domains, found in a large number of proteins, which recognise and bind to the histone tails by identifying acetylated lysine residues on them. Around 61 distinct human bromodomains have been identified and 46 proteins containing up to six bromodomains each have been identified in the human genome, for example, the Bromo and Extra-Terminal (BET) proteins, Brd2, Brd3, Brd4 and the testis-specific BrdT, which play a key role in the epigenetic regulation of gene expression. There is currently a great deal of interest in identifying compounds that can inhibit or otherwise affect the function of BET proteins, as this opens up the possibility of using small molecules to modulate gene expression. This would be a powerful research tool for studying gene function and offers the potential for developing new treatments which avoid the ethical and practical difficulties associated with conventional gene therapies. Misregulation of BET protein activity has been found to be involved in various disease states, notably in cancer and inflammation.

A number of bromodomain inhibitors are currently in clinical trials.

Resverlogix Inc. have a lead compound RVX-208 in phase 2 clinical trials for the treatment of atherosclerosis. RVX-208 has been found to increase transcription of the ApoA-1 gene resulting in the production of more ApoA-1 and high density lipoprotein (HDL).

Compound OTX015 developed by Mitsubishi and licensed to Oncoethix, is currently in phase 1 clinical trials for the treatment of acute leukemia and other haematological cancers.

Researchers at Constellation Pharmaceuticals have reported an isoxazole-based BET bromodomain inhibitor, again binding with high affinity to the acetyl-lysine (KAc) pocket. Constellation Pharmaceuticals currently have a compound (CPI-0610) in phase 1 clinical trials for patients with aggressive lymphoma.

GSK compound iBET762 is currently in Phase I clinical trials for nut midline carcinoma (NMC), a rare but lethal form of lung cancer arising from a genetic translocation.

Other cell-permeable small molecules based on a thienotriazolodiazepine scaffold, such as iBET762 (GSK, from now called iBET for convenience), JQ1 (Mitsubishi, Structural Genomics Consortium Oxford in collaboration with Harvard University) and GW841819X (GSK) were shown to bind with high affinity to the KAc binding pocket of BET bromodomains (Kd 50-300 nM). These inhibitors of the bromodomain-histone interaction have shown considerable promise as potential therapeutic agents against various cancers. For example, they display activity in vivo against NUT midline carcinoma [1], multiple myeloma [2], mixed-lineage leukemia [3], and acute myeloid leukemia [4].

WO2011054553 and WO2011054845 disclose bromodomain inhibitors based on diazepine scaffolds.

Other inhibitors based on a quinazolinone have also been developed, for example compound PFI-1 [5] from the Structural Genomics Consortium (SGC) in Oxford in collaboration with Pfizer.

These compounds are pan-selective for the eight BET subfamily members (Brd2(1), Brd3(1), Brd4(1), BrdT(1), Brd2(2), Brd2(2), Brd3(2) and BrdT(2)) relative to other human bromodomains, however, due to the high conservation of the KAc binding sites, they exhibit poor selectivity for individual BET bromodomains. This inherent lack of target selectivity limits their use as chemical genetic tools that would allow elucidation of the role of individual BET bromodomains or individual BET proteins, and their further validation as drug targets in disease conditions.

Many bromodomains have unknown or unclear functions and it would therefore be advantageous to have the ability to selectively modulate the activity of a given bromodomain containing protein in order to examine its effect on the cell. However, as mentioned above, since bromodomains tend to be very similar from one protein to the next, selectively inhibiting the function of one specific protein or the function of one specific bromodomain within a protein having a number of such domains is technically challenging.

As such it is a challenging problem both to identify the biological function of particular BET proteins and to develop suitably selective inhibitors of those proteins for therapeutic use. Accordingly, there is an on-going need in the art for new technologies and methods to investigate BET bromodomain function with controlled selectivity and thereby validating them as drug target in a range of disease conditions.

It is an object of the present invention to obviate or mitigate one or more of the abovementioned problems.

### SUMMARY OF THE INVENTION

The present invention is based in part on studies by the inventors into methods of selectively targeting a single bromodomain or bromodomain type in the presence of other bromodomains.

According to the invention, we provide a method of selectively inhibiting one mutant bromodomain in the presence of a plurality of other wild type bromodomain.

According to a first aspect of the invention, there is provided a method of selectively inhibiting a bromodomain in a protein in the presence of a plurality of other wild type bromodomains, the method comprising the steps of introducing a functionally silent mutation into a bromodomain in a protein in the presence of a plurality of other wild type bromodomains and selectively inhibiting the mutated bromodomain.

According to the invention, we also provide a method of identifying the physiological function of a bromodomain in a protein by; introducing a functionally silent mutation into one bromodomain in the presence of a plurality of other wild type bromodomains, selectively inhibiting the mutant bromodomain and evaluating the effect of the inhibited protein.

According to a second aspect of the invention, there is provided a method of identifying the physiological function of a bromodomain in a protein, the method comprising the steps of introducing a functionally silent mutation into one bromodomain in a protein in the presence of a plurality of other wild type bromodomains, selectively inhibiting the mutated bromodomain and evaluating the effect of the inhibition.

The method of identifying the physiological function of the bromodomain in a protein may be used as a screening method to identify inhibitors of a physiological function of a bromodomain. Therefore, according to a further aspect of the present invention there is provided a screening method comprising the steps of introducing a functionally silent mutation into one bromodomain in a protein in the presence of a plurality of other wild type bromodomains, attempting to selectively inhibit a physiological function of the mutated bromodomain using a test inhibitor, and determining whether the physiological function of the bromodomain has been inhibited. The invention may, therefore, provide an inhibitor obtainable by the process of aforementioned screening method. The inhibitor may be a compound, such as small molecule with a molecular weight of less than 1 kDa for example.

The inventors have observed that it is possible to inhibit a specific bromodomain within a protein by introducing a mutation into that bromodomain and then specifically targeting the mutated bromodomain for inhibition. Such an approach allows the function of individual bromodomains to be elucidated. The skilled person will appreciate that the use of the methods described above could be used to inhibit multiple bromodomains of the same bromodomain type.

In one embodiment, the step of selectively inhibiting the mutated bromodomain includes addition of a compound which specifically binds the mutated bromodomain, such as small molecule with a molecular weight of less than 1 kDa for example.

The inventors have shown that if a specific mutation is introduced into a bromodomain, compounds which bind specifically to that bromodomain (and with significantly less affinity to a wild type, non-mutated bromodomain) can be generated. The use of such compounds specifically disrupt the interaction of said mutant bromodomain, with less disrupting activity towards a wild type, non-mutated bromodomain. Suitable compounds which can be used to bind selectively to mutated bromodomains are discussed further below.

In an embodiment of the invention, the protein may be a bromo and extra-terminal (BET) protein. The protein may be selected from Brd2(1), Brd2(2), Brd3(1), Brd3(2), Brd4(1), Brd4(2), Brdt(1) and Brdt(2). In a particular embodiment, the protein is Brd2(1), Brd2(2), Brd4(1) or Brd4(2).

Advantageously the mutation may be created by site specific mutagenesis. The functionally silent mutation may be introduced by site directed mutagenesis.

Techniques used for genetic modification will be known to a person skilled in the art, but for reference see Sambrook & Russell, Molecular Cloning: A Laboratory Manual (3^{rd} edition).

The term “functionally silent” as used herein means that the mutation introduced does not substantially affect the function of the bromodomain (e.g. its ability to bind acetylated lysine residues). The skilled person will appreciate that the introduction of a mutation may result in some functional alterations, such as a reduced affinity for acetylated lysine residues. However, the mutation should not render the bromodomain non-functional. For example, the mutated bromodomain may retain over 95%, 90%, 80%, 70%, 60% or 50% of the wild type functionality.

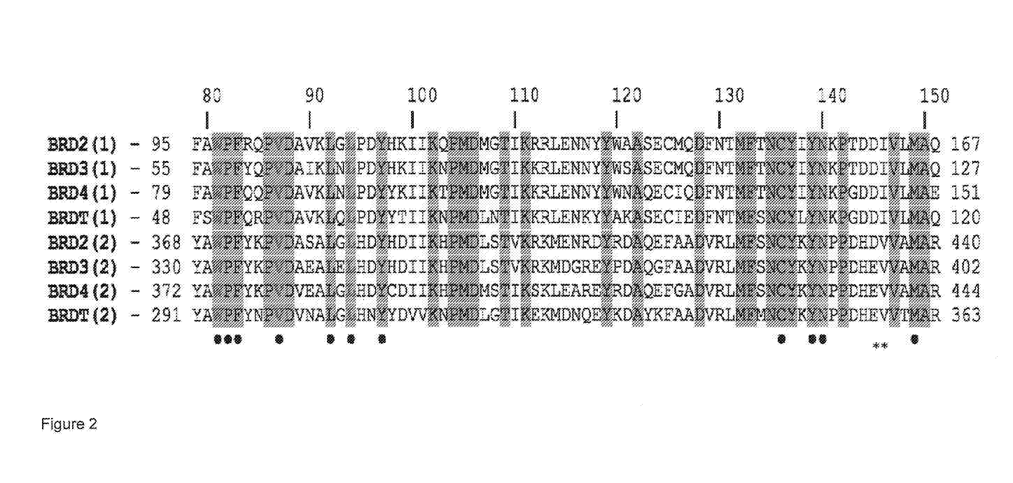

Preferably the amino acid being replaced is a conserved amino acid. The functionally silent mutation may be introduced at an amino acid position which is conserved between bromodomains. The phrase “conserved between bromodomains” as used herein refers to specific amino acids which are evolutionarily conserved in a bromodomain subfamily. For example, FIG. 2 shows a sequence alignment of the eight BET bromodomains. Residues which are conserved throughout the BET bromodomain subfamily are highlighted.

Preferably the amino acid being replaced is selected from Tryptophan 81, Valine 87, Leucine 94 or Methionine 149 in Brd4(1) or, in other bromodomain containing proteins, a conserved equivalent thereof. Preferably the amino acid being replaced is Leucine 94 or Methionine 149.

The functionally silent mutation may be introduced at a conserved position equivalent to Trp81, Val87, Leu94 or Met149 in Brd4(1). For example, Leu94 in Brd4(1) corresponds to Leu70 in Brd3(1), Leu110 in Brd2(1), Leu63 in Brdt(1) (see FIG. 2). Preferably, the functionally silent mutation is introduced at a conserved position equivalent to Leu94 or Met149 in Brd4(1).

In one embodiment, the functionally silent mutation is generated by replacement of an amino acid with alanine, valine or isoleucine, preferably alanine.

The protein may comprise a plurality of bromodomains. Advantageously the functionally silent mutation is introduced into a single one of the said plurality of bromodomains.

Advantageously, inhibition of the mutant protein is at least 30 fold greater than that of the wild type protein. In an embodiment of the invention, inhibition of the mutated bromodomain is at least 30 fold greater than inhibition of the wild type bromodomain. The term “wild type” as used herein means a bromodomain which retains the wild type residue in the position otherwise mutated in the approach e.g. Leu94 in Brd4(1). However, the skilled person would appreciate that the entire protein need not be wild type and that other mutations which do not significantly affect the binding of the bromodomain to acetylated lysine residues may be present in the protein.

According to the invention, we provide compounds of Formulae (I), (II), (Ill), (IV), (V), (VI), (VII) and (VIII).

In a third aspect of the invention there is provided, a compound for use in inhibiting a bromodomain, wherein the compound has the formula (I):

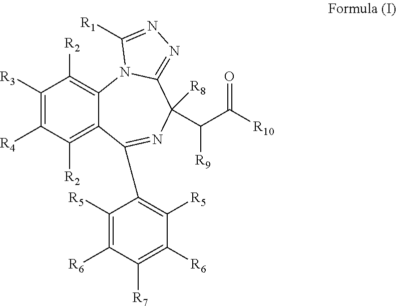

Each one of R₁, R₂, R₃, R₄ and R₈ are independently: hydrogen, a C1-6 linear, branched or substituted alkyl, alkenyl, alkynyl or alkoxy group. Each one of R₅, R₆ and R₇ are independently: hydrogen, halogen, NR₁₁R₁₂ or a C1-6 linear, branched or substituted alkyl, alkenyl, alkynyl group. Any two of R₄, R₅ and R₆, together with the atoms to which they are attached may be joined to form an optionally substituted C1-6 cycloalkyl, heterocyclic, aromatic or heteroaromatic moiety. R₁₁ and R₁₂ are independently hydrogen or C1-6 linear, branched or substituted alkyl, alkenyl, alkynyl group. R₉ is hydrogen, or C1-6 linear or branched alkyl, alkenyl or alkynyl, optionally substituted by one or more amine or hydroxy groups. R₁₀ is R₁₃, OR₁₃, NHR₁₃ or NR₁₃R₁₃, or an optionally substituted C1-6 cycloalkyl, heterocyclic, aromatic or heteroaromatic moiety and R₁₃ is a C1-6 linear, or branched alkyl, alkenyl or alkynyl group. When R₄ is methoxy, at least one of R₂, R₃, R₅, R₆, R₈ or R₉ may not be hydrogen.

In a preferred embodiment the compound has the formula (II):

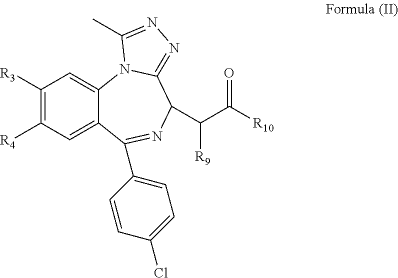

One or both of R₃ and R₄ are alkoxy groups. In an embodiment, the alkoxy groups are methoxy groups.

R₉ is hydrogen, or C1-6 linear or branched alkyl, alkenyl or alkynyl, optionally substituted by one or more amine or hydroxy groups. R₁₀ is R₁₃, OR₁₃, NHR₁₃ or NR₁₃R₁₃, or an optionally substituted C1-6 cycloalkyl, heterocyclic, aromatic or heteroaromatic moiety. R₁₃ is a C1-6 linear or branched alkyl, alkenyl or alkynyl group.

In one embodiment, the compound has the formula (III):

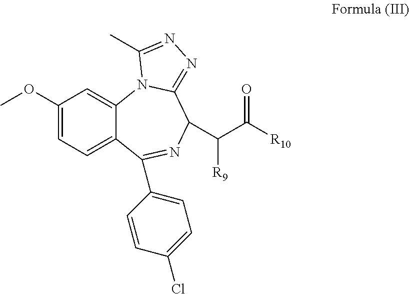

In an alternative embodiment, the compound has the formula (IV):

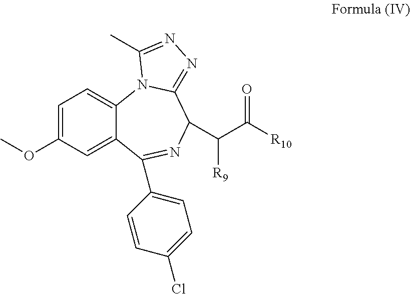

In these embodiments, R₉ is hydrogen, or C1-6 linear or branched alkyl, alkenyl or alkynyl, optionally substituted by one or more amine or hydroxy groups. R₁₀ is R₁₃, OR₁₃, NHR₁₃ or NR₁₃R₁₃, or an optionally substituted C1-6 cycloalkyl, heterocyclic, aromatic or heteroaromatic moiety. R₁₃ is a C1-6 linear or branched alkyl, alkenyl or alkynyl group.

In an embodiment, R₉ may be a C1-4 linear, branched or cycloalkyl group and R₁₀ may be OR₁₃, wherein R₁₃ is a C1-6 linear or branched alkyl, alkenyl or alkynyl group.

In an embodiment R₁₃ is a C1-6 linear or branched alkyl. Preferably R₁₃ is a linear alkyl.

In one embodiment the compound has the formula (V):

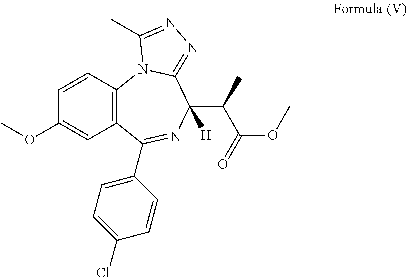

In an alternative embodiment the compound has the formula (VI):

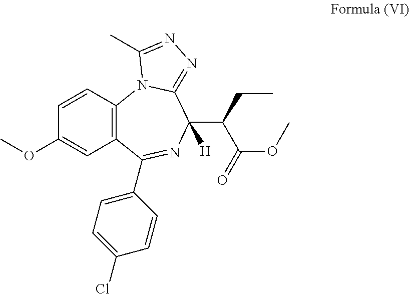

According to a fourth aspect of the invention there is provided a compound for use in inhibiting a bromodomain, wherein the compound has the formula (VII):

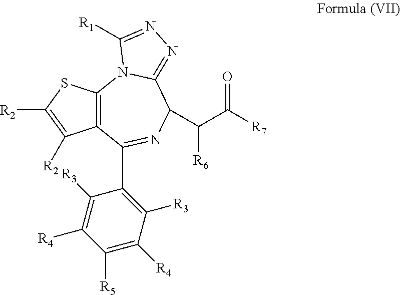

R₁, R₂, R₃, R₄ and R₅ are independently hydrogen, a halogen or a C1-6 linear, branched or substituted alkyl, alkenyl or alkynyl group. R₆ is a C1-6 linear or branched alkyl, alkenyl or alkynyl group, optionally substituted by one or more amine or hydroxy groups. R₇ is OH, OR₈, NHR₈ or NR₈R₉ and R₈ and R₉ is a C1-6 linear, branched or substituted alkyl, alkenyl or alkynyl group.

In one embodiment, R₈ and R₉, together with the atom to which they are attached are fused to form a C1-6, heterocyclic, heteroaromatic, substituted heterocyclic or substituted heteroaromatic ring.

In one embodiment, R₂ is a methyl group.

In one embodiment, R₇ is OR₈. Preferably R₈ is methyl or tertiary-butyl (t-butyl).

In an alternative embodiment R₇ is NHR₈. Preferably R₈ is ethyl.

According to a fifth aspect of the invention there is provided a compound for use in inhibiting a bromodomain, wherein the compound has the formula (VIII):

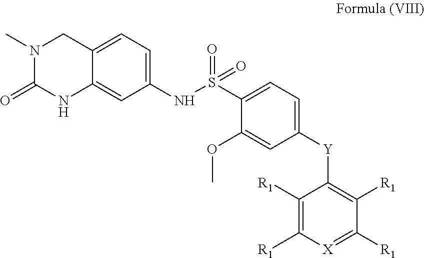

X may be C or N.

Y may be C, C═O, O, S, SO₂ or NH.

Each R₁ may independently be C1-6 linear or branched alkyl, alkoxy or a halogen.

Preferably Y is C═O.

According to a further aspect of the invention there is provided a compound according to the third, fourth or fifth aspects of the invention for use as a medicament, for example in diseases such as cancer and inflammatory disease.

According to a further aspect of the invention there is provided a composition comprising a compound according to the third, fourth or fifth aspects of the invention.

According to a yet further aspect of the invention there is provided a composition for use as a medicament comprising a compound according to the third, fourth or fifth aspects of the invention.

According to a further aspect of the invention there is provided a method according to the first or second aspects of the invention wherein the step of selectively inhibiting the mutated bromodomain includes using a compound according to the third, fourth or fifth aspects of the invention.

According to the invention we provide compounds of Formulae I, II, III, IV, V, VI, VII or VIII for use in the inhibition of one mutant bromodomain in the presence of a plurality of other wild type bromodomains.

According to the invention we provide a method of identifying the physiological function of a bromodomain in a protein by; introducing a functionally silent mutation into one bromodomain in the presence of a plurality of other wild-type bromodomains, selectively inhibiting the mutant bromodomain using a compound of Formulae I, II, III, IV, V, VI, VII or VIII and evaluating the effect of the inhibited protein.

As bromodomains are known to be involved in the control of gene expression, there is a great deal of interest in identifying compounds that can inhibit or otherwise affect the function of bromodomains. This opens up the possibility of using small molecules to modulate gene expression which would be a powerful research tool for studying gene function and offers the potential for developing new treatments which avoid the ethical and practical difficulties associated with conventional gene therapies.

Accordingly, there is also provided a method for modulating gene expression using the method according to the first aspect of the invention.

A further aspect of the invention provides a screening method to identify a drug target, comprising the steps of: providing a test drug target comprising a bromodomain; performing the method steps of the first aspect of the invention; determining whether the physiological function of the bromodomain of the test drug target has been selectively inhibited.

### DETAILED DESCRIPTION

The present invention will now be described with reference to the following non-limiting examples and figures, which show:

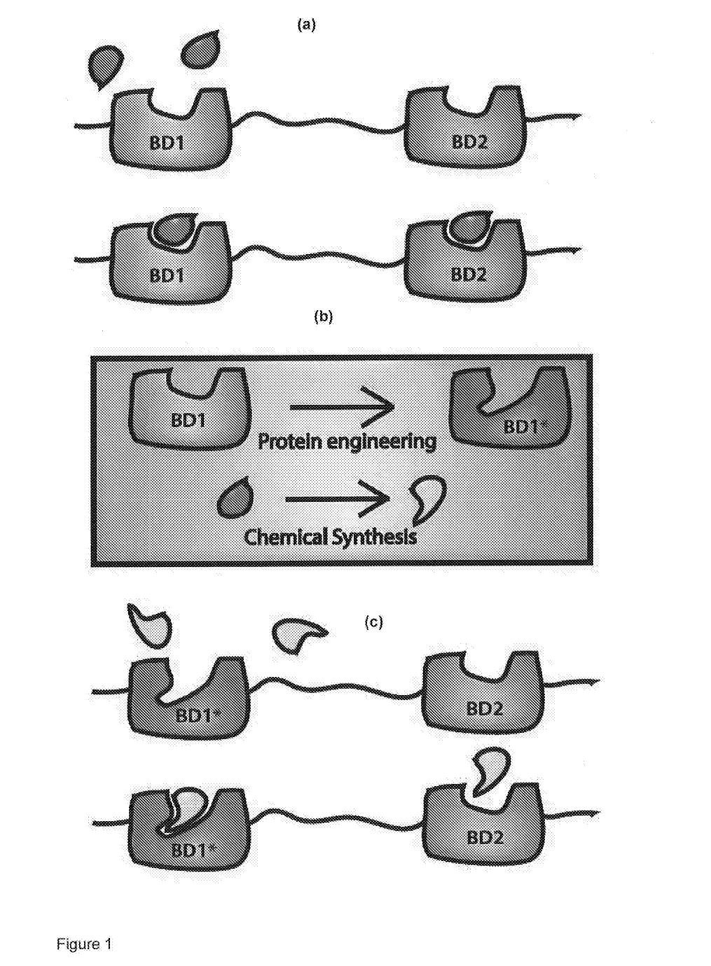

FIG. 1: Schematic illustration of the bump and hole approach. (a) shows BET-subfamily selective chemical probes bind with similarly high affinity towards all BET bromodomains, (b) shows introduction of ‘holes’ in the protein binding site via site directed mutagenesis, while simultaneously adding ‘bumps’ to existing ligands via chemical synthesis, (c) shows engineered specificity will allow modulation of individual BET bromodomains.

FIG. 2: Sequence alignment of eight BET bromodomains. Conserved residues and a conserved asparagine (position 140) that directly hydrogen bonds to acetyl-lysine, are highlighted. Conserved and non-conserved residues making contacts with iBET within the bromodomain binding site are highlighted with single black dots and asterisks, respectively.

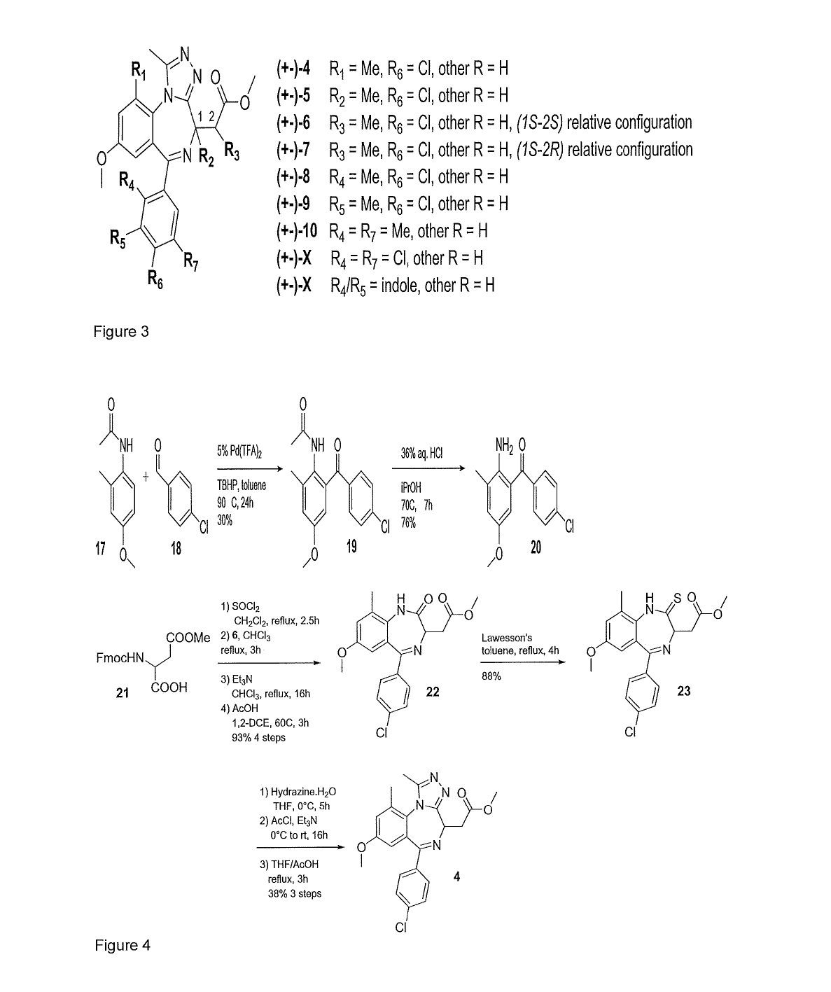

FIG. 3: Methyl scan showing derivatives synthesised.

FIG. 4: Synthesis of compound 4.

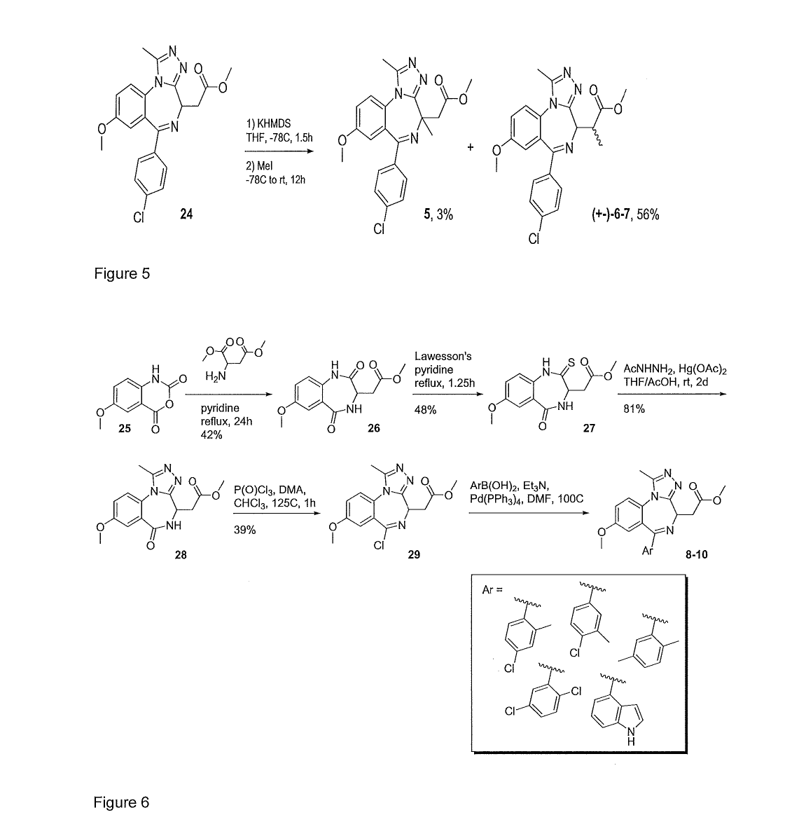

FIG. 5: Synthesis of compounds 5-7.

FIG. 6: Synthesis of compounds 8-10.

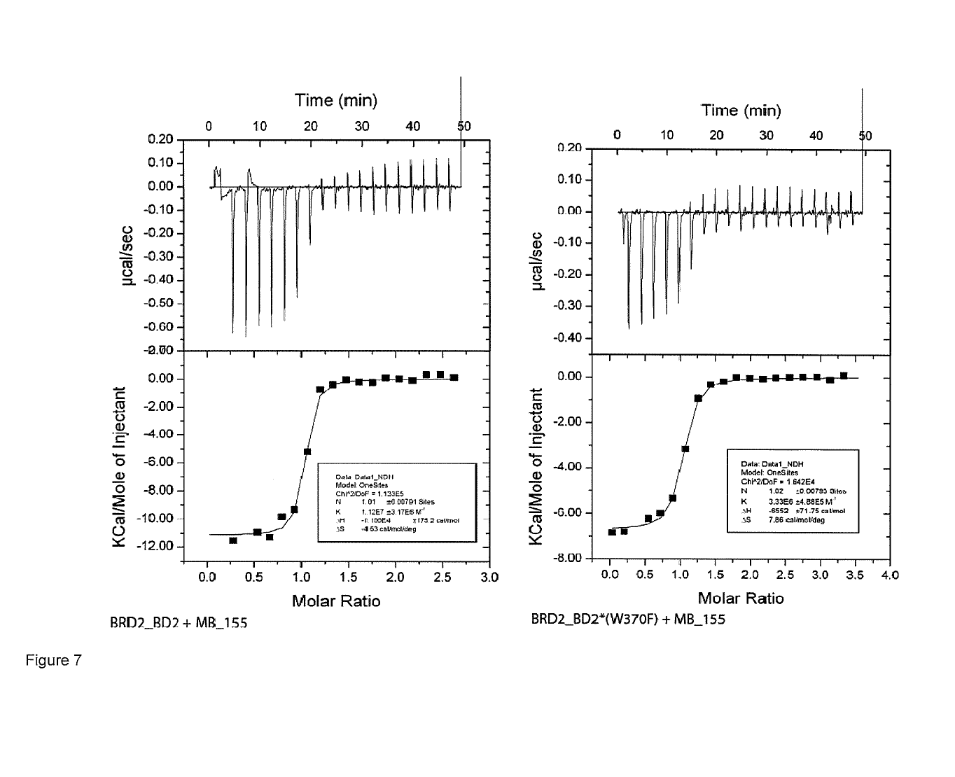

FIG. 7: ITC results—Brd2(2) and Brd2(2)*(Trp370Phe) (at 200 μM) into compound 18 (at 20 μM) at 25° C.

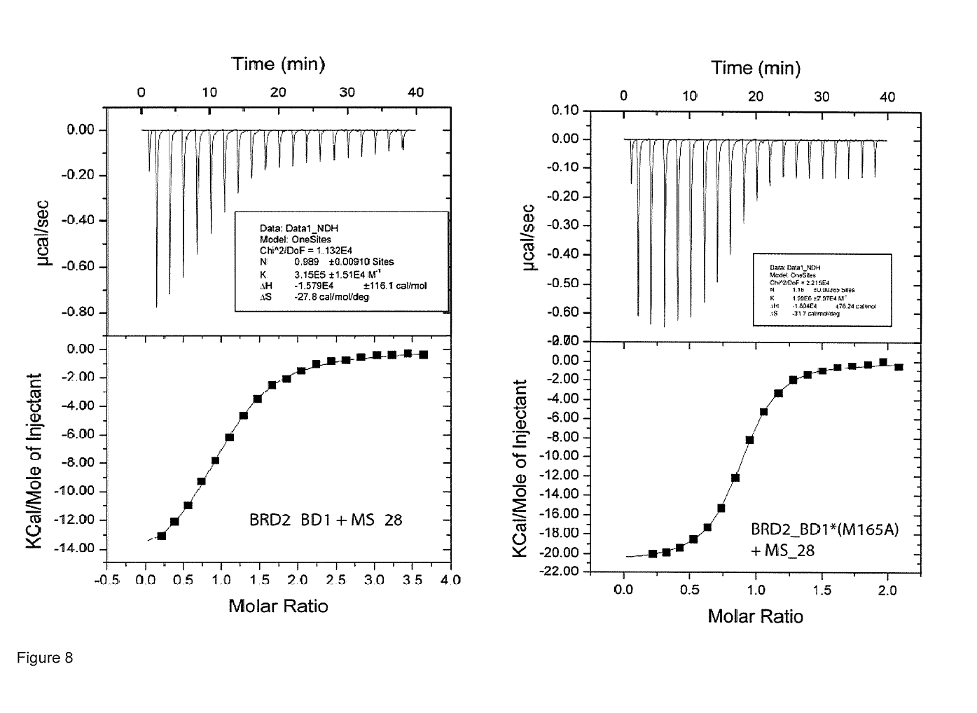

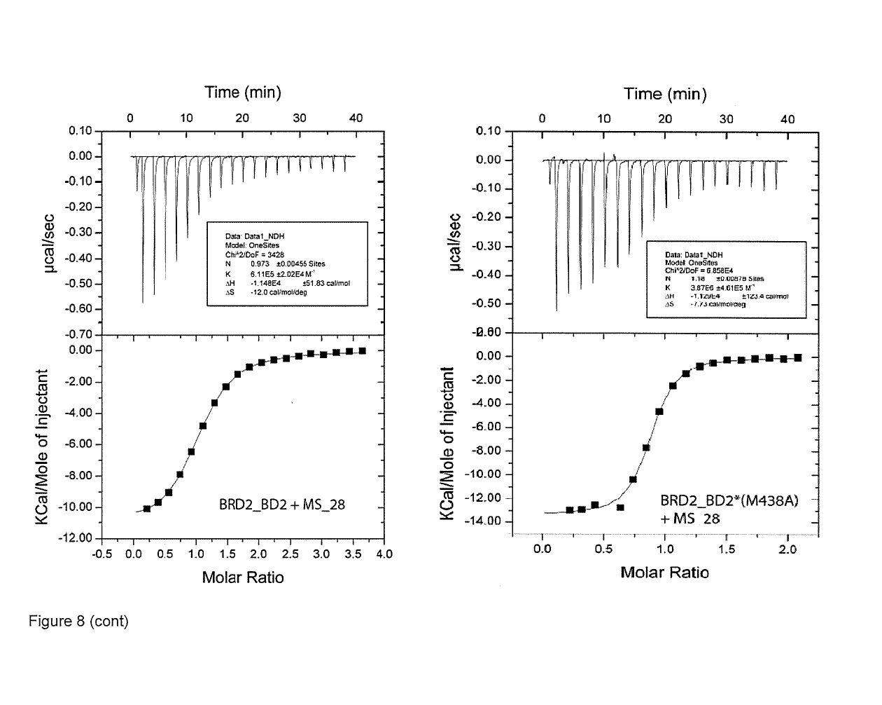

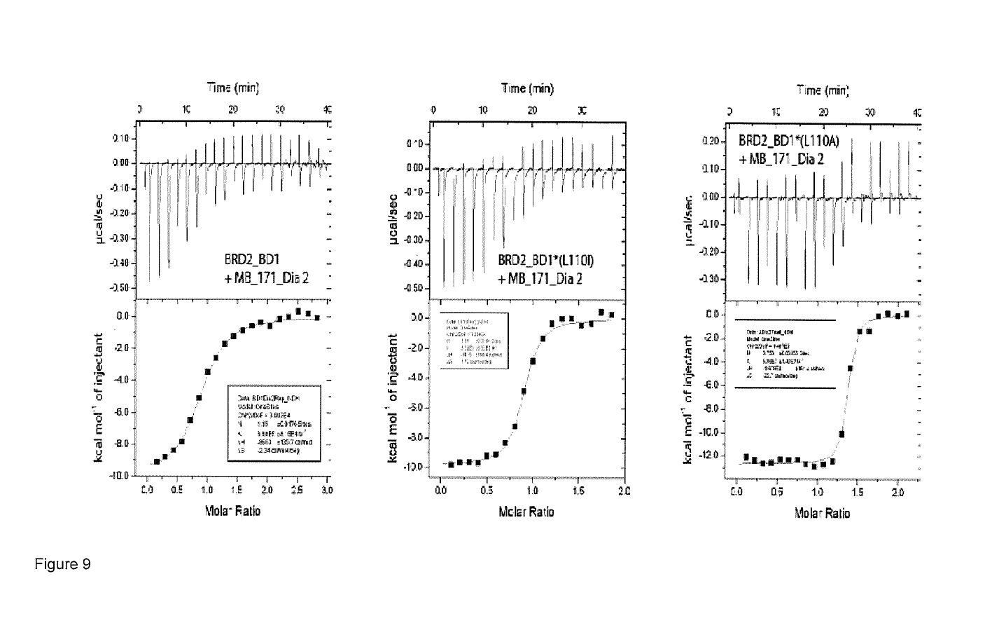

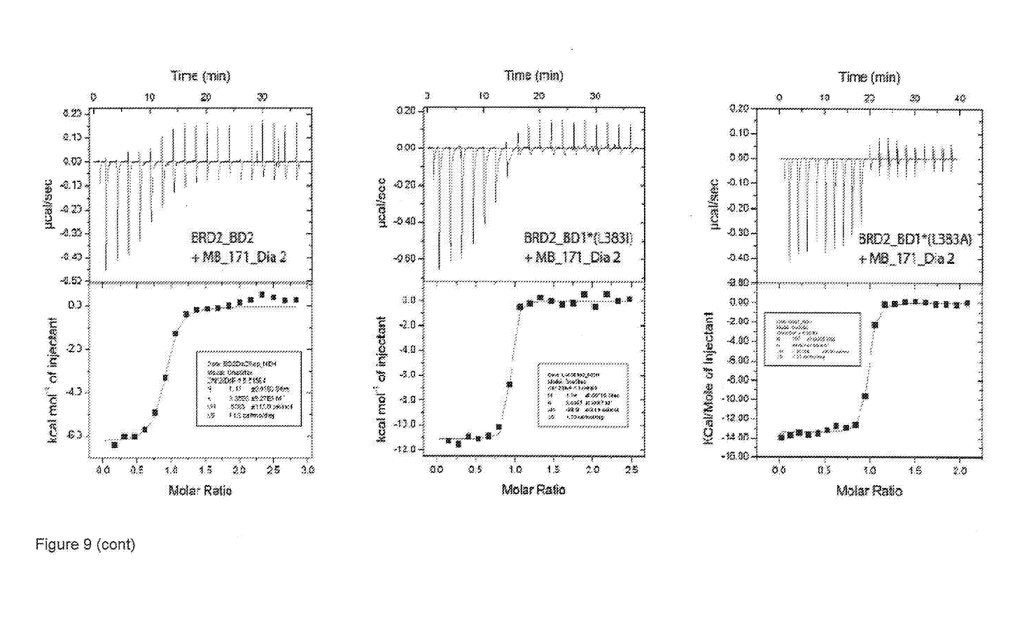

FIG. 8: ITC results—Titrations of Brd2 wild types and methionine mutants into compound 51 at 25° C.

FIG. 9: ITC results—Titrations of leucine mutants at 200 μM into a solution of 20 μM compound 7 at 25° C. Titrations of wild types at 350 μM into a solution of 20 μM compound 7 at 25° C.

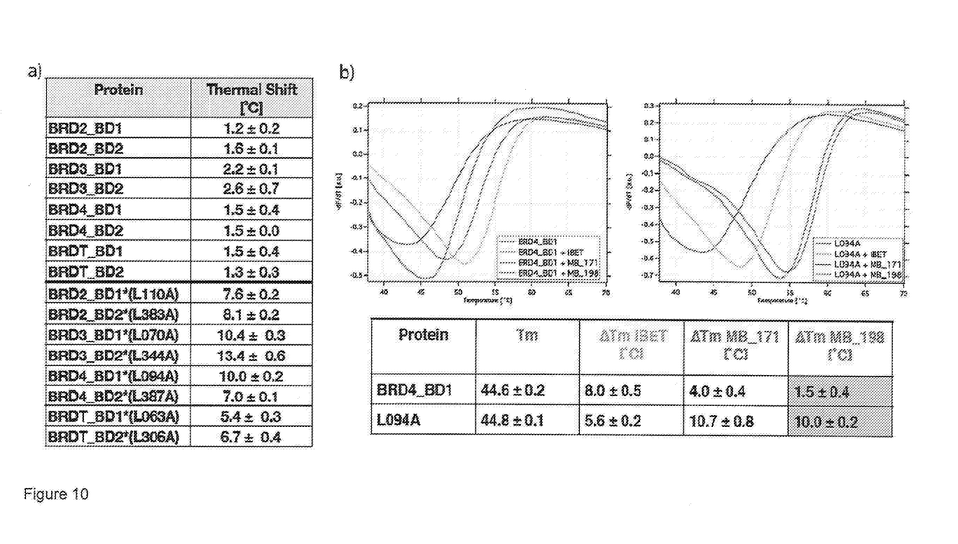

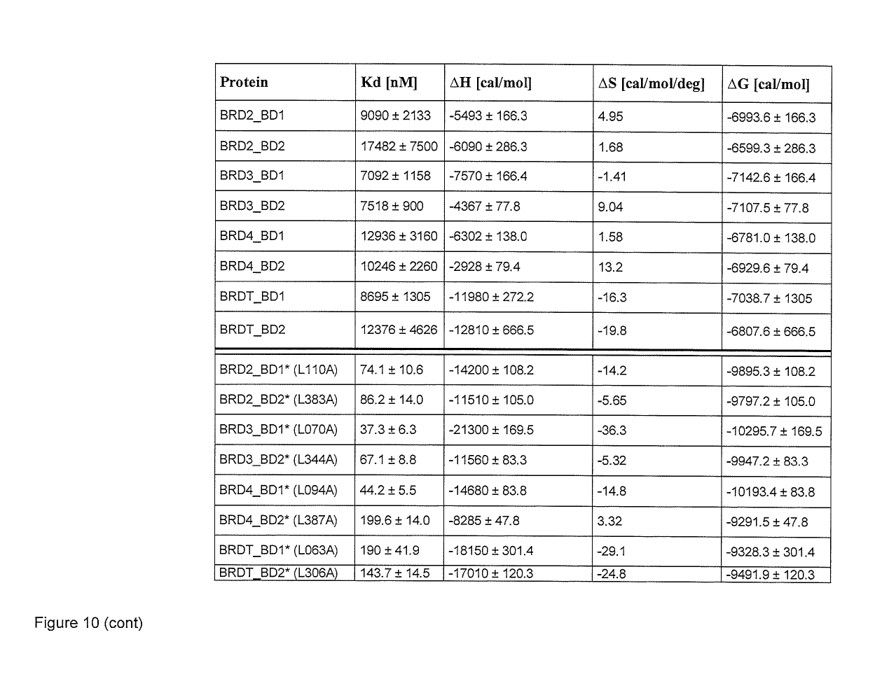

FIG. 10: DSF and ITC data obtained for all wild types and all leucine to alanine mutants with compound 11. ITC titrations data at 30° C. and 1% DMSO.

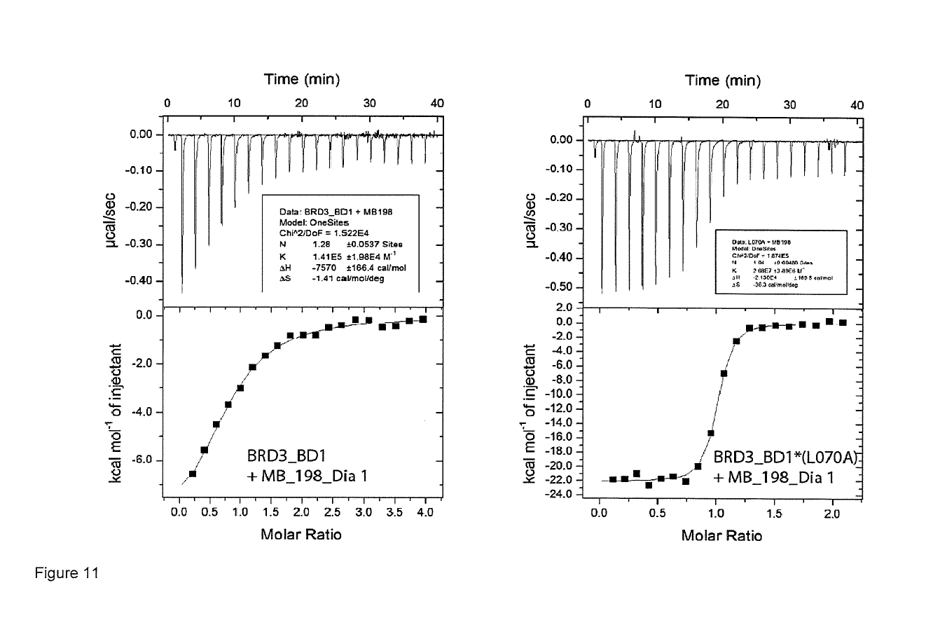

FIG. 11: ITC curves obtained for titrations of Brd3(1) and its respective leucine to alanine mutation into compound 11.

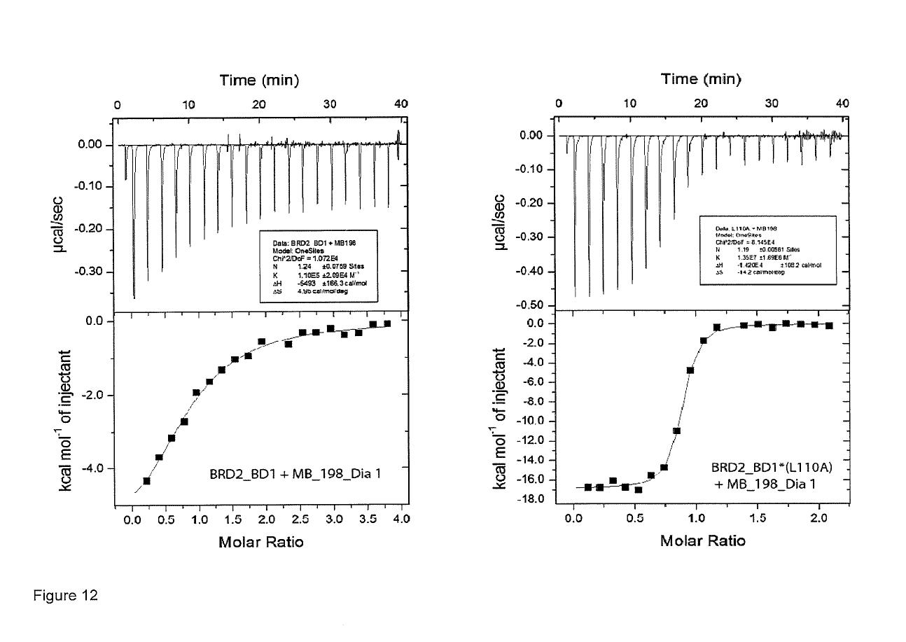

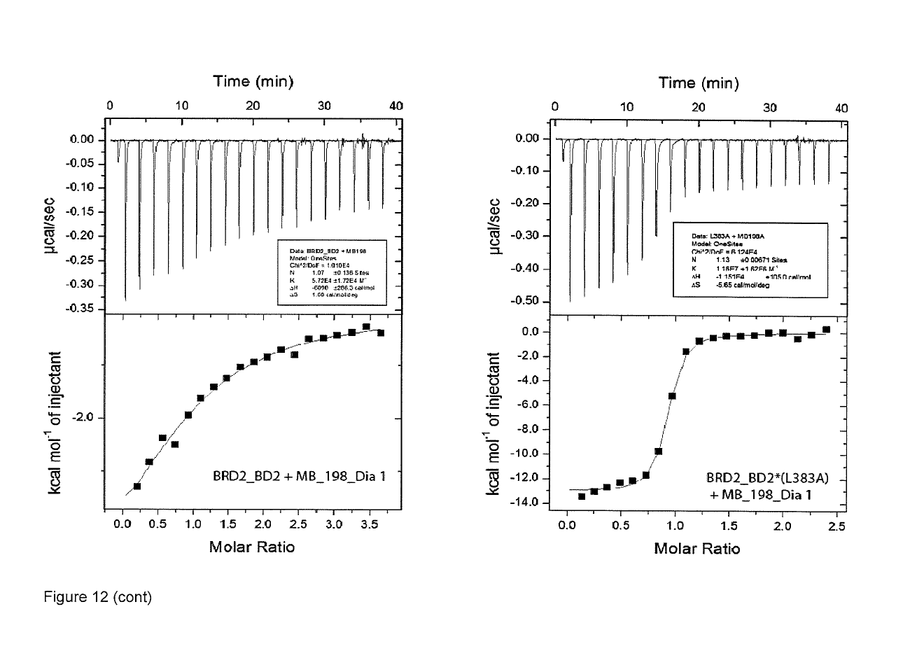

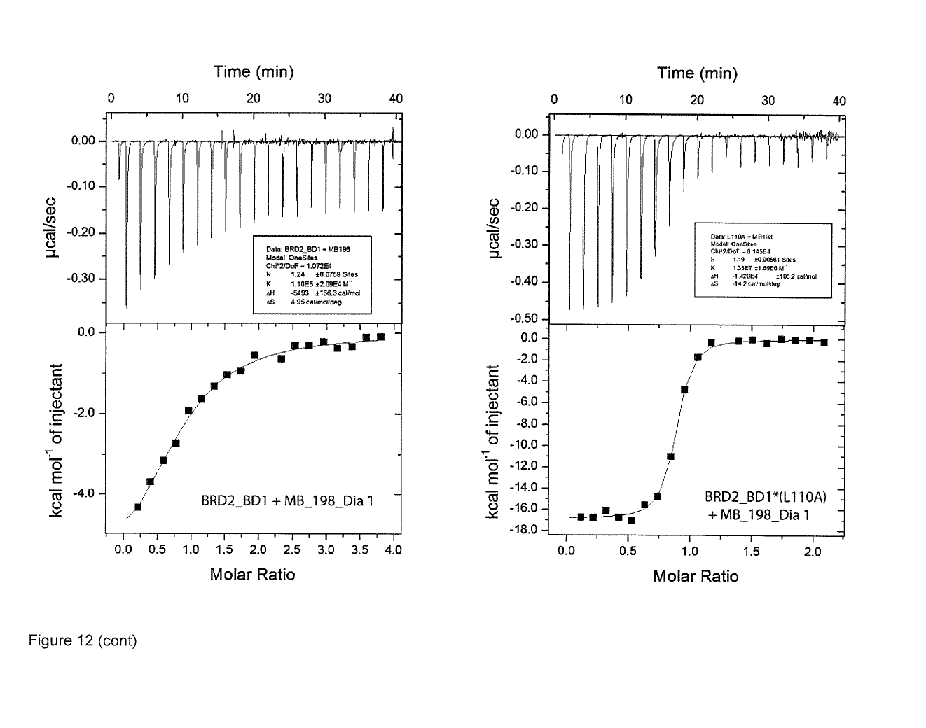

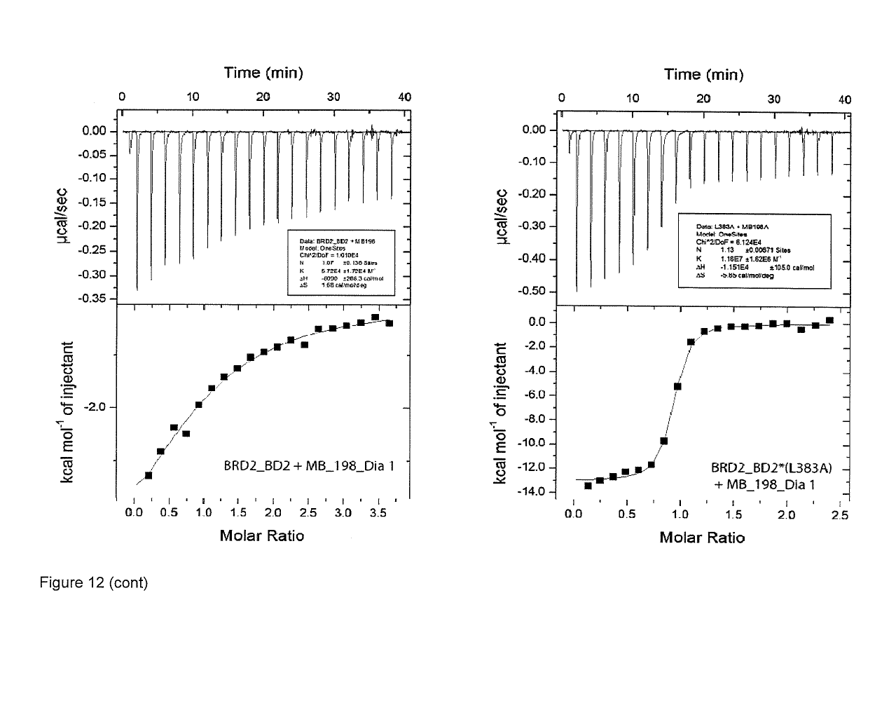

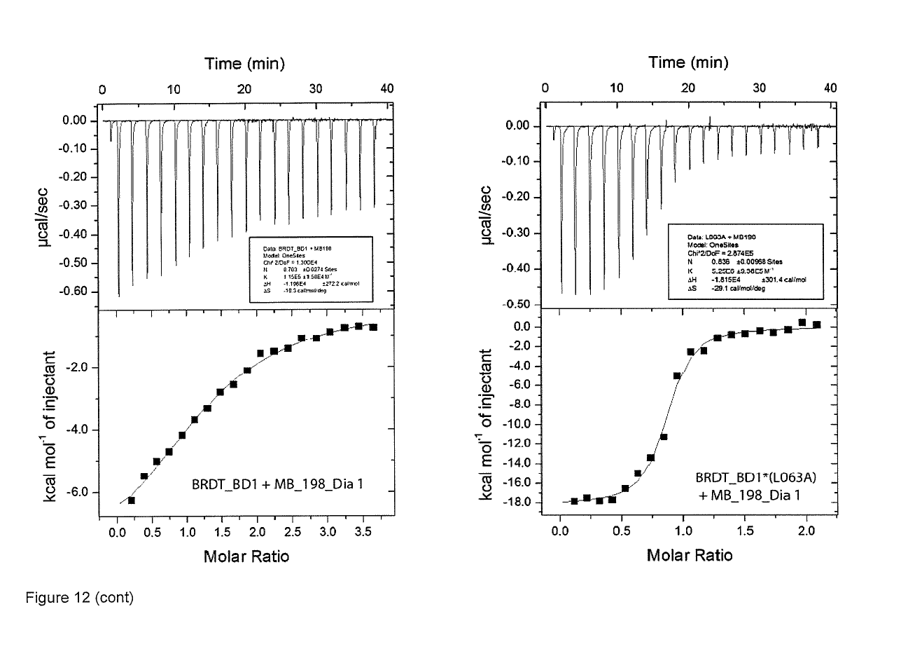

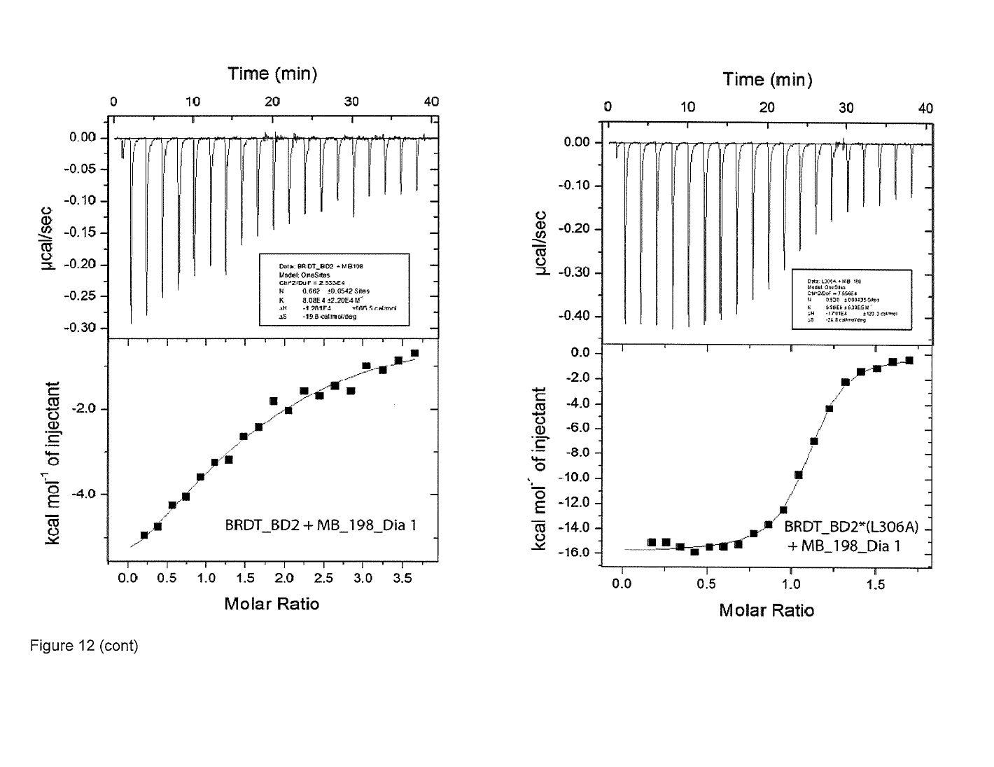

FIG. 12: ITC curves obtained for titrations of Brd2(1), Brd2(2), Brd4(1), Brd4(2), Brdt(1) and Brdt(2) and their respective leucine to alanine mutations into compound 11.

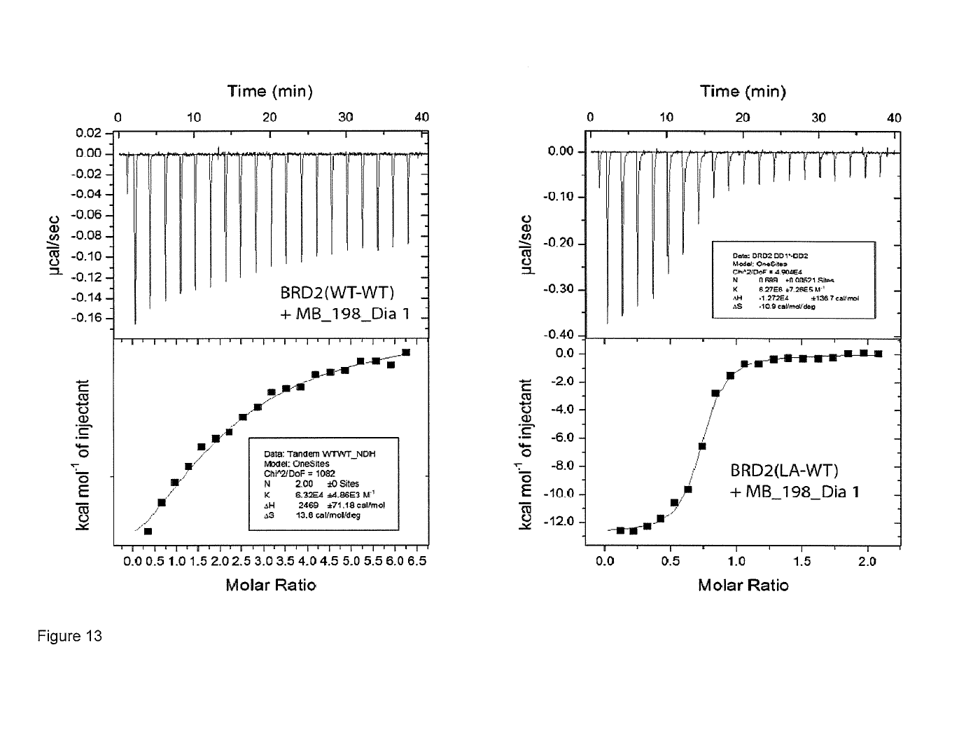

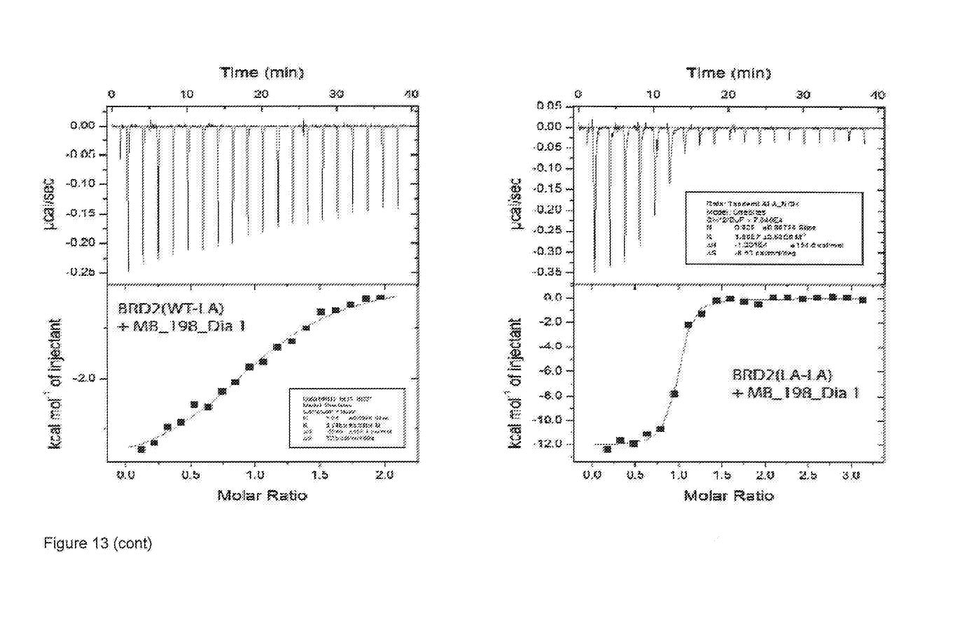

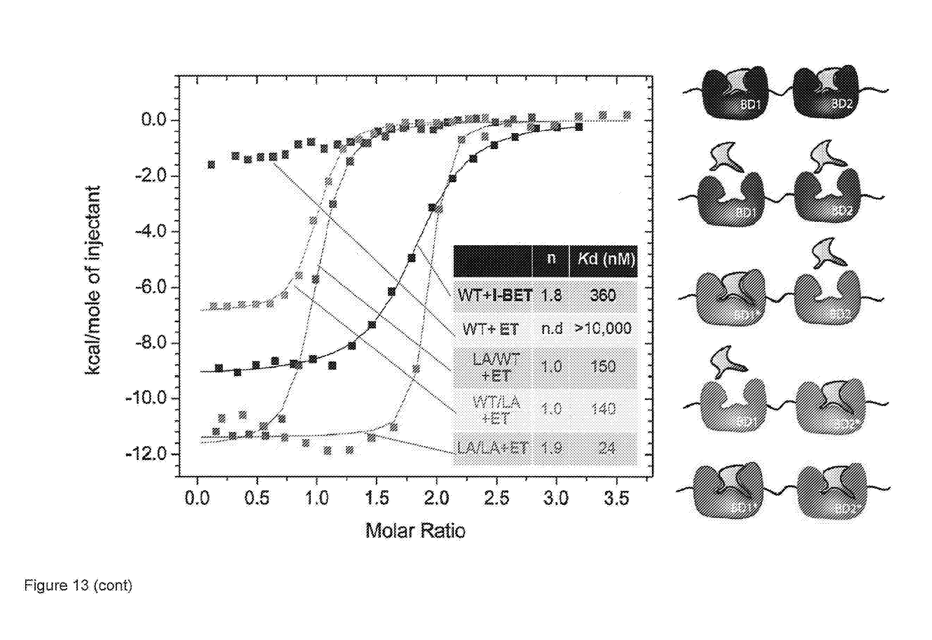

FIG. 13: ITC results for titrations of compound 11 (ET) into tandem constructs of BRD2 at 30° C. Shown in black is a control experiment of I-BET into wild type Brd2 tandem.

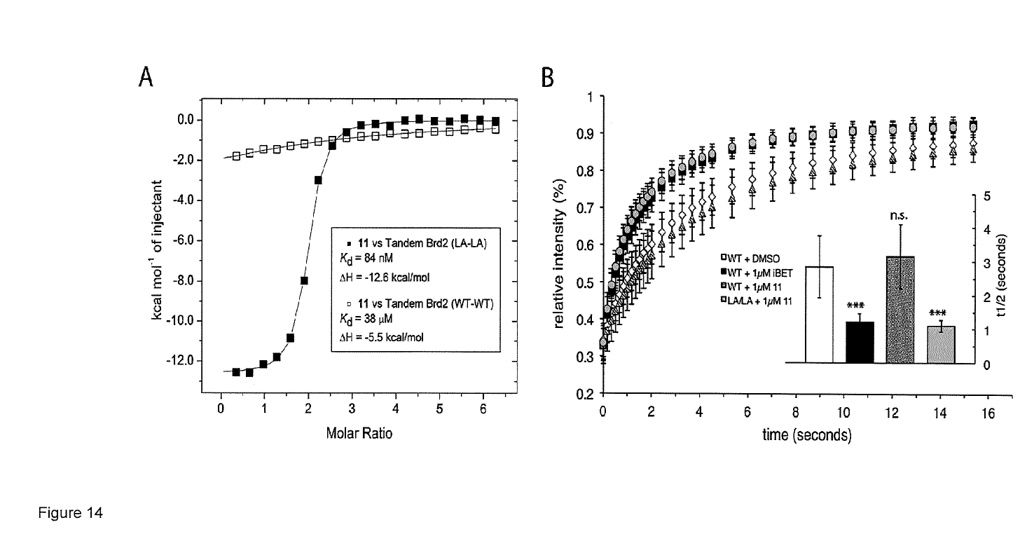

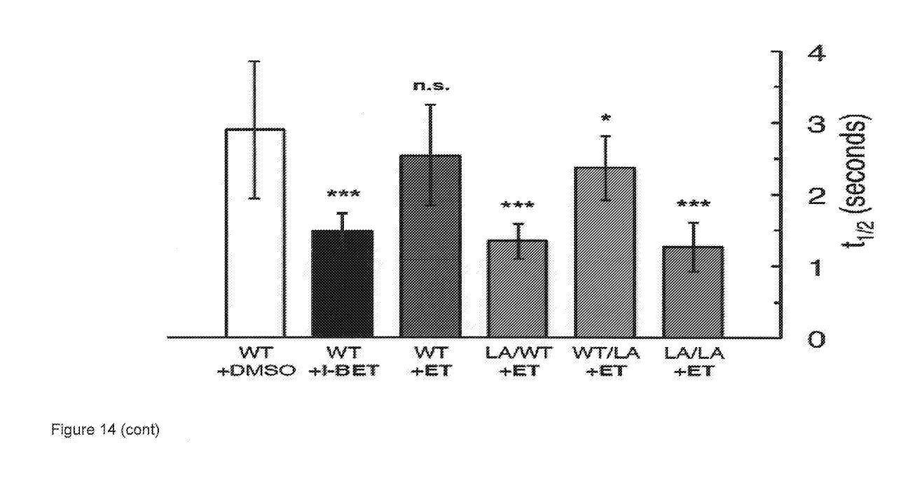

FIG. 14: Compound 11 (ET) is highly selective for a Leu/Ala mutant relative to WT BET bromodomains in vitro and in cells using FRAP. FRAP data demonstrates that selective blockade of the first bromodomain alone (but not of the second) is sufficient to displace Brd4 protein from chromatin.

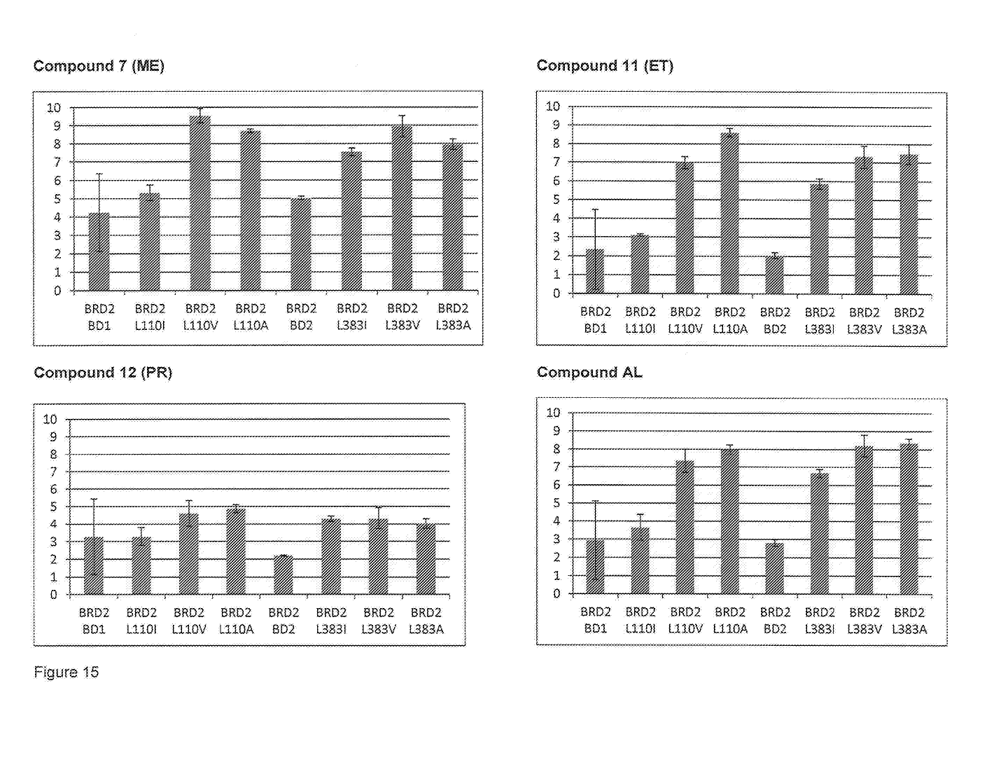

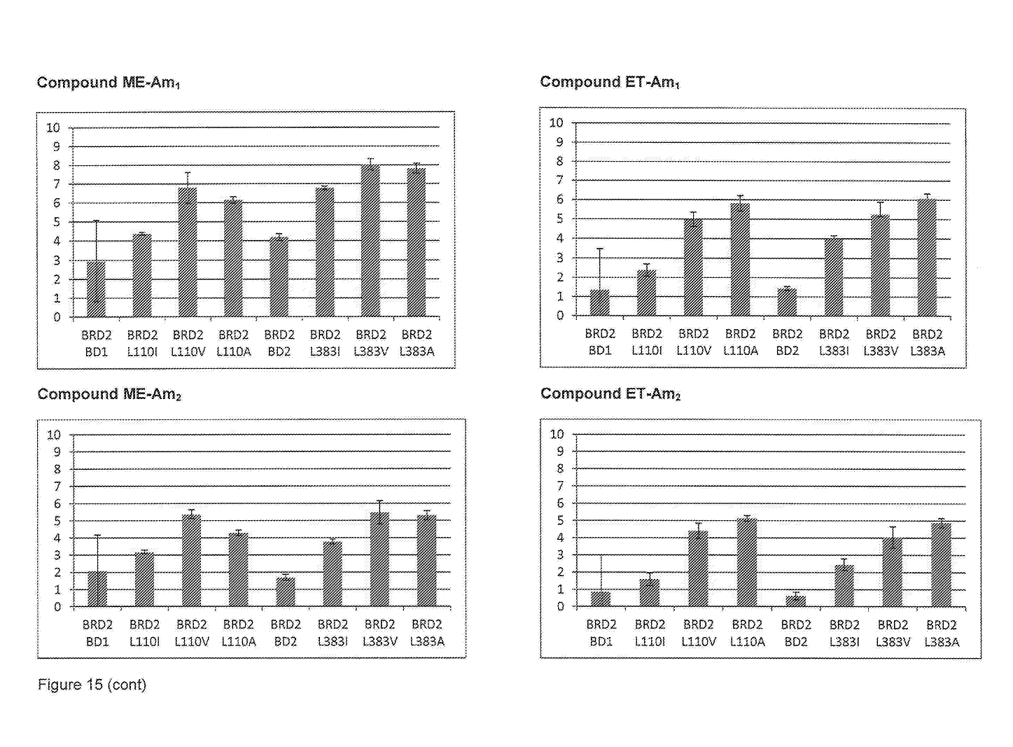

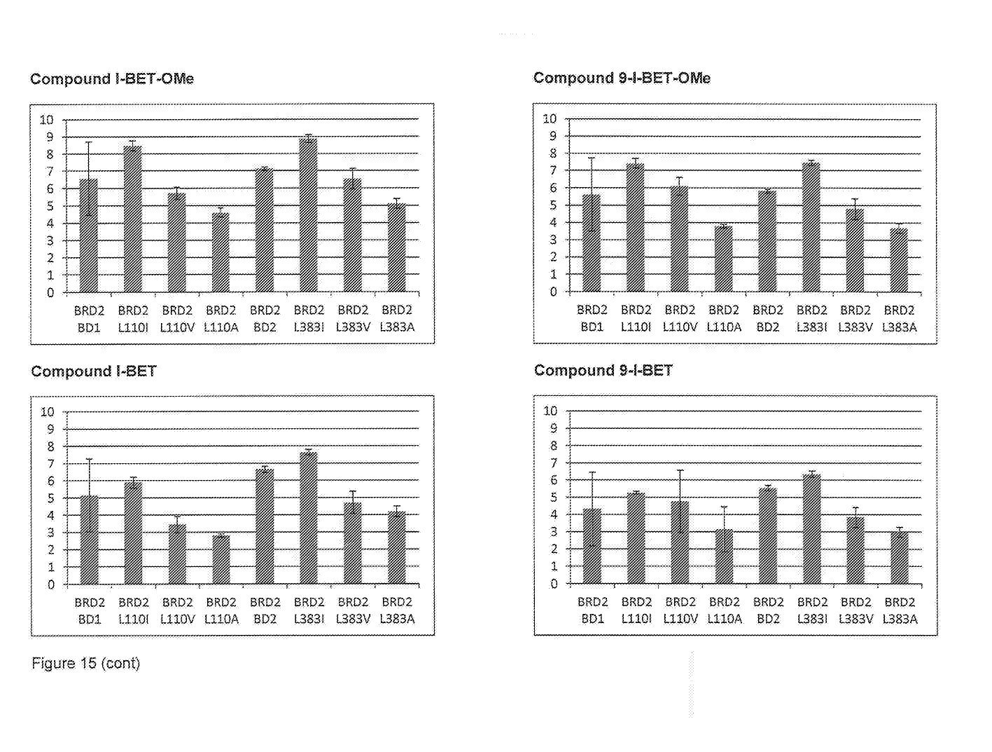

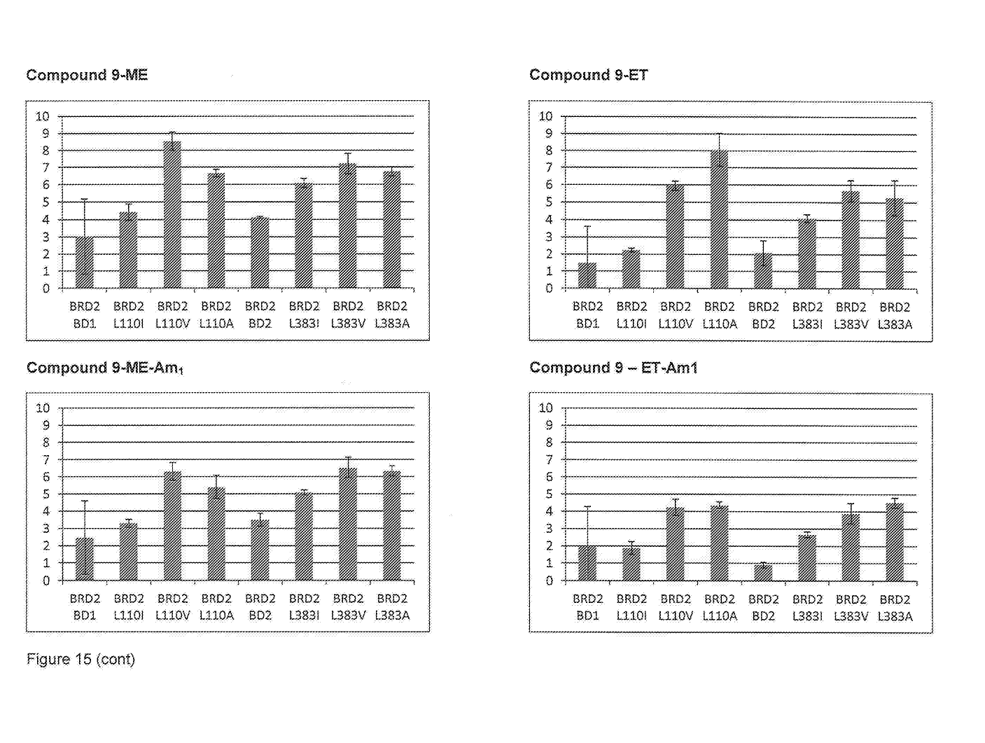

FIG. 15: Thermal shift data for Brd2 wild type and mutants in the presence of inhibitor candidates.

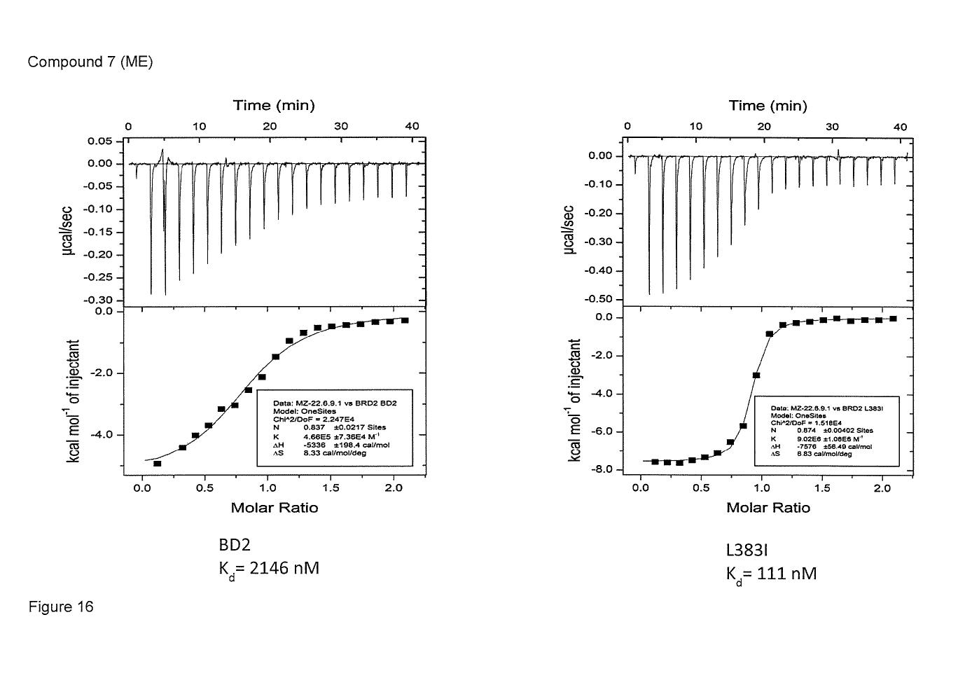

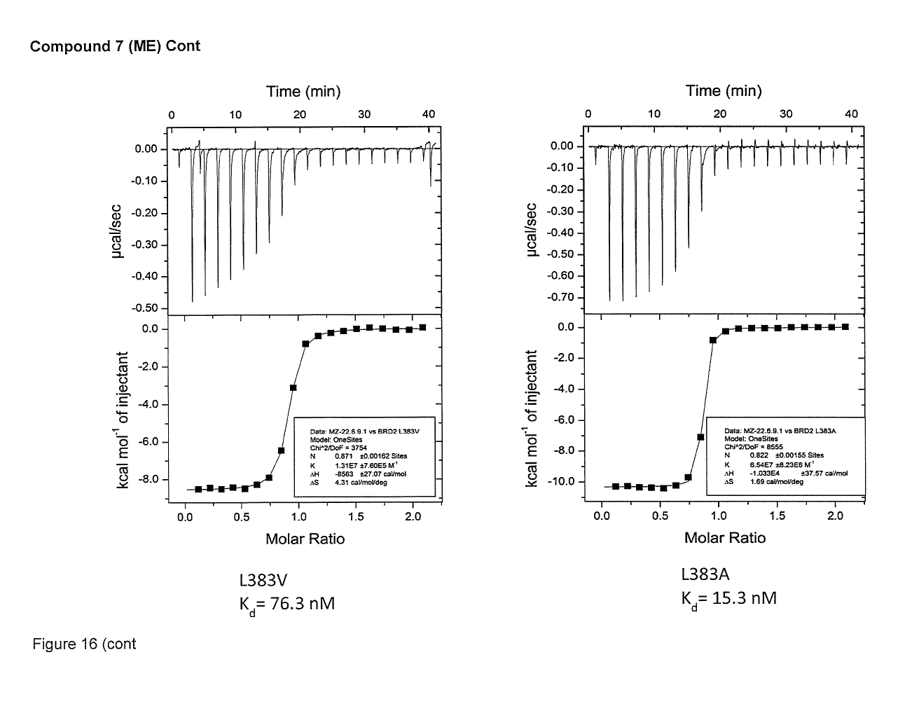

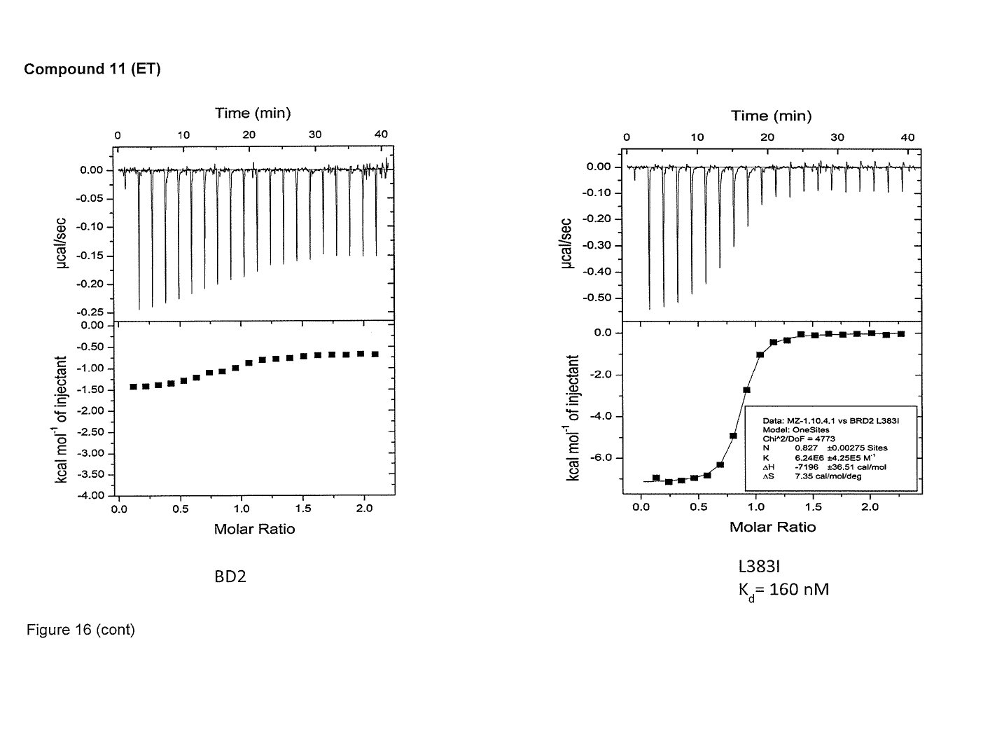

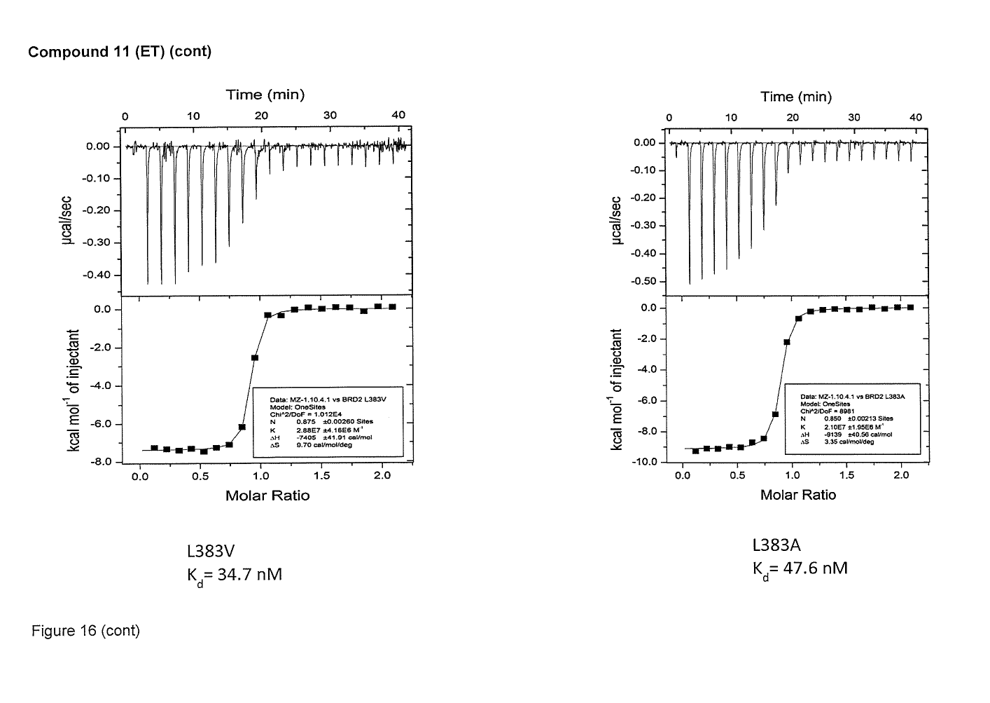

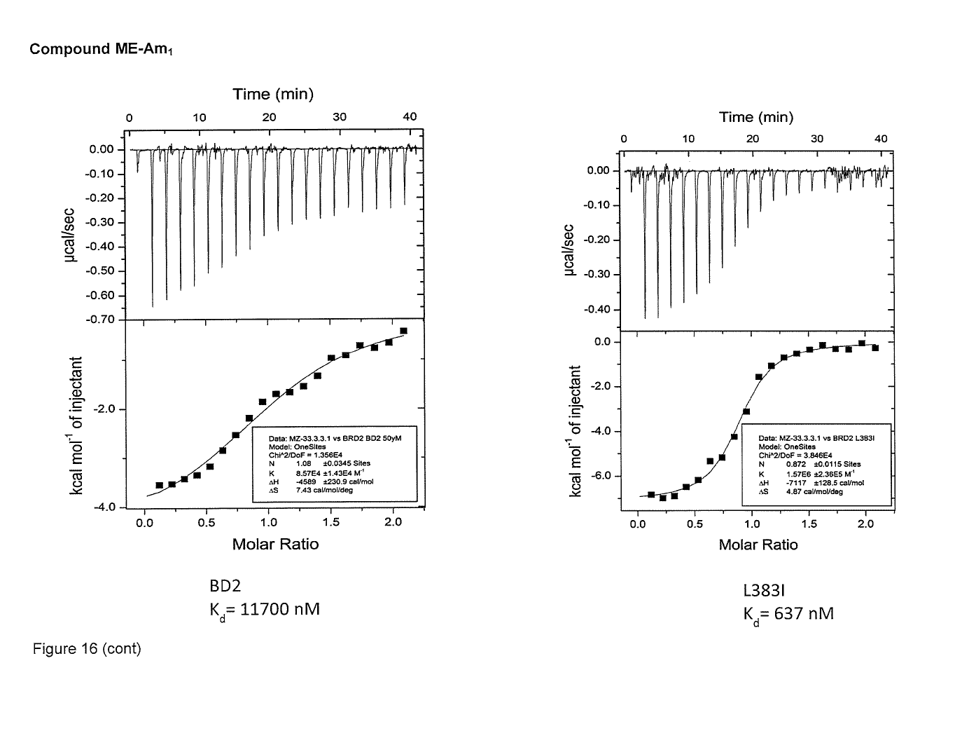

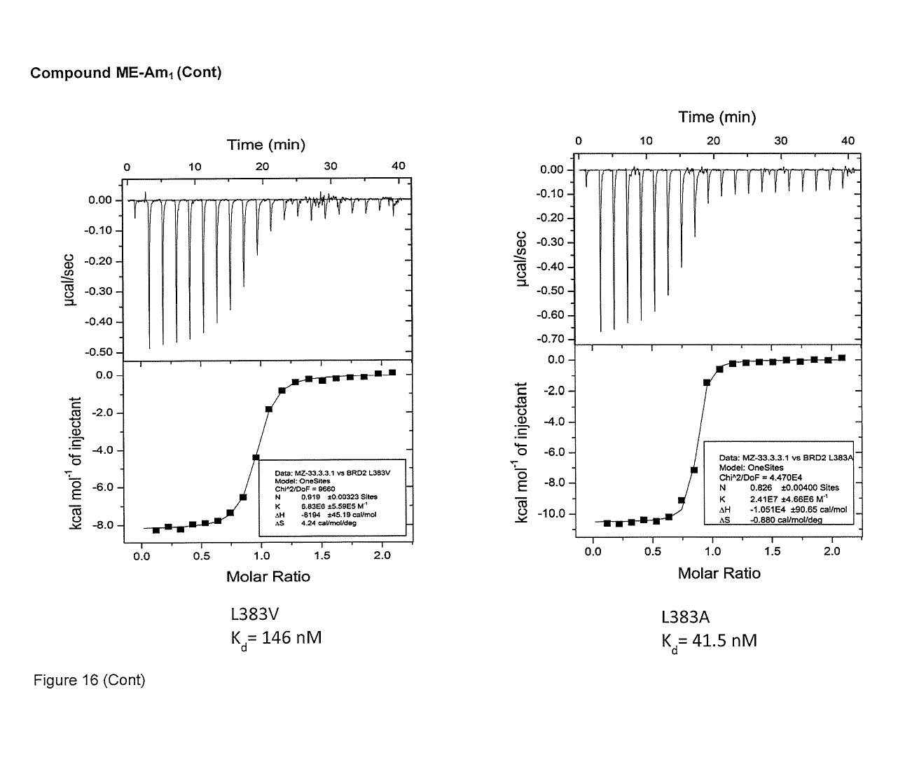

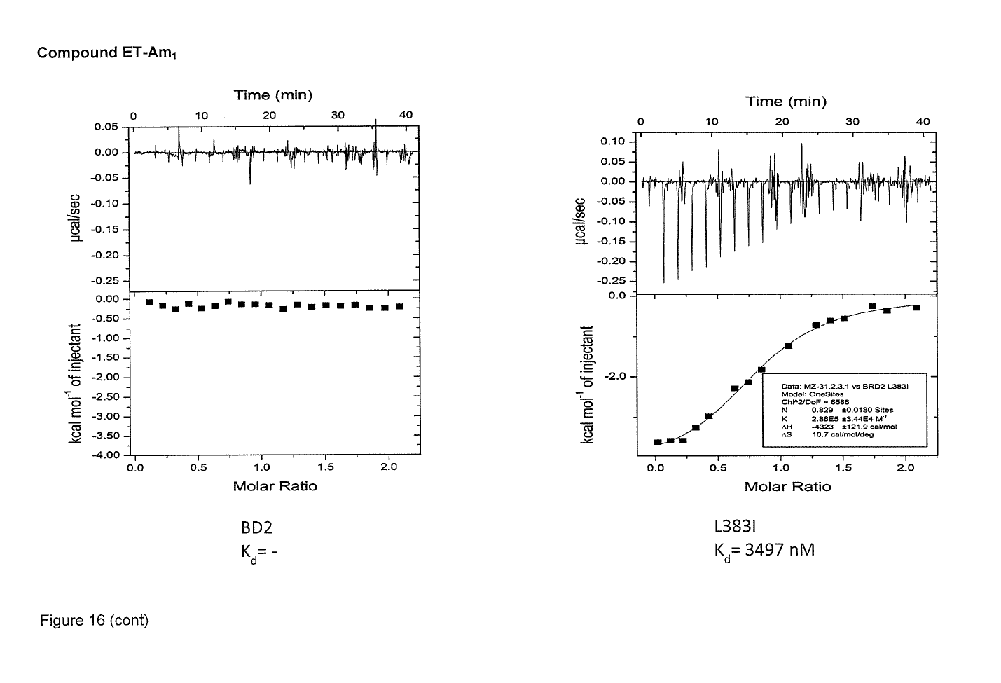

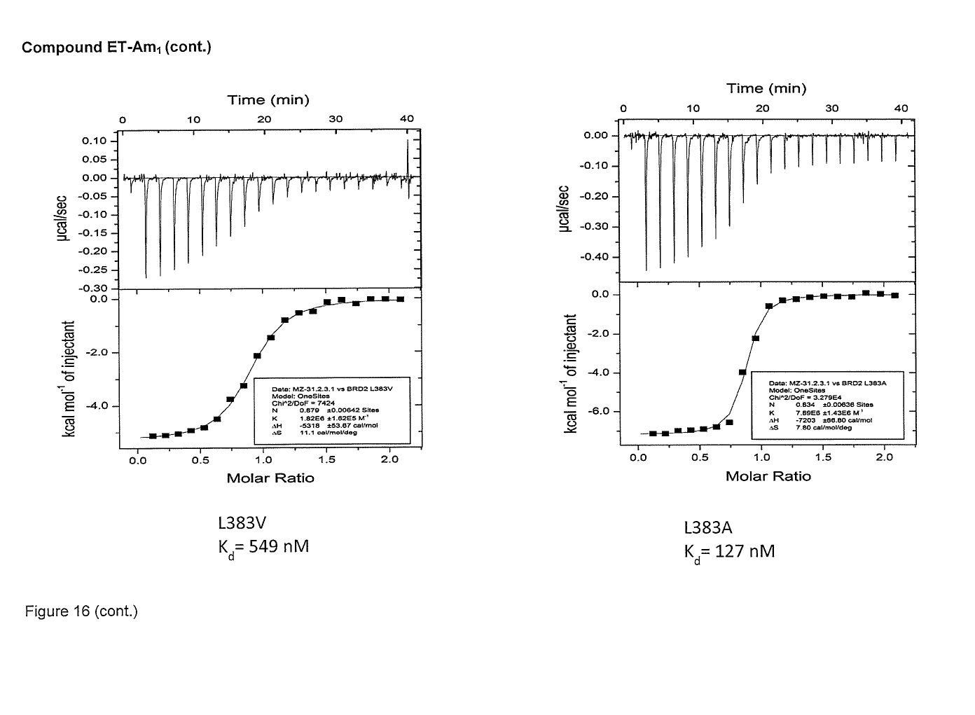

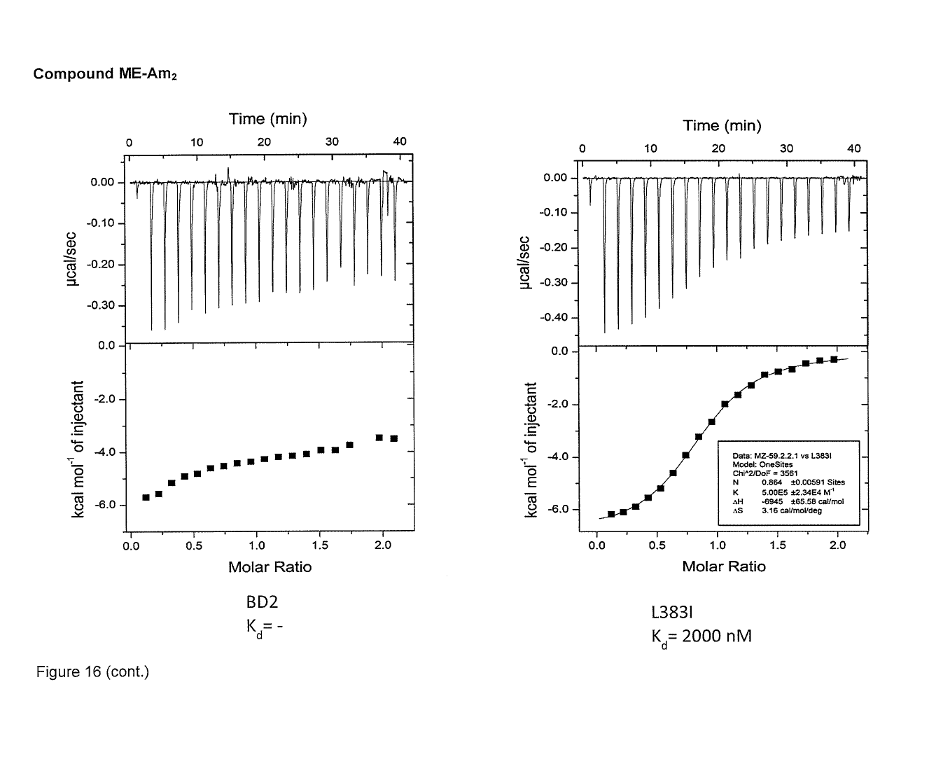

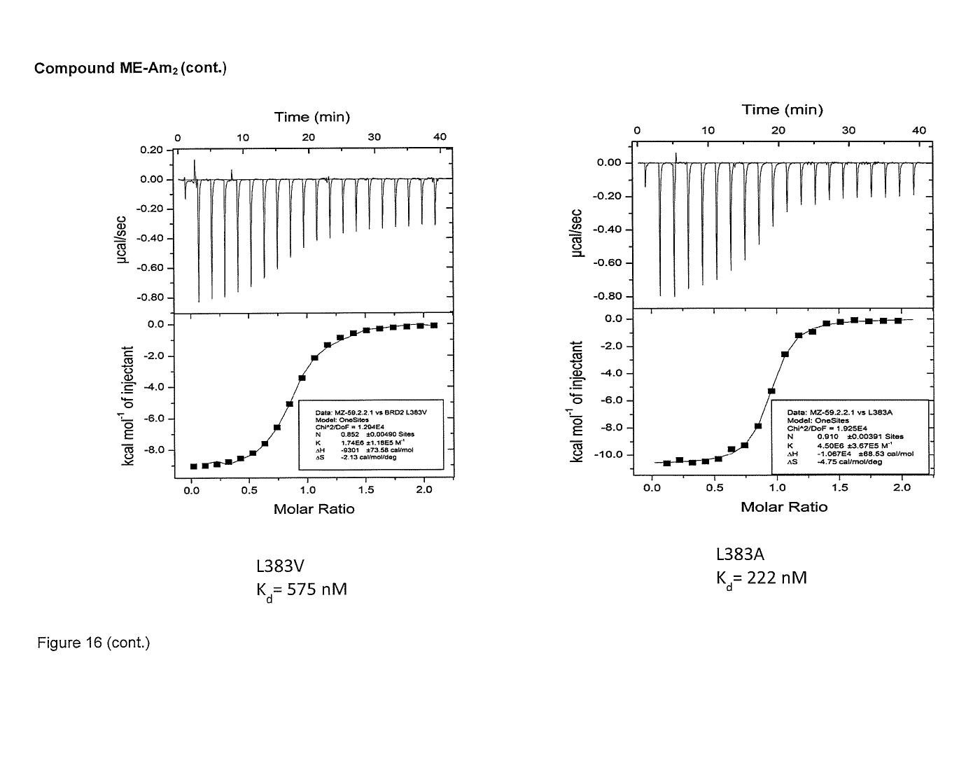

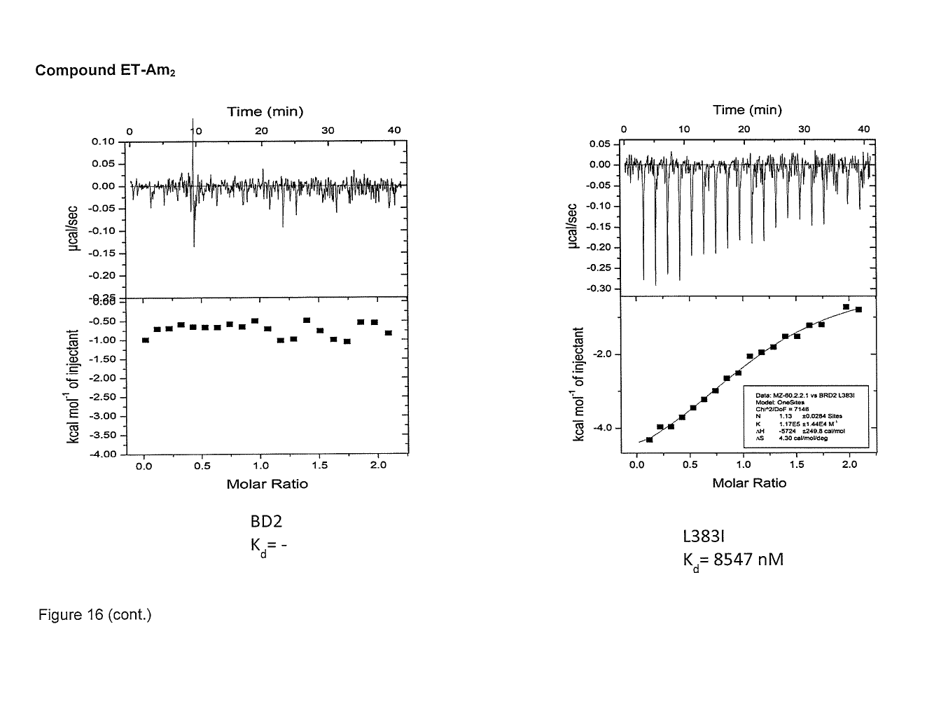

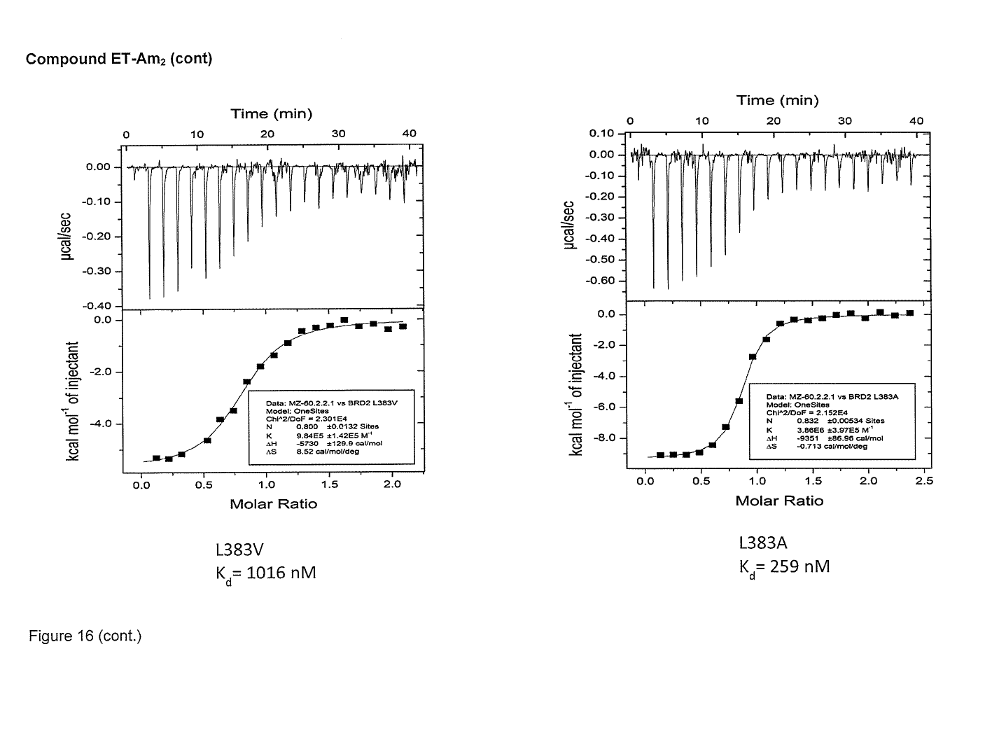

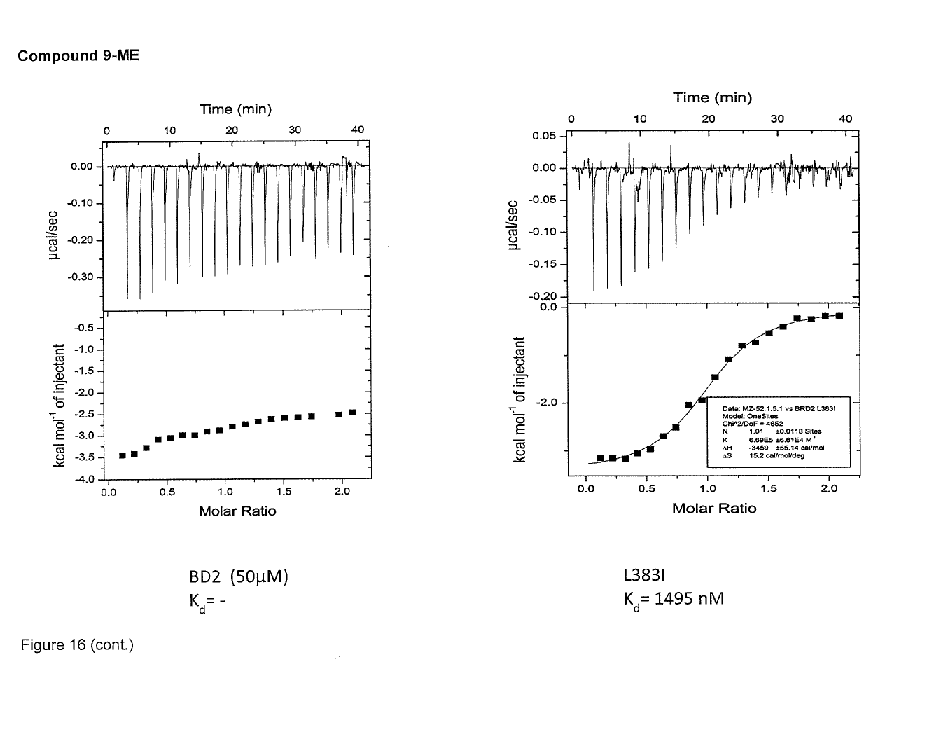

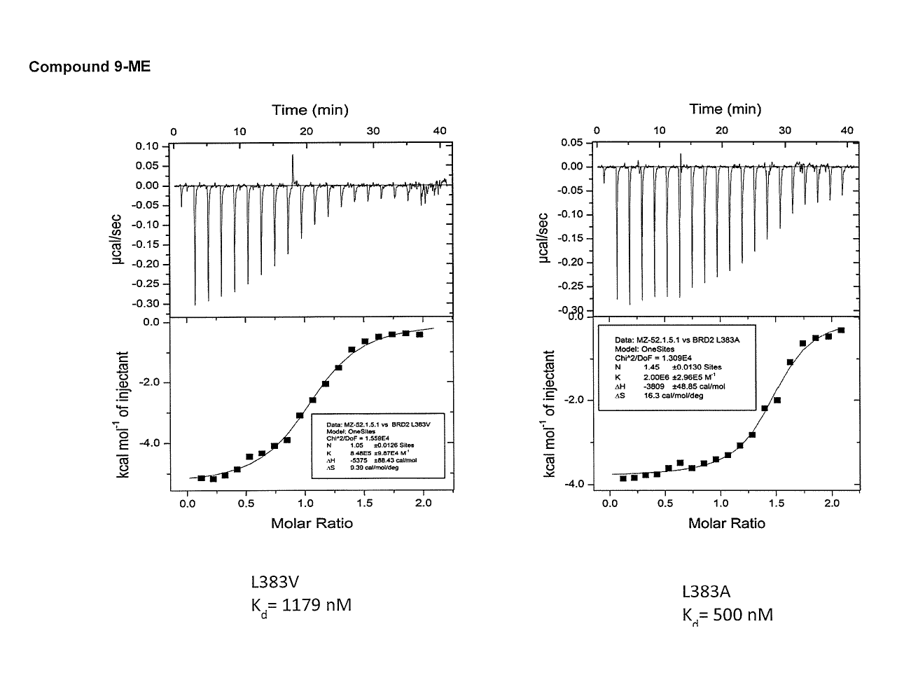

FIG. 16: ITC data for Brd2(2) wild type and mutants in the presence of inhibitor candidates.

### ABBREVIATIONS

### BET—Bromo and Extra-Terminal

### DSF—Differential Scanning Fluorimetry

ITC—Isothermal Titration calorimetry

### SENP1—Sentrin-specific Protease 1

### SGC—Structural Genomics Consortium

### SUMO—Small Ubiquitin-like Modifier

### TEV—Tobacco Etch Virus

### Results

The present inventors have conducted experiments to investigate how the physiological role of a single bromodomain within a protein can be elucidated. If this can be achieved, such domains could potentially be confirmed as targets for drug discovery.

The inventors have devised a “bump and hole” approach (FIG. 1), wherein a phenotypically silent mutation is introduced into the bromodomain of interest. The mutation introduces a side pocket within the bromodomain binding site, which otherwise retains wild type functionality. An inhibitor which is complementary to the altered binding site can then be developed and used to selectively inhibit the mutant domain, whilst not binding (or binding less strongly) to the wild type domain as a result of steric clash with the naturally occurring residue.

### Hole Design

In order to investigate how a bromodomain might be specifically targeted for inhibition, the present inventors inspected the primary amino acid sequences of the eight BET bromodomains (FIG. 2) as well as the crystal structures of iBET and iBET and JQ1 bound to BET bromodomains (not shown). Analyses of these sequences and structural alignments highlighted the presence of several conserved residues within the BET subfamily that are related in sequence and space, and a conservation of ligand binding modes around the common triazolodiazepine scaffold, suggesting that a “bump and hole” approach might be feasible. The present inventors focussed initially on eleven strictly conserved residues that would be in close contact with iBET/JQ1, keeping in mind that the introduced mutations should not significantly disrupt protein stability and wild type histone binding.

Residues tyrosine 97, cysteine 136, tyrosine 139 and asparagine 140 (Brd4(1) numbering used throughout unless otherwise specified) were readily discarded, as these positions are known to be important for KAc recognition [6] and for preserving a key network of bound water molecules deep in the KAc binding pocket [7]. Buried proline 82 and phenylalanine 83 from the bottom of the so-called WPF shelf were also discarded as their mutation was predicted by us and others [8] to destabilize the integrity of the hydrophobic core. This analysis left the more peripheral, hydrophobic residues Tryptophan 81 from the top of the WPF shelf, and Valine 87 and Leucine 94 from the ZA loop, to be selected as candidates for mutagenesis.

Mutants Tryptophan/Phenylalanine, Tryptophan/Histidine, Valine/Alanine, Valine/Glycine, Leucine/Isoleucine, Leucine/Alanine and Leucine/Glycine were initially constructed within Brd2(1) and Brd2(2) as model systems (Table A), expressed and purified from E. coli and biophysically characterized in order to assess their functionality both in terms of stability and histone peptide binding. Methionine 149 was also investigated, with Methionine/Alanine and Methionine/Leucine mutants being constructed into Brd2(1) and Brd2(2) for testing (Table A).

TABLE A
Site directed mutagenesis of bromodomains BRD2_BD1,
BRD2_BD2, BRD4_BD1 and BRD4_BD2.

| BRD2_BD1 | BRD2_BD2 | BRD4_BD1 | BRD4_BD2 |
|---|---|---|---|
| Trp097-Phe | Trp-370-Phe | Trp081-Phe | Trp374-Phe |
| Trp097-His | Trp370-His | Trp081-His | Trp374-His |
| Val103-Ala | Val376-Ala | Val087-Ala | Val380-Ala |
| / | Val376-Gly | / | / |
| Leu110-Ala | Leu383-Ala | Leu094-Ala | Leu387-Ala |
| Leu110-Ile | Leu383-Ile | Leu094-Ile | Leu387-Ile |
| Leu110-Gly | / | / | / |
| Met165-Ala | Met438-Ala | / | / |
| Met165-Leu | Met438-Leu | / | / |

Protein stability was confirmed by differential scanning fluorimetry (DSF). Pleasingly, the mutant proteins were all found to be stable at temperatures greater than 40° C., albeit with some loss in stability relative to the wild type protein (Table B). The retention of wild type functionality by the mutant proteins was tested by Isothermal Titration calorimetry (ITC) titrations of 1-2 mM tetra acetylated peptide into 50-100 μM protein at 15° C. (results shown Table B).

TABLE B
Biophysical characterization of Brd2-BD1 and Brd2-BD2 mutants and their
binding to histone peptides. Melting temperature (Tm), variation of Tm compared to the
respective wild type (ΔTm) and thermodynamic parameters for the binding of the different
proteins to a tetra-acetylated H4 derived peptide (18) are given. Conditions: TS) 2 μM of WTs
and mutants were submitted to a temperature ramp from 37° C. to 95° C. in the presence or
absence of 100 μM peptide. ITC) titration of peptide (1-2 mM) into WT and mutants (50-100 μM)
at 15° C. Conditions: TS) 2 μM of WTs and mutants were submitted to a temperature ramp from
37° C. to 95° C. in the presence or absence of 100 μM peptide. ITC) titration of peptide (1-2 mM)
into WT and mutants (50-100 μM) at 15° C.

| | | ΔTₘ from | H₂N−YSGRGK(Ac)GGK(Ac)GLGK(Ac)GGAK(Ac)RHRK−COOH | | | |
|---|---|---|---|---|---|---|
| brd | Tₘ (° C.) | WT | K_{d} (μM) | ΔG (cal/mol) | ΔH (cal/mol) | ΔS (cal/mol/°) |
| brd2(1) | 46.2 ± 0.2 | / | 12.9 ± 2.30 | −6460 ± 90 | −11300 ± 500 | −16.9 ± 1.5 |
| brd2(1)_{V103A} | 41.1 ± 0.2 | −5.1 ± 0.4 | 286 ± 16 | −4680 ± 40 | −4800 ± 200 | −0.4 |
| brd2(1)_{L110I} | 43.8 ± 0.0 | −2.4 ± 0.2 | 16.3 ± 1.50 | −6310 ± 50 | −12500 ± 400 | −21.5 ± 1.2 |
| brd2(1)_{L110A} | 43.7 ± 0.0 | −2.5 ± 0.2 | 31.3 ± 17.3 | −5930 ± 600 | −2820 ± 615 | 10.8 ± 0.7 |
| brd2(1)_{W097F} | 44.7 ± 0.1 | −1.5 ± 0.3 | 25.9 ± 7.30 | −6050 ± 140 | −3880 ± 470 | 7.5 ± 1.1 |
| brd2(1)_{W097H} | 45.8 ± 0.1 | −0.4 ± 0.3 | 60.2 ± 3.80 | −5560 ± 35 | −5410 ± 140 | 0.5 ± 0.4 |
| brd2(1)_{M165A} | 42.8 ± 0.2 | −3.4 ± 0.2 | 15.9 ± 10.5 | −6340 ± 180 | −1350 ± 180 | 17.3 |
| brd2(1)_{M165L} | 42.4 ± 0.2 | −3.8 ± 0.2 | 55.9 ± 2.2 | −5620 ± 970 | −14200 ± 1000 | −29.7 |
| brd2(2) | 47.5 ± 0.1 | / | 150 ± 15.1 | −5040 ± 60 | −9200 ± 1700 | −14.5 ± 5.8 |
| brd2(2)_{V376A} | 43.5 ± 0.1 | −4.0 ± 0.2 | / | / | / | / |
| brd2(2)_{L383I} | 43.4 ± 0.1 | −4.1 ± 0.2 | / | / | / | / |
| brd2(2)_{L383A} | 44.8 ± 0.1 | −2.7 ± 0.2 | 89.3 ± 6.60 | −5330 ± 40 | −2240 ± 100 | 10.8 ± 0.2 |
| brd2(2)_{W370F} | 45.1 ± 0.0 | −2.4 ± 0.1 | / | / | / | / |
| brd2(2)_{W370H} | 45.5 ± 0.2 | −2.0 ± 0.3 | / | / | / | / |

Most mutants were observed to retain a similar affinity towards the histone peptide as the wild type construct, with 2 digit μM Kds by ITC. The leucine to isoleucine mutation at position 110 (Brd2(1)) showed the least change in thermodynamic parameters, suggesting that it is very stable and that this particular mutation does not alter binding to the peptide. The leucine 110 to glycine mutation proved to be more disruptive; the inventors were not able to measure the enthalpy change nor the affinity towards the tetra acetylated peptide at the conditions tested. Since functionality is a crucial characteristic that mutants must maintain in the bump and hole approach, Brd2(1) (Leu110-Gly) was not pursued further. Similar results were observed for the valine to glycine mutant Brd2(2) (Val376-Gly) which was therefore also not pursued. The largest decrease in binding affinity was observed for the Brd2(1) (Val103-Ala) mutant, which showed a twenty four-fold loss of affinity towards the peptide compared to the wild type construct.

### Bump Design

iBET analogues functionalized at positions R1-R7 were designed in silico to specifically target the engineered pockets in Brd2 bromodomains (FIG. 3). The iBET scaffold was selected as the starting point for ligand design due to its higher synthetic tractability and better suitability to required vectors than JQ1. It was envisaged that a “bump” originating from the methoxyphenyl ring could target a “hole” introduced onto valine 87; that functionalization at either the benzodiazepine ternary centre or at the level of the side chain methylene could target a mutation on leucine 94; and that the p-chlorophenyl ring could provide suitable vectors to explore mutations at tryptophan 81.

Docking studies using Glide [9] suggested promising substitutions to be at R1 for targeting the Val87 mutant, R3 in a (R)-configuration for targeting the Leu94 mutant, and R4-R7 for targeting the Trp81 mutant. As a starting point, a methyl group was elected as the hydrophobic “bump” of choice to explore the engineered “holes” of the mutants while introducing minimal alteration of the initial ligand scaffold in terms of charge distribution and physicochemical properties. The inventors therefore performed a “methyl scan” around the iBET scaffold by synthesizing iBET analogues functionalized with methyl groups at R1-R7 to target mutations at Trp81, Val87 and Leu94. The methyl derivatives are shown in FIG. 3, with synthetic routes to compounds 4, 5-7 and 8-10 shown in FIGS. 4-6 respectively and preliminary DSF data shown in Table 1.

TABLE 1
“Methyl scan”. Thermal stabilisation (° C.) of wild type and mutant Brd2 by iBET
derivatives, as assessed by DSF.

| brd | iBET (1) | 4 | 5 | 6 | 7 | 8 | 9 | 10 |
|---|---|---|---|---|---|---|---|---|
| brd2(1) | 5.4 ± 0.5 | 0.7 ± 0.2 | 2.2 ± 0.3 | −0.3 ± 0.2 | 3.2 ± 0.2 | 6.3 ± 0.1 | 1.5 ± 0.2 | 1.8 ± 0.2 |
| brd2(1)_{V103A} | 0.1 ± 0.6 | 0.5 ± 0.3 | / | / | / | / | / | / |
| brd2(1)_{L110I} | 6.7 ± 0.4 | / | 3.3 ± 0.4 | 0.0 ± 0.5 | 5.7 ± 0.7 | / | / | / |
| brd2(1)_{L110A} | 3.1 ± 0.4 | / | 2.9 ± 0.4 | 1.6 ± 0.2 | 7.9 ± 0.2 | / | / | / |
| brd2(1)_{W097F} | 0.4 ± 0.2 | / | / | / | / | 1.4 ± 0.2 | −0.1 ± 0.2 | 0.1 ± 0.2 |
| brd2(1)_{W097H} | 0.7 ± 0.2 | / | / | / | / | 0.9 ± 0.3 | 0.2 ± 0.2 | −0.4 ± 0.3 |
| brd2(2) | 8.3 ± 0.3 | 4.0 ± 0.1 | 5.3 ± 0.3 | 0.2 ± 0.2 | 5.6 ± 0.1 | 6.6 ± 0.2 | 3.2 ± 0.1 | 3.5 ± 0.1 |
| brd2(2)_{V376A} | 1.1 ± 0.0 | 1.2 ± 0.1 | / | / | / | / | / | / |
| brd2(2)_{V383I} | 9.3 ± 0.3 | / | 6.8 ± 0.1 | 0.3 ± 0.2 | 9.6 ± 0.1 | / | / | / |
| brd2(2_{)L383A} | 6.4 ± 0.2 | / | 6.6 ± 0.4 | 0.8 ± 0.6 | 9.3 ± 0.2 | / | / | / |
| brd2(2)_{W370F} | 2.1 ± 0.0 | / | / | / | / | 2.8 ± 0.1 | 1.5 ± 0.0 | 0.6 ± 0.1 |
| brd2(2)_{W370H} | 1.7 ± 0.2 | / | / | / | / | 1.1 ± 0.2 | 1.0 ± 0.3 | −0.1 ± 0.1 |

Introduction of methyl “bumps” at R1, R2 and R4-R6 did not provide noticeable thermal stabilisation of mutated Brd2 proteins (Table 1). In contrast, the methyl “bump” at R3 provided the first significant source of selective stabilization in the engineered system, consistent with the initial docking predictions. Indeed, alpha-methylated ester compound 7 with a (SR) configuration induced a 5.7° C. and 9.6° C. thermal stabilization of Brd2(1) Leu110-Ile and Brd2(2) Leu383-Ile, respectively, while stabilizing the respective wild-type proteins by only 3.2° C. and 5.6° C. This selective thermal stabilization was even more pronounced in the case of the Leucine-Alanine mutations, with thermal shifts of 7.9° C. and 9.3° C. against Brd2(1) Leu110-Ala and Brd2(2) Leu383-Ala, respectively. In contrast, the methyl group of compound 6 (+−)-(SS) induced no significant stabilization of the mutant proteins relative to wild-type as expected.

To validate the promising selectivity profile observed for compound 7 the inventors determined binding affinities using ITC and solved liganded X-ray crystal structures (not shown). Compound 7 displayed Kds of 1.47 μM and 300 nM against wild type Brd2(1) and Brd2(2) respectively, highlighting the destabilizing steric clash expected from the introduced methyl “bump” against wild type proteins. In contrast, 7 displayed Kds of 260 nM and 27 nM against Brd2(1) Leu110-Ile and Brd2(2) Leu383-Ile. Finally, the inventors measured Kds of 17 and 22 nM for compound 7 against Brd2(1) Leu110-Ala and Brd2(2) Leu383-Ala respectively, confirming a significant improvement in binding affinity, consistent with DSF data.

The crystal structures of Brd2(2) Leu383-Ala apo and in complex with compound 7 were subsequently solved by X-ray crystallography, at 1.5 Å and 1.7 Å resolutions, respectively. The binding mode was unambiguously assigned, and confirmed the expected positioning of the methyl substituent of the ligand within the engineered hydrophobic pocket. Noticeably, some local backbone rearrangement of the ZA loop was observed in the apo structure consistent with the known flexibility of this region.

With these results in hand the inventors designed a number of iBET and PFI-1 derivatives as potential ligands for the Brd2(1) and Brd2(2), valine, tryptophan, methionine and leucine mutants described previously and their binding to the mutant and wild type proteins evaluated.

### Valine Mutants

To test whether these new compounds bind with high affinity and selectivity towards the valine mutants, the inventors measured the thermal stabilization of these proteins and their wild types upon addition of the ligands (see Table 2).

DSF against iBET was included, to compare how much the bump enhances or weakens stability of the protein compared to the original compound. For Brd2(1) the presence of the bump in both compound 4 and compound 19 weakens the thermal affinity achieved with iBET to shifts smaller than 1° C. Although there seems to be an increase in affinity for Brd2(1)*(V103A) with the new bumped ligands compared to iBET, the shifts are very small, suggesting that affinity is not improved significantly. In the case of Brd2(2) the inventors observed a reduction of the thermal stabilization as compared to iBET; nevertheless, the bumped ligands still show a shift of about 4° C., which indicates that these molecules continue to bind this wild type with high affinity. For Brd2(2)*(Val376Ala) the inventors observed a similar result as with the valine mutation in Brd2(1), although the thermal shift is increased compared to the original compound, it is not a significant change.

At this point of the project, the valine mutants were not selected for further experimentation. The DSF results suggested that compound 4 and 19 neither bound the mutants with high affinity nor did they demonstrate high selectivity between mutants and wild types. Furthermore; initial characterization had shown that the valine mutants were the least stable and the least functional, with low melting temperatures (ΔTm from −4 to −5° C. relative to wild type—Table A) and a twenty-four-fold loss of affinity towards the tetra acetylated peptide.

TABLE 2
DSF data for compounds 4 and 19 against mutants and wild types of Brd2.

| | Brd2(1) | Brd2(1)*(Val103-Ala) | Brd2(2) | Brd(2)*(Val376-Ala) |
|---|---|---|---|---|
| | ΔTm (K) | ΔTm (K) | ΔTm (K) | ΔTm (K) |
| 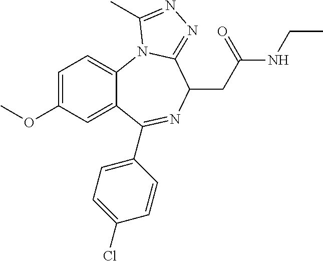 [SMILES: CCNC(=O)CC1N=C(c2ccc(Cl)cc2)c2cc(OC)ccc2-n2c(C)nnc21] iBET | 5.4 ± 0.5 | 0.1 ± 0.6 | 8.3 ± 0.3 | 1.1 ± 0.0 |
| 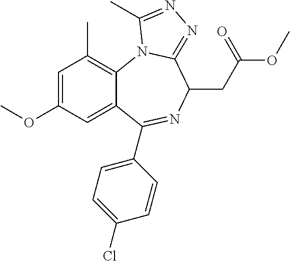 Compound 4 | 0.7 ± 0.2 | 0.5 ± 0.3 | 4.0 ± 0.1 | 1.2 ± 0.1 |
| 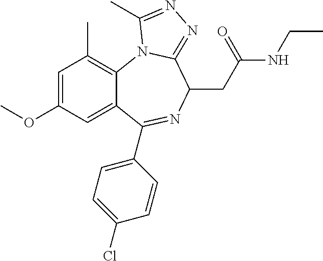 [SMILES: CCNC(=O)CC1N=C(c2ccc(Cl)cc2)c2cc(OC)cc(C)c2-n2c(C)nnc21] Compound 19 | 0.5 ± 0.2 | 0.4 ± 0.3 | 3.8 ± 0.1 | 1.3 ± 0.1 |

### Tryptophan Mutants

The tryptophan mutation was targeted by analogs of both iBET and PFI. Two different mutations were introduced instead of the tryptophan, a phenylalanine (Brd2(1)*(Trp097Phe) and Brd2(2)*(Trp370Phe) and a histidine (Brd2(1)*(Trp097His) and Brd2(2)*(Trp370His)).

Table 3 (rows 1-5) shows the results obtained with the analogs of PFI against the wild types and tryptophan mutants of Brd2. Results for these compounds were not very encouraging; none of them improved affinity towards mutants when compared to the original compound PFI. Compound 42 showed some potential for selectivity between Brd2(1) and the Brd2(2) mutant Trp370Phe. Nevertheless, overall shifts were low suggesting low affinities for both mutants and wild types. Compound 43 showed some selectivity between the wild type of Brd2(2) and its mutant Trp370Phe; however, it showed low affinity towards the other mutants. Compound 44 showed similar affinities across wild types and mutants and not very clear selectivity. Compound 45 showed the highest affinity towards the wild types but showed low affinity towards mutants. The thermal shift of Trp097Phe upon addition of compound 45 was not determined. None of these compounds were selected for ITC experiments.

Table 3 (rows 6-10) also shows the results obtained from the DSF assay with the analogs of iBET. The inventors observed that compound 9, compound 10 and compound 17 retain a certain affinity towards the wild types but do not stabilize the mutants significantly. For this reason, these molecules were not selected for further experimentation. Compound 8 and compound 18 showed an interesting behaviour, maintaining or increasing the thermal shifts obtained with iBET for the wild types and increasing the stability of all four tryptophan mutants. To study these results further, reverse titrations were performed by ITC of Brd2(2) and Brd2(2)*(Trp370Phe) (at 200 μM) into compound 18 (at 20 μM) at 25° C. Results can be seen in FIG. 7. The results showed that the Kd of the interaction between the wild type and compound 18 was around 90 nM, while the Kd for the mutant with this compound was around 300 nM. Although these results were interesting, they did not demonstrate selectivity for the mutant versus the wild type bromodomain protein.

TABLE 3
DSF data for analogs of PFI targeting wild types and tryptophan mutants of
BRD2.

| | | Brd2(1)* | | | Brd2(2)* | |
|---|---|---|---|---|---|---|
| | Brd2(1) | (Tryp097- | Brd2(1)* | Brd2(2) | (Tryp370- | Brd2(2)* |
| | ΔTm | Phe) ΔTm | (Tryp097- | ΔTm | Phe) | (Tryp370-His) |
| | (K) | (K) | His) ΔTm (K) | (K) | ΔTm (K) | ΔTm (K) |
| 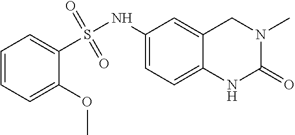 [SMILES: COc1ccccc1S(=O)(=O)Nc1ccc2c(c1)CN(C)C(=O)N2] PFI-1 | 4.5 ± 0.3 | 2.6 ± 0.4 | 1.9 ± 0.5 | 4.5 ± 0.2 | 2.9 ± 0.3 | 1.8 ± 0.0 |
| 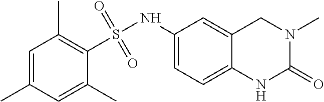 [SMILES: Cc1cc(C)c(S(=O)(=O)Nc2ccc3c(c2)CN(C)C(=O)N3)c(C)c1] Compound 42 | 0.7 ± 0.3 | 0.7 ± 0.2 | 0.2 ± 0.4 | 2.7 ± 0.5 | 1.8 ± 0.2 | 0.2 ± 0.2 |
| 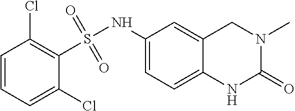 [SMILES: CN1Cc2cc(NS(=O)(=O)c3c(Cl)cccc3Cl)ccc2NC1=O] Compound 43 | 1.2 ± 0.2 | 0.2 ± 0.3 | 0.2 ± 0.2 | 0.6 ± 0.1 | 1.8 ± 0.3 | 0.3 ± 0.1 |
| 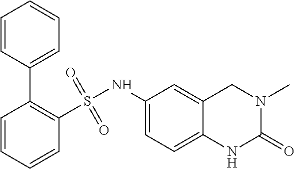 [SMILES: CN1Cc2cc(NS(=O)(=O)c3ccccc3-c3ccccc3)ccc2NC1=O] Compound 44 | 2.8 ± 0.4 | 1.8 ± 0.3 | 1.3 ± 0.2 | 2.1 ± 0.4 | 2.2 ± 0.3 | 0.5 ± 0.3 |
| 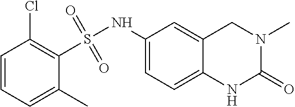 [SMILES: Cc1cccc(Cl)c1S(=O)(=O)Nc1ccc2c(c1)CN(C)C(=O)N2] Compound 45 | 3.9 ± 0.2 | n/a | 0.0 ± 0.3 | 5.3 ± 0.5 | 2.3 ± 0.1 | 0.8 ± 0.1 |
| 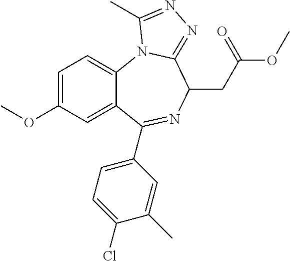 Compound 9 | 1.5 ± 0.2 | −0.1 ± 0.2 | 0.2 ± 0.2 | 3.2 ± 0.1 | 1.5 ± 0.0 | 1.0 ± 0.3 |
| 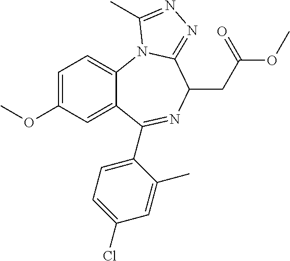 Compound 8 | 6.3 ± 0.1 | 1.4 ± 0.2 | 0.9 ± 0.3 | 6.6 ± 0.2 | 2.8 ± 0.1 | 1.1 ± 0.2 |
| 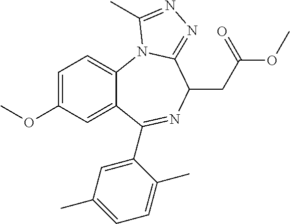 Compound 10 | 1.8 ± 0.2 | 0.1 ± 0.2 | −0.4 ± 0.3 | 3.5 ± 0.1 | 0.6 ± 0.1 | −0.1 ± 0.1 |
| 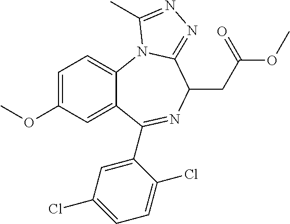 Compound 17 | 1.2 ± 0.2 | 0.0 ± 0.2 | −0.4 ± 0.2 | 2.5 ± 0.2 | 0.3 ± 0.0 | −0.4 ± 0.2 |
| 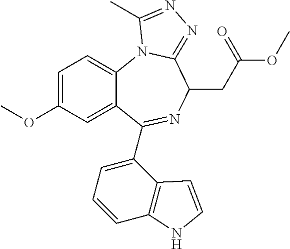 Compound 18 | 6.8 ± 0.6 | 1.9 ± 0.5 | 0.6 ± 0.3 | 7.7 ± 0.2 | 5.1 ± 0.1 | 2.7 ± 0.4 |

Methionine Mutants For these experiments, two different point mutations were introduced instead of a methionine residue, either a leucine or an alanine residue in both domains of Brd2. An important challenge with PFI analogs was their lower solubility, which in some cases hindered DSF measurements. Results can be seen in Table 4. Compound 47, compound 48 and compound 53 showed small or no stabilization of mutant proteins. In the case of compound 53 the low solubility of the compound could be responsible for the values obtained. Compound 49, compound 52 and compound 54 showed similar shifts across wild types and mutants, suggesting poor selectivity of these compounds. Compound 46, compound 50 and compound 51 showed very promising results. Compound 46 was the first molecule that showed a pattern closer to what the inventors were aiming for with the bump and hole approach: low affinity towards wild types (small thermal shifts) and higher affinity towards a mutant (larger thermal shifts). By increasing the size of the bump in compound 46, the inventors expected to see even higher selectivity.

This was found to be the case for compound 50, which successfully increased the affinity of the bumped ligands against the mutants, as well as the selectivity between mutants and wild types. However, solubility of the compound decreased, which affected the thermal shift measurement. Compound 51 showed the best results from this batch of molecules, with ΔTms between 1-2° C. for the wild types and 4.5-6.6° C. for the methionine to alanine mutants. Furthermore; this compound showed good solubility and its small size can still be exploited by adding bigger bumps that could potentially increase affinity and selectivity.

TABLE 4
DSF data for analogs of PFI targeting wild types and methionine mutants of
BRD2.

| | | Brd2(1)* | Brd2(1)* | | Brd2(2)* | Brd2(2)* |
|---|---|---|---|---|---|---|
| | | (Met165- | (Met165- | | (Met165- | (Met165- |
| | Brd2(1) | Ala) ΔTm | Leu) ΔTm | Brd2(2) | Ala) | Leu) |
| | ΔTm (K) | (K) | (K) | ΔTm (K) | ΔTm (K) | ΔTm (K) |
| 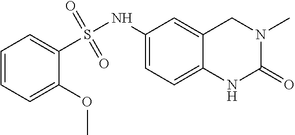 [SMILES: COc1ccccc1S(=O)(=O)Nc1ccc2c(c1)CN(C)C(=O)N2] PFI-1 | 4.5 ± 0.3 | 1.6 ± 0.1 | 1.3 ± 0.3 | 4.5 ± 0.2 | 1.7 ± 0.1 | 2.1 ± 0.3 |
| 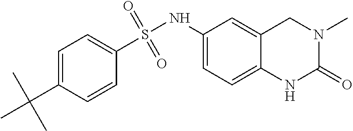 [SMILES: CN1Cc2cc(NS(=O)(=O)c3ccc(C(C)(C)C)cc3)ccc2NC1=O] Compound 46 | 1.1 ± 0.2 | 5.1 ± 0.2 | 0.1 ± 0.1 | 0.8 ± 0.1 | 2.5 ± 0.2 | 0.3 ± 0.2 |
| 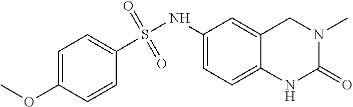 [SMILES: COc1ccc(S(=O)(=O)Nc2ccc3c(c2)CN(C)C(=O)N3)cc1] Compound 47 | 2.4 ± 0.1 | 0.0 ± 0.3 | 0.1 ± 0.3 | 3.1 ± 0.4 | 1.6 ± 0.3 | 1.3 ± 0.2 |
| 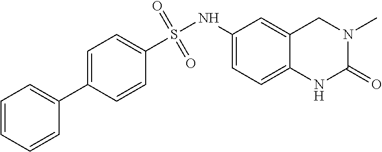 [SMILES: CN1Cc2cc(NS(=O)(=O)c3ccc(-c4ccccc4)cc3)ccc2NC1=O] Compound 48 | 0.5 ± 0.2 | 1.5 ± 0.4 | 0.0 ± 0.1 | 0.0 ± 0.2 | 0.0 ± 0.1 | 0.0 ± 0.1 |
| 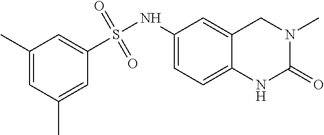 [SMILES: Cc1cc(C)cc(S(=O)(=O)Nc2ccc3c(c2)CN(C)C(=O)N3)c1] Compound 49 | 2.6 ± 0.5 | 1.1 ± 0.5 | 4.1 ± 0.6 | 2.8 ± 0.2 | 2.5 ± 0.2 | 4.9 ± 0.3 |
| 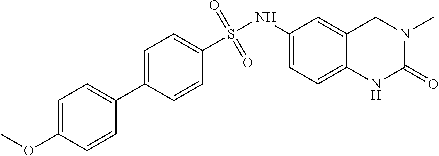 [SMILES: COc1ccc(-c2ccc(S(=O)(=O)Nc3ccc4c(c3)CN(C)C(=O)N4)cc2)cc1] Compound 50 | n/a | 5.3 ± 0.3 | 1.7 ± 0.1 | 0.7 ± 0.2 | 4.1 ± 0.1 | 3.8 ± 0.2 |
| 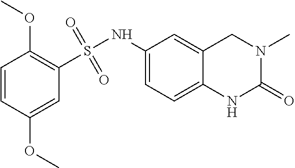 [SMILES: COc1ccc(OC)c(S(=O)(=O)Nc2ccc3c(c2)CN(C)C(=O)N3)c1] Compound 51 | 1.1 ± 0.2 | 4.6 ± 0.2 | 2.9 ± 0.2 | 2.1 ± 0.3 | 6.6 ± 0.3 | 5.2 ± 0.3 |
| 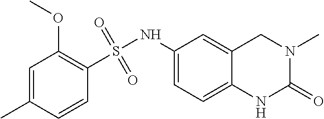 [SMILES: COc1cc(C)ccc1S(=O)(=O)Nc1ccc2c(c1)CN(C)C(=O)N2] Compound 52 | 4.7 ± 0.2 | 1.2 ± 0.2 | 3.1 ± 0.2 | 2.8 ± 0.2 | 2.0 ± 0.2 | 2.4 ± 0.1 |
| 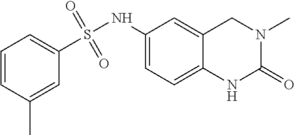 [SMILES: Cc1cccc(S(=O)(=O)Nc2ccc3c(c2)CN(C)C(=O)N3)c1] Compound 53 | 0.0 ± 0.3 | 0.0 ± 0.1 | 0.0 ± 0.1 | 0.7 ± 0.1 | 0.0 ± 0.1 | 0.0 ± 0.0 |
| 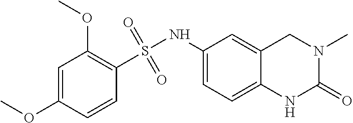 [SMILES: COc1ccc(S(=O)(=O)Nc2ccc3c(c2)CN(C)C(=O)N3)c(OC)c1] Compound 54 | 2.4 ± 0.2 | 1.5 ± 0.2 | 2.7 ± 0.3 | 1.6 ± 0.2 | 2.3 ± 0.1 | 2.4 ± 0.4 |

To confirm these results, compound 51 was selected for ITC. The curves can be seen in FIG. 8. The Kd for Brd2(1)+compound 51 was around 3.2 μM, while the Kd for Brd2(1)*(Met165Ala) upon addition of the same compound was around 500 nM resulting in a six-fold selectivity. For Brd2(2)+compound 51 the inventors measured a Kd of about 1.6 μM while the Kd for the methionine to alanine mutation of this wild type with compound 51 was around 260 nM—also a six fold selectivity for the mutant protein relative to the wild type.

Ultimately, the goal of the project is to develop a tool with which the inventors can modulate one of the domains within a tandem BET bromodomain containing protein. With compound 51 the inventors were able to achieve a twelve fold higher affinity towards Brd2(2)*(Met165Ala) compared to the wild type Brd2(1). With the same compound the inventors achieved a three-fold higher affinity towards Brd2(1)*(Met438Ala) than towards the wild type of Brd2(2). These results were very encouraging and the small size of the compounds as well as the large hole produced by mutation of methionine to alanine leave room for improvement.

### Leucine Mutants

Two different mutants were produced for each Brd2 domain, with a leucine to alanine mutation or a leucine to isoleucine mutation. The DSF results are shown in Table 5. From the DSF data the inventors concluded that compound 6 was an inactive diastereomer with no stabilization effect for the wild types and only small shifts for the leucine to alanine mutants. The inventors also observed that compound 5 reduced the thermal stabilization achieved by iBET for the wild types and the leucine to isoleucine mutants by about 3° C., while the leucine to alanine mutants retained a similar thermal shift as with iBET.

While these results were interesting, the results obtained with compound 7 were the most promising results obtained to this point for the bump and hole approach. From Table 5 it is clear that the presence of the bump in compounds 5-7 reduces the stabilization of the wild types compared to IBET (cf. ΔTm values for Brd2(1) and Brd2(2) wild type). In contrast, the presence of the bump in compound 7 only but not compounds 5 or 6 significantly increased stabilisation of the Leu-Ala mutants relative to wild type proteins.

TABLE 5
DSF data for analogs of iBET targeting wild types and leucine mutants of Brd2.

| | | Brd2(1)* | Brd2(1)* | | Brd2(2)* | Brd2(2)* |
|---|---|---|---|---|---|---|
| | | (Leu110- | (Leu110- | | (Leu383- | (Leu383- |
| | Brd2(1) | Ala) ΔTm | Ile) ΔTm | Brd2(2) | Ala) | Ile) |
| | ΔTm (K) | (K) | (K) | ΔTm (K) | ΔTm (K) | ΔTm (K) |
| 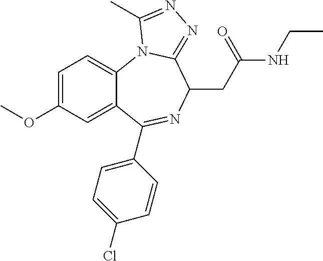 [SMILES: CCNC(=O)CC1N=C(c2ccc(Cl)cc2)c2cc(OC)ccc2-n2c(C)nnc21] iBET | 5.4 ± 0.5 | 3.1 ± 0.4 | 6.7 ± 0.4 | 8.3 ± 0.3 | 6.4 ± 0.2 | 9.3 ± 0.3 |
| 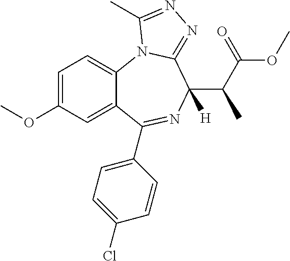 Compound 6 | −0.3 ± 0.2 | 1.6 ± 0.2 | 0.0 ± 0.5 | 0.2 ± 0.2 | 0.8 ± 0.6 | 0.3 ± 0.2 |
| 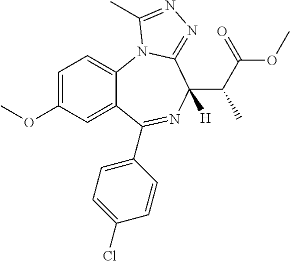 Compound 7 | 3.2 ± 0.2 | 7.9 ± 0.2 | 5.7 ± 0.7 | 5.6 ± 0.1 | 9.3 ± 0.2 | 9.6 ± 0.1 |
| 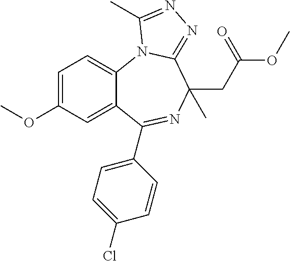 Compound 5 | 2.2 ± 0.3 | 2.9 ± 0.4 | 3.3 ± 0.3 | 5.3 ± 0.3 | 6.6 ± 0.4 | 6.8 ± 0.1 |

A similar trend was observed in the Leu-Ile mutants, with compound 7 significantly increasing stability of the mutants relative to the wild type proteins. To quantify the difference in affinity between the wild types and the leucine mutants towards compound 7, reverse titrations were performed by ITC. Leucine mutants were titrated at a concentration of 200 μM into a solution of 20 μM 7 at 25° C. Expecting a lower Kd, wild types were titrated at a concentration of 350 μM into a solution of 20 μM 7 at 25° C.

The ITC curves and results are shown in FIG. 9. If we take only the leucine to alanine mutants into account, we can observe, that the wild type of Brd2(2) maintains a high affinity towards compound 7. For this reason, the affinity of this compound towards Brd2(1)*(Leu110-Ala) is only eighteen fold higher than for Brd2(2). On the other hand, we can observe that compound 7 is an effective tool to modulate Brd2(2) individually, since this compound has a sixty six fold higher affinity for Brd2(2)*(Leu383-Ala) than for the wild type of Brd2(1). Due to the results presented above, this mutant-compound pair was co-crystallized and solved. A close up of compound 7 in the binding site of Brd2(2)*(Leu383-Ala) shows that the added bump points directly into the leucine to alanine mutation. Furthermore, the crystal structure (see reference 15) suggests that the hole is large enough to support a larger bump. The promising results obtained by DSF, ITC and crystallization were crucial for selecting the leucine to alanine mutants+compound 7 for further optimisation.

### Summary

Novel chemical probes based on the iBET and PFI-1 bromodomain inhibitors were screened against the wild type BET-Brd proteins and the biophysically characterised mutants. Two series of probes were screened; a series based on the PFI-1 scaffold and a series based on the iBET scaffold.

Different subsets of two compound series (compounds 4-19 and 42-53) were screened against the tryptophan, valine and leucine mutants. Compounds 42-53 were screened against the tryptophan and methionine mutants, since the PFI-1 scaffold possesses suitable vectors to target these mutations.

The methionine to alanine and methionine to leucine mutants were screened solely against analogs of PFI. Three compounds in particular, compound 46, compound 50 and compound 51, displayed the required properties of low affinity for the wild type vs. high affinity for the mutants. A twelve fold higher affinity of Brd2(1)*(Met165-Ala) towards compound 51 was observed compared to that of the wild type of Brd2(2), although the same compound only exhibited a three-fold higher affinity towards Brd2(2)*(Met438-Ala) than towards the wild type of Brd2(1). Nonetheless; these results were very encouraging.

The most promising results for the iBET analogues were obtained with the leucine/alanine and leucine/isoleucine mutants. Compound 7 was found to exhibit eighteen fold selectivity for Brd2(1)*(Leu110-Ala) over the wild type Brd2(2) and sixty six fold selectivity for Brd2(2)*(Leu383-Ala) over the wild type of Brd2(1). Accordingly compound 7 was co-crystallized with Brd2(2)*(Leu383-Ala) for detailed structural analysis. Mutants of all eight single BET bromodomains were prepared containing the leucine to alanine mutation in the same position within the binding site. The inventors also engineered three tandem constructs of Brd2 containing either one mutation in only one bromodomain or a leucine to alanine mutation on both bromodomains. All these constructs and all the wild type proteins were expressed and purified to complete eight individual wild type BET bromodomains, eight individual mutant BET bromodomains containing the leucine to alanine mutation and four tandem constructs of Brd2.

Importantly, the above results confirm that the bump and hole technique represents a promising approach for targeting individual domains within a population of domains of similar structure.

Based on the above results, compound 7 was chosen for additional optimisation to improve selectivity of the system, by maintaining high affinity towards the mutants and weakening interaction between wild types and novel compounds and also to translate any positive results across the whole BET bromodomain subfamily.

To tackle these goals, an array of molecules based on 7 but with longer bumps were synthesised (compounds 11-13). Each one of these molecules also had an inactive diastereomer (compounds 14-16) which were tested by DSF to verify inactivity (Table 6).

TABLE 6
DSF data for inactive diastereomers targeting leucine mutant.

| Protein | 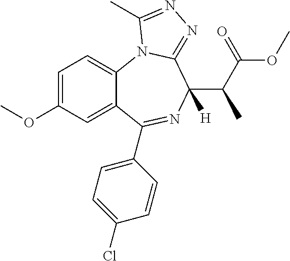 ΔTm + Compound 6 [K] | 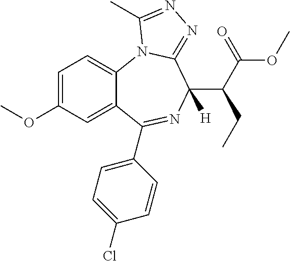 ΔTm + Compound 14 [K] | 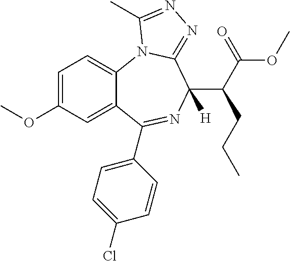 ΔTm + Compound 15 [K] | 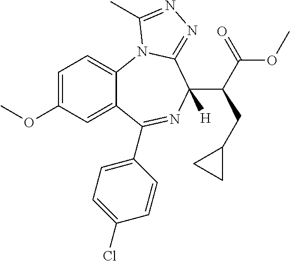 ΔTm + Compound 16 [K] |
|---|---|---|---|---|
| Brd2(1) | −0.3 ± 0.2 | 1.0 ± 0.3 | 0.1 ± 0.3 | −0.5 ± 0.6 |
| Brd2(1)* | 1.6 ± 0.2 | 1.8 ± 0.4 | 0.9 ± 0.6 | 1.6 ± 1.0 |
| (L110A) | | | | |
| Brd2* | 0.0 ± 0.5 | 0.5 ± 0.0 | / | / |
| (L1101) | | | | |
| Brd2(2) | 0.2 ± 0.2 | 0.9 ± 0.4 | 0.5 ± 0.2 | 1.1 ± 0.3 |
| Brd2(2)* | 0.8 ± 0.6 | 2.4 ± 0.6 | 1.4 ± 0.2 | 2.3 ± 0.3 |
| (L383A) | | | | |
| Brd2(2)* | 0.3 ± 0.2 | 0.9 ± 0.3 | / | / |
| (L383I) | | | | |
| Brd4(1) | 0.0 ± 0.1 | 0.5 ± 0.1 | 0.6 ± 1.1 | −0.4 ± 0.5 |
| Brd4(1)* | / | / | 2.4 ± 1.3 | 1.3 ± 0.2 |
| (L094A) | | | | |
| Brd4(2) | 0.1 ± 0.1 | 0.1 ± 0.1 | −0.5 ± 1.1 | 0.3 ± 0.9 |
| Brd4(2)* | / | / | 1.6 ± 0.3 | 0.1 ± 0.1 |
| *L387A) | | | | |

In addition, new mutants were constructed in which the leucine to alanine mutation was introduced into all the members of the BET subfamily via site directed mutagenesis (SDM). The leucine to alanine mutation was also introduced via SDM in a tandem construct of Brd2 containing the first and the second bromodomain, as well as the natural linker between them. Four constructs were expressed and purified containing either one mutation in one of the domains, the leucine to alanine mutation on both domains or no mutations at all. Thermal stabilization for both wild type constructs of Brd2 and Brd4, as well as their respective leucine to alanine mutants are shown in Table 7.

From the DSF data we can see that affinity towards wild types decreases, when going from a methyl bump in compound 7 to an ethyl bump in compound 11. However; subsequent elongation of the bump in compounds 12 and 13 does not destabilize the interaction between wild types and the compounds further. A possible explanation for this is the rotamers in the compounds, which would allow for the bump to point towards the solvent instead of clashing against the wild type leucine residue of the protein.

However, we do observe that the shifts of all the Leu-Ala mutants with the ethyl bump are very close to those with the methyl bump, suggesting that the ethyl bump can still be accommodated by the hole produced by the leucine to alanine mutation. In contrast, increasing the bump to a propyl and a cyclopropyl appears to weaken the interaction between the molecule and mutant proteins.

TABLE 7
DSF data for SAR of leucine targeting compounds

| | 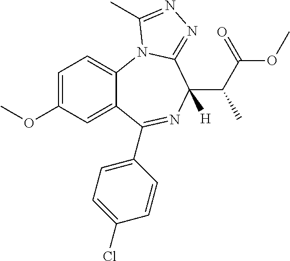 Compound 7 ΔTm [K] | 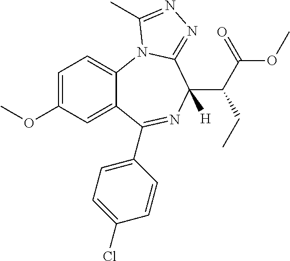 Compound 11 ΔTm [K] | 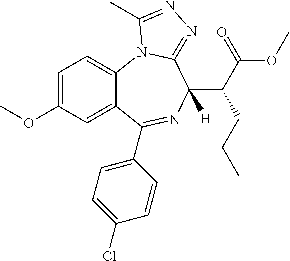 Compound 12 ΔTm [K] | 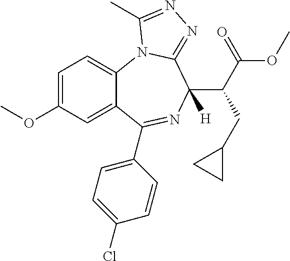 Compound 13 ΔTm [K] |
|---|---|---|---|---|
| Brd2(1) | 3.2 ± 0.2 | 1.2 ± 0.2 | 1.7 ± 0.2 | 0.5 ± 0.3 |
| Brd2(1)* (L110A) | 7.9 ± 0.2 | 7.6 ± 0.2 | 4.0 ± 0.4 | 3.7 ± 0.2 |
| Brd2(2) | 5.6 ± 0.1 | 1.6 ± 0.1 | 2.1 ± 0.1 | 3.7 ± 0.6 |
| Brd2(2)* (L383A) | 9.3 ± 0.2 | 8.1 ± 0.2 | 5.0 ± 0.1 | 5.3 ± 0.3 |
| Brd4(1) | 4.0 ± 0.4 | 1.5 ± 0.4 | 2.9 ± 0.7 | 1.2 ± 0.6 |
| Brd4(1)* (L094A) | 10.7 ± 0.8 | 10.0 ± 0.2 | 6.4 ± 0.8 | 5.6 ± 0.3 |
| Brd4(2) | 4.7 ± 0.0 | 1.5 ± 0.0 | 3.2 ± 0.7 | 2.0 ± 1.0 |
| Brd4(2)* (L387A) | 8.4 ± 0.1 | 7.0 ± 0.1 | 6.7 ± 0.2 | 5.8 ± 0.1 |

To investigate this further, the inventors performed reverse titrations by ITC of Brd2 proteins into all four molecules. Experiments with compounds 7 and 11 were performed at 25° C., these compounds were dissolved in ethanol. Solubility of compounds 12 and 13 was lower than that for compounds with smaller bumps; for this reason, compounds were dissolved in DMSO and ITC experiments were run at 30° C. and 1% DMSO in both the cell and the syringe. The results can be seen in Table 8.

TABLE 8
ITC data for SAR of leucine targeting compounds.

| | Compound 7 (25° C.) | | Compound 11 (25° C.) | |
|---|---|---|---|---|
| Protein | Kd [nM] | ΔH [cal/mol] | Kd [nM] | ΔH [cal/mol] |
| Brd2(1) | 1470 ± 200 | −8653 ± 135.7 | 1780 ± 2000 | −2957 ± 214.4 |
| Brd2(1)* (Leu110-Ala) | 17.0 ± 5.5 | −16780 ± 161.4 | 42.7 ± 7.8 | −16220 ± 130.9 |
| Brd2(2) | 298.5 ± 114.2 | −5360 ± 117.0 | 2200 ± 400 | −3601 ± 85.2 |
| Brd2(2)* (Leu383-Ala) | 22.3 ± 4.5 | −12610 ± 86.06 | 21.7 ± 5.6 | −10710 ± 102.8 |
| | Compound 12 (30° C.) | | Compound 13 (30° C.) | |
| Protein | Kd [nM] | ΔH [cal/mol] | Kd [nM] | ΔH [cal/mol] |
| Brd2(1) | 5882 ± 1215 | −11490 ± 1048 | 4310 ± 3053 | −3402 ± 433.5 |
| Brd2(1)* (Leu110-Ala) | 400 ± 82.4 | −11160 ± 241.8 | 360 ± 68.2 | −9662 ± 162.7 |
| Brd2(2) | 2392 ± 1483 | −9520 ± 544.9 | 3322 ± 1859 | −2089 ± 211.0 |
| Brd2(2)* (Leu383-Ala) | 264.6 ± 53.9 | −7095 ± 106.9 | 667 ± 330 | −7868 ± 336.5 |

The results obtained by ITC mirror what was observed in the DSF analysis. Compound 11 is the clear stand out between the array of molecules; showing not only high affinity towards the mutants but also higher selectivity for mutants relative to wild types. This is reflected not only in the Kds, but also in the enthalpy changes. With this molecule-mutant pair, the optimization at this point was achieved and the next step was to study if the obtained results were translatable throughout the whole BET subfamily. To this end, DSF data was collected for all eight wild types and all eight leucine to alanine mutants of the BET subfamily. Results can also be seen in FIG. 10, with curves obtained for Brd4(1) and the respective leucine to alanine mutant shown as an example.

Results obtained were very promising, showing small shifts from 1.2° C. to a maximum of 2.6° C. for all the eight BET wild types and shifts from 5.4° C. to 13.4° C. for all the leucine to alanine mutants. The leucine to alanine mutants of Brdt showed smaller shifts than the rest of the mutants; however, the shifts are significantly higher than those obtained with iBET for these mutants (ΔTm values of 0.9° C. and 3.0° C.—see Table 9). DSF results for all wild types and mutants against iBET, compound 7 and compound 11, as well as melting temperatures for all constructs are shown in Table 9. To quantify the affinities of all the bromodomain constructs towards compound 11, reverse titrations were performed by ITC at 30° C. and 1% DMSO. Results are shown in FIG. 12.

TABLE 9
DSF data for all wild types and leucine to alanine mutants

| | | | ΔTm + | ΔTm + |
|---|---|---|---|---|
| | | ΔTm + | Compound 7 | Compound |
| Protein | Tm [° C.] | iBET [K] | [K] | 11 [K] |
| Brd2(1) | 46.0 ± 0.1 | 5.4 ± 0.5 | 3.2 ± 0.2 | 1.2 ± 0.2 |
| Brd2(1)* (L110A) | 43.7 ± 0.0 | 3.1 ± 0.4 | 7.9 ± 0.2 | 7.6 ± 0.2 |
| Brd2(2) | 47.5 ± 0.1 | 8.3 ± 0.3 | 5.6 ± 0.1 | 1.6 ± 0.1 |
| Brd2(2)* (L383A) | 44.8 ± 0.1 | 6.4 ± 0.2 | 9.3 ± 0.2 | 8.1 ± 0.2 |
| Brd3(1) | 46.4 ± 0.1 | 7.1 ± 0.1 | 5.9 ± 0.2 | 2.2 ± 0.1 |
| Brd3(1)* (L070A) | 44.8 ± 0.1 | 3.7 ± 0.2 | 10.8 ± 0.3 | 10.4 ± 0.3 |
| Brd3(2) | 41.5 ± 0.6 | 8.5 ± 0.7 | 6.7 ± 1.0 | 2.6 ± 0.7 |
| Brd3(2)* (L344A) | 40.5 ± 0.1 | 8.3 ± 0.3 | 15.6 ± 0.4 | 13.4 ± 0.6 |
| Brd4(1) | 44.6 ± 0.2 | 8.0 ± 0.5 | 4.0 ± 0.4 | 1.5 ± 0.4 |
| Brd4(1)* (L094A) | 44.8 ± 0.1 | 5.6 ± 0.2 | 10.7 ± 0.8 | 10.0 ± 0.2 |
| Brd4(2) | 44.7 ± 0.0 | 7.8 ± 0.7 | 4.7 ± 0.0 | 1.5 ± 0.0 |
| Brd4(2)* (L387A) | 44.8 ± 0.1 | 7.5 ± 0.1 | 8.4 ± 0.1 | 7.0 ± 0.1 |
| Brdt(1) | 48.6 ± 0.3 | 7.1 ± 0.5 | 5.3 ± 0.3 | 1.5 ± 0.4 |
| Brdt(1)* (L063A) | 47.4 ± 0.2 | 0.9 ± 0.3 | 5.8 ± 0.3 | 5.4 ± 0.3 |
| Brdt(2) | 44.4 ± 0.2 | 5.4 ± 0.3 | 3.7 ± 0.3 | 1.3 ± 0.3 |
| Brdt(2)* (L306A) | 45.3 ± 0.2 | 3.0 ± 0.5 | 7.9 ± 0.2 | 6.7 ± 0.4 |

For all mutants, protein was titrated at a concentration between 150-200 μM into a solution of the compound at a concentration of 15-20 μM. In the case of the wild types, protein was titrated at a concentration of 350-400 μM into a solution of the compound between 15-20 μM. FIG. 11 is an illustrative example of the curves obtained for the Brd3(1) bromodomain construct and its respective leucine to alanine mutant titrated into compound 11. We can easily observe a hyperbolic shape for the titration of wild type Brd3(1) into compound 11, corresponding to a low affinity interaction, while for Brd3(1)*(Leu070-Ala) we can easily observe a sharp sigmoidal behaviour corresponding to a high affinity interaction. Furthermore, there is a clear difference in the ΔH produced by each interaction. (ΔH=−22 kcal/mol vs. mutant compared to −7 kcal/mol vs. wild type).

The rest of the BET subfamily members follow this trend, as seen in FIGS. 10 and 12.

Tandem constructs of Brd2 with and without leucine to alanine mutations were expressed and purified for this last part of the project. Expression and purification of these constructs proved to be challenging, with lower yields than the individual domains. Nevertheless, ITC experiments were performed with four of these constructs, a tandem with no mutation (WT-WT), a tandem with the mutation in the first bromodomain (LA-WT), a tandem with the mutation on the second bromodomain (WT-LA) and a tandem with mutations on both bromodomains (LA-LA). Normal titrations (compound into syringe, protein into sample cell) with compound 11 were performed for this assay. For the WT-WT construct, 300 μM of the compound was injected into a solution of the tandem at 10 μM. For the LA-WT and WT-LA constructs, the compound was injected at 150 μM into 15 μM of the protein and for the LA-LA construct, 150 μM compound was injected into a solution of 10 μM of this tandem construct. The ITC assays were run at 30° C. at 1% DMSO.

The results can be seen in FIG. 13A with a repeat of the experiment shown in FIG. 13B. For the non-mutated tandem (WT-WT) we can easily observe a hyperbolic curve, typical for interactions with low affinity. The measured Kd is about 15.8 μM, which lies between the Kd measured for the individual wild type domains of Brd2 with compound 11 shown in the previous section. For the LA-WT mutant we were able to observe a sigmoidal curve, and the measured Kd was around 160 nM. Additionally, the ΔS and ΔH values obtained from this measurement are very close to those obtained for the single Brd2(1)*(L110A) mutant. Here, we have to take into account that curves are fitted with the one site-binding model and final curves will be composed of the heat absorbed or produced in both domains of the tandem.

For the WT-LA tandem construct, the results were unexpected, the curve obtained shows a low C-value (i.e. a hyperbolic curve); for this reason, the thermodynamic parameters could only be poorly fitted, and it was not possible to measure an accurate Kd or an accurate ΔH. The low C-value is probably a consequence of the impurity of the sample, which in turn produces an overestimation of the protein concentration. Looking back at the results of the purification from the gel filtration traces and the SDS PAGE gel, the inventors were able to observe clear impurities that could have contributed to this result. Lastly, for the tandem construct containing the leucine to alanine mutation on both domains, we measured a Kd of about 56 nM which is very close to the Kds obtained for the single domains Brd2(1)*(Leu110-Ala) and Brd2(2)*(Leu383-Ala). Additionally, the ΔH and ΔS values measured lie between the values obtained for both individual domains, as we would expect for a tandem construct containing both mutated domains.

The results obtained with these tandem constructs are in agreement with the results obtained with individual domains. The inventors observed that it is possible to target an individual bromodomain by the example of the LA-VVT mutant, in which we can measure high affinity corresponding to only one domain. The results obtained for the WT-VVT and LA-LA constructs reinforce this result.

To establish whether selectivity could also be observed within cells, we developed fluorescence recovery after photobleaching (FRAP) assays using full length human GFP-BRD4 transfected into human osteosarcoma cells (U2OS). FIG. 14A shows ITC titrations of compound 11 against a WT-VVT tandem construct of Brd2 (white) and its L/A-L/A double mutant counterpart (black) at 30° C. FIG. 14B shows fluorescence recovery after photobleaching (FRAP) evaluation of the selectivity of compound 11 in U2OS cells transfected with full-length human GFP-brd4. Time-dependence of the fluorescence recovery of cells (main panel) and a quantitative comparison of half-time of fluorescence recovery (inset panel) are shown for cells expressing VVT GFP-brd4 treated with DMSO (vehicle control) or 1 μM iBET, and for cells expressing VVT or L/A-L/A GFP-brd4 treated with 1 μM 11. The data shown represent the mean±SEM (n=35-50). Statistical significance was determined by one-tailed t tests: *<0.05; **P<0.01; ***P<0.001; n.s. not significant.

Control treatment with 1 μM iBET accelerated the fluorescence recovery of the photobleached nuclear region of cells transfected with VVT GFP-Brd4 (FIG. 14), indicating displacement of BRD4 from chromatin, as expected based on previous results reported with other BET inhibitors e.g. JQ1 [10]. Crucially, treatment with 1 μM compound 11 against WT showed no reduction of recovery times relative to vehicle-treated cells (FIG. 14) however exposure of 1 μM compound 11 against a double Leu(94,387)/Ala mutant of GFP-Brd4, showed recovery times comparable to the iBET control, confirming the high selectivity of compound 11 inside cells. Taken together, our data show that small-molecule targeting of bromodomains within the BET subfamily can be achieved for the first time with exquisite control and high selectivity in vitro and in cells.

To summarise, the inventors produced mutants of all eight single BET bromodomains containing the leucine to alanine mutation in the same position within the binding site. The inventors also engineered three tandem constructs of Brd2 containing either one mutation in only one bromodomain or a leucine to alanine mutation on both bromodomains. All these constructs and all the wild type proteins were expressed and purified to complete eight individual wild type BET bromodomains, eight individual mutant BET bromodomains containing the leucine to alanine mutation and four tandem constructs of Brd2. At the same time, we synthesized three new molecules containing bulkier bumps. An SAR including DSF and ITC screening of these new compounds against wild types and mutants of Brd2s and Brd4s revealed compound 11 as the clear stand out between the array of molecules. This compound containing an ethyl bump was then screened by DSF and ITC against all the members of the BET subfamily and their respective leucine to alanine mutants. Results showed that compound 11 bound all eight BET bromodomains containing the leucine to alanine mutation with high affinity. At the same time, compound 11 showed low affinity towards the wild types of all eight BET bromodomains. Experiments with the tandem constructs are largely in agreement with the results obtained with individual domains. Results with these constructs suggest that it is possible to target an individual bromodomain within a construct containing paired bromodomains.

The selectivity factors in Table 10 below are defined by the ratio KdVVT/KdLeu/Ala, with wild type proteins being read across the top row and mutant proteins being read down the first column. As an example, compound 11 is 273 fold more potent against brd3(1)Leu070Ala than against VVT brd4(2). Future experimentation will involve mutating only one of the eight BET bromodomains to then modulate it without affecting the function of the rest of the BET bromodomains. For example; if we want to study the role of Brd2(1) we can mutate this bromodomain and we will have at least a 96 fold selectivity against all other bromodomains, which will allow us to target this bromodomain individually and independently. The lowest selectivity is found for the leucine to alanine mutation of Brd4(2), (thirty-five fold relative to Brd3(1)). Nevertheless, this is much higher than can be achieved with any other molecule published to date and fine tuning of the concentrations could allow individual modulation of this bromodomain as well.

TABLE 10
Selectivity profile for compound 11 at 30° C.

| brd | brd2(1) | brd2(2) | brd3(1) | brd3(2) | brd4(1) | brd4(2) | brdt(1) | brdt(2) |
|---|---|---|---|---|---|---|---|---|
| brd2(1)_{L110A} | 123 | 236 | 96 | 101 | 174 | 138 | 117 | 167 |
| brd2(2)_{L383A} | 105 | 203 | 82 | 87 | 150 | 118 | 101 | 144 |
| brd3(1)_{L070A} | 244 | 469 | 190 | 202 | 346 | 273 | 233 | 332 |
| brd3(2)_{L344A} | 135 | 261 | 106 | 112 | 192 | 152 | 130 | 185 |
| brd4(1)_{L094A} | 206 | 396 | 160 | 170 | 292 | 231 | 197 | 281 |
| brd4(2)_{L387A} | 45 | 88 | 35 | 38 | 65 | 51 | 44 | 62 |
| brdt(1)_{L063A} | 48 | 92 | 37 | 40 | 67 | 54 | 46 | 65 |
| brdt(2)_{L306A} | 63 | 122 | 49 | 52 | 90 | 71 | 60 | 86 |

From the results presented above, we can conclude that the hole and bump technique is a successful approach for targeting individual bromodomains. The inventors were able to identify a novel chemical probe (Compound 11) that not only shows high affinity towards a mutated BET bromodomain, but also shows high selectivity compared to the rest of the wild types. Specificity achieved towards any individual BET bromodomain is far beyond that obtained by any other currently published molecule. In addition, results were translatable throughout the whole BET subfamily, showing that we have developed a tool that allows for selective modulation of any single BET bromodomain.

Based on the success of compounds 7 and 11-13, particularly compound 11, the inventors went on to synthesise further molecules for testing (compounds AL, ME-Am₁, ET-Am₁, AL-Am₁, ME-Am₂, ET-Am₂, 9-ME, 9-ET, 9-AL, 9-ME-Am₁ and 9-ET-Am₁).

Thermal stabilization for wild type constructs of Brd2(1) and (2) as well as their respective leucine to alanine, valine and isoleucine mutants in the presence of the new compounds in addition to compounds 7, 11 and 12 and negative controls I-BET, 9-I-BET, I-BET-OMe and 9-I-BET-OMe are shown in FIG. 15.

To investigate the utility of the new compounds further, the inventors performed isothermal titration experiments by titrating 250 μM compound solutions (AL, ME-Am₁, ET-Am₁, ME-Am₂, ET-Am₂, 9-ME, 9-ET, 9-ME-Am₁ and 9-ET-Am₁) 25 μM protein solutions (WT and mutants (Leu to Ala, Leu to Val and Leu to Ile) of Brd2(2)). The results obtained by ITC mirror what was seen in the DSF analysis, showing higher selectivity for the mutants relative to wild types, the effect being slightly less pronounced in the leucine to isoleucine mutants.

As expected, compounds I-BET, 9-I-BET, 9-I-BET-OMe and I-BET-OMe showed no selective stabilization of the mutant proteins. As shown previously, compounds 7 and 11 showed increased thermal shifts of the leucine to alanine mutants. Such increased thermal shifts were also seen for the leucine to valine mutants and the Brd2(2) leucine to isoleucine mutant. However, the Brd2(1) leucine to isoleucine mutant showed little stabilisation. Similar results were observed for the AL, ME-Am₁, ET-Am₁, ME-Am₂, ET-Am₂, 9-ME, 9-ET, 9-ME-Am₁ with smaller shifts observed for PR and 9-ET-Am₁.

The inventors anticipate that further optimisation of iBET analogues will yield additional chemical probes with still higher selectivity.

### Materials and Methods

### Plasmids and Peptides

Plasmids of the eight single BET bromodomain constructs Brd2(1), Brd2(2), Brd3(1), Brd3(2), Brd4(1), Brd4(2), Brdt(1) and Brdt(2) were provided by the Structural Genomics Consortium (SGC) at the University of Oxford in the United Kingdom. Constructs contain a His₆-tag on the N-terminus of the protein. A plasmid of pEGFP-C1 containing the whole Brd4 gene was also provided by the SGC for fluorescence recovery after photobleaching experiments. The plasmid for the full length Brd2 protein was purchased from DNASU Plasmid Repository at the Arizona State University and a tandem construct containing a His₆-tag, a Small Ubiquitin-like Modifier (SUMO) tag, as well as both bromodomains and the linker domain was cloned.

A tetra acetylated peptide mimicking the acetylated histone tail H4 (KAc 5, 8, 12, 16), identified as a natural binding partner of BET bromodomains with the sequence SEQ ID 1: YSGRGK(Ac)GGK(Ac)GLGK(Ac)GGAK(Ac)RHRK was synthesized and purified by GenScript.

### Site Directed Mutagenesis

Single point mutations were introduced using QuickChange II Site directed Mutagenesis Kit from Agilent. Primers were designed following the recommendations in the QuickChange Manual and oligonucleotides were synthesized, desalted, purified and lyophilized by Sigma Aldrich. The polymerase chain reaction was performed on a 2720 Thermal Cycler from Applied Biosystems®. Upon digestion of the parental DNA strands, the PCR product was transformed and grown on LB agar plates containing 50 μg/ml of kanamycin at 37° C. for 12-16 hours. Single colonies were then picked from the agar plates and grown for 12 hours in 10 ml of LB medium and 50 μg/ml of Kanamycin. DNA was subsequently extracted and purified using QIAprep Spin Miniprep Kit from Qiagen. Purified DNA was then submitted to sequencing to confirm the presence of the desired mutations.

### Protein Expression

Single colonies from freshly transformed plasmid DNA in competent E. coli BL21(DE3) cells were grown overnight at 37° C. in 10 ml of LB medium with 50 μg/ml kanamycin. The start-up culture was then diluted 1:100 in fresh Terrific-Broth medium with 50 μg/ml of kanamycin and 4 ml of glycerol. Cell growth was allowed at 37° C. and 200 rpm to an optical density of about 2.5 (OD600), at which point temperature was decreased to 18° C. Once the cultures equilibrated at 18° C., the optical density was around 3.0 (OD600) and protein expression was induced overnight at 18° C. with 0.1 mM isopropyl-p-thiogalactopyranoside (IPTG). The bacteria was harvested the next day by centrifugation (8000 rpm for 10 minutes at 6° C., JLA 8.1000 rotor on a Beckman Coulter Avanti J-20 XP centrifuge) and frozen at −20° C. as pellets for storage.

### Protein Purification

Pellets of cells which express His₆-tagged proteins were resuspended in lysis buffer (50 mM HEPES pH 7.5 at 25° C., 500 mM NaCl, 10 mM Imidazole and 2 mM β-mercaptoethanol). One tablet of Complete Protease Inhibitor Cocktail from Roche was added to the resuspension and cells were lysed using a French Press at 4° C. Following a 20 min incubation period at room temperature with 10 μg/ml DNaseI and 10 mM MgCl₂, the cell debris was removed by centrifugation (20000 rpm for 30 minutes at 6° C., JA25.50 rotor in a Beckman Coulter Avanti J-20XP centrifuge). The lysate was purified via immobilized metal ion affinity chromatography on a His Trap HP 5 ml Ni sepharose column (GE Healthcare Life Sciences) on an ÄKTAexplorer™ system (GE Healthcare) or an ÄKTApure™ system (GE Healthcare). The column was equilibrated by 25 ml of lysis buffer and the flow was set to 1 ml/min. His₆ tagged protein was eluted using a linear gradient to 250 mM imidazole in the same buffer. In some cases, the His₆ tag was removed after this by overnight treatment with Tobacco Etch Virus (TEV) protease at 4° C. followed by a second Ni column to collect the flow through. The same procedure was followed to cleave the SUMO tag from tandem constructs using sentrin-specific protease 1 (SENP1) instead of TEV. After Ni purification, the pooled elution fractions were concentrated to a volume of 4 ml and further purified by size exclusion chromatography on a Superdex 75 16/60 Hiload gel filtration column (GE Healthcare) on an ÄKTAexplorer™ or an ÄKTApure™ system using the following buffer: 10 mM HEPES pH 7.5 at 25° C., 500 mM NaCl and 5% glycerol. Samples were monitored by SDS-polyacrylamide gel electrophoresis to verify purity. Pure protein was then flash frozen with liquid nitrogen and stored at −80° C. The mass and purity of the proteins were subsequently verified by mass spectrometry (MALDI-TOF).

### Differential Scanning Fluorimetry

DSF assays were performed on a LightCycler®480 from Roche or a Mx3005P Real Time PCR machine from Stratagene. Prior to DSF assays, frozen proteins were buffer exchanged using Vivaspin®6 concentrators with a 10 kDa cutoff on a Centrifuge 5430 from Eppendorf at a speed of 6000×g to remove glycerol and to buffer the proteins in 20 mM HEPES pH 7.5 at 25° C. and 100 mM NaCl. SYPRO®Orange from Invitrogen Molecular Probes® was used as a reporter dye to monitor the denaturing process of the proteins. Samples were assayed on a 96-well plate with final protein concentrations of 2 μM for the LightCycler®480 and 6 μM for Mx3005P. Compounds were added at a final concentration of 10 μM for the LightCycler®480 and 30 μM for Mx3005P, while the tetra acetylated histone peptide was added to a final concentration of 100 μM and 300 μM respectively. SYPRO®Orange was added at a dilution of 1:1000 and excitation and emission filters for the SYPRO®Orange dye were set to 483 nm and 568 nm respectively for the LightCycler®480 and 465 nm and 590 nm respectively for Mx3005P. The temperature was raised with a step of 0.6° C. per minute from 37° C. to 95° C. with the LightCycler®480 collecting 39 measurements per ° C., and 1° C. per minute from 25° C. to 95° C. with Mx3005P, collecting fluorescence readings at the end of each interval. Each sample was run in triplicates.

Collected data was analysed by IGOR Pro 6, a scientific software tool from Wave Metrics, Inc. Analysis was done following the recommendations of Niesen et al. [11]. Fluorescence intensity was plotted as a function of temperature, generating a sigmoidal curve described by a two-state transition from folded to unfolded protein. Curves were fitted by the following sigmoidal equation:

$$f ( x ) = {A}_{1} + \frac{{A}_{2}}{1 + {exp}^{\frac{{x}_{0} - x}{dx}}}$$

A₁ and A₂ are the values of minimum and maximum intensities, respectively, x₀ is the inflection point and dx is the rate. Fitted curves were differentiated in IGOR Pro 6 and the maximum of the first derivative was identified using the same program. These values correspond to the inflection points of the transition curves and thus to the melting temperatures of the proteins (Tₘ).

### Isothermal Titration Calorimetry

ITC experiments were carried out on a ITC200 instrument from MicroCal™ Experiments were conducted at 3 different temperatures 15° C., 25° C. and 30° C., while stirring at 1000 rpm. Buffers of proteins, peptide and compounds were matched to 20 mM HEPES pH 7.5 at 25° C. and 100 mM NaCl. Frozen protein was buffer exchanged as described for the DSF experiments. Each titration comprised 1 initial injection of 0.4 μl lasting 0.8 s, followed by 19 injections of 2 μl lasting 4 s each at 2 min intervals. The initial injection was discarded during data analysis. Standard and reverse titrations were conducted depending on the binding partners.

### Peptide Binding

Experiments with the tetra acetylated histone peptide were performed at 15° C. The micro syringe (40 μl) was loaded with a solution of the peptide sample at a concentration of 1-2 mM and it was injected into the cell (200 μl), occupied by a protein at a concentration of 50-100 μM.

### Ligand Binding

Reverse titrations were conducted to test the binding of the known ligands and the novel chemical probes to the wild types and the mutants. Experiments were carried out either at 25° C. or 30° C. For strong binders, a concentration of 150-200 μM of the protein was injected into a solution of 15-20 μM compound. For lower affinity interactions, a concentration of 350 μM protein was titrated into a solution of 20 μM compound. In cases where the compound was solubilized in dimethyl sulfoxide, DMSO concentration was adjusted to 1% both in the syringe and in the cell.

### Data Analysis

All the data was fitted to a single binding site model using the Microcal LLC ITC200 Origin software provided by the manufacturer to yield enthalpies of binding (ΔH) and binding constants (Kₐs). Further thermodynamic parameters were calculated from these values (changes in entropy ΔS, changes in free energy AG and dissociation constants (K_{d}s)).

### Docking:

Mutant models (V/A, L/A, W/F) were obtained by introducing specific mutations with the Maestro editing tools, using the crystal structure of brd4(1) (pdb 3P5O(2)) as a template. WT and mutant 3P5O were prepared using the Protein Preparation Wizard(3) from Schrodinger, and the corresponding grids were generated with Glide. (4), (5), (6), (7) Ligands were prepared (Ligprep(8)) and docked (Glide) in mutant and VVT grids. No constraint was applied to the system. Docking poses were subjected to one round of Prime(9) minimisation, then analysed visually with Maestro and Pymol (10).

### Synthesis:

All reagents and solvents were obtained from commercial sources, and used as supplied unless otherwise indicated. Reactions requiring anhydrous conditions were conducted in heated glassware (heat gun), under an inert atmosphere (argon), and using anhydrous solvents. CH₂Cl₂ and MeOH were distilled over CaH₂. THF and Et₂O were distilled on Na/benzophenone. Toluene was distilled over Na. All reactions were monitored by analytical thin-layer chromatography (TLC) using indicated solvent systems on E. Merck silica gel 60 F254 plates (0.25 mm). TLC plates were visualized using UV light (254 nm) and/or by staining in potassium permanganate followed by heating. Solvents were removed by rotary evaporator below 40° C. and the compounds further dried using high vacuum pumps.

¹H and ¹³C NMR were recorded on a Bruker Advance 400 spectrophotometer at 400 MHz and 100 MHz respectively. Chemical shifts (δ H) are quoted in ppm (parts per million) and referenced to residual solvent signals: ¹H δ=7.26 (CDCl₃), 2.50 (d6-DMSO), 3.31 (CD₃OD), ¹³C δ=77.0 (CDCl₃), 39.43 (d₆-DMSO), 49.05 (CD₃OD). Coupling constants (J) are given in Hz. High resolution mass spectra (ESI) were recorded on a Waters LCT Premier Mass Spectrometer.

Purification by preparative HPLC was performed on a Varian Prostar; column: Pursuit C18, 5 μm, 250×21.2 mm; solvent: gradient 0:100 to 100:0 MeCN/H₂O over 30 minutes, 0.1% TFA (constant), flow rate 12 ml/min.

### Intermediate 19 (N-(2-(4-chlorobenzoyl)-4-methoxy-6-methylphenyl)acetamide)

To a suspension of N-(4-methoxy-2-methylphenyl)acetamide 17 (6.20 g, 34.6 mmol, 1.0 eq.) in freshly distilled toluene (70 mL) were added Pd(TFA)₃ (1.15 g, 3.46 mmol, 0.10 eq.), 4-chlorobenzaldehyde 18 (12.2 g, 86.5 mmol, 2.5 eq.) and tert-butylhydroperoxide (70% aq., 19.2 mL, 138 mmol, 4.0 eq.). The resulting mixture was stirred at reflux for 24 h, then cooled to rt. Saturated aqueous NaHCO₃ (300 mL) was added. The aqueous phase was extracted with EtOAc (3×300 mL) and CHCl₃ (1×300 mL). The combined organic phases were dried (MgSO₄) and concentrated. The product (3.35 g, 30%) was obtained after purification by flash column chromatography (gradient hexane/EtOAc 1:1 to 2:8). Rf 0.2 (hexane/AcOEt 1:1); ¹H NMR (400 MHz, CDCl₃) δ 2.00 (s, 3H); 2.28 (s, 3H), 3.76 (s, 3H), 6.71 (d, J=2.8 Hz, 1H), 6.94 (d, J=2.8 Hz, 1H), 7.43 (m, 2H), 7.78 (m, 2H), 7.87 (s, 1H); ¹³C NMR (100 MHz, CDCl₃) δ 18.9, 23.4, 55.6, 113.1, 118.8, 127.2, 128.7, 131.8, 135.2, 135.4, 138.1, 139.8, 157.0, 168.8, 195.9; HRMS (ESI+) m/z calc. for C₁₇H₁₇ClNO₃ [M+H]⁺ 318.0891. found: 318.1255.

### Intermediate 20 ((2-amino-5-methoxy-3-methylphenyl)(4-chlorophenyl)methanone)

To a solution of acetyl protected aminobenzophenone 19 (1.00 g, 3.15 mmol, 1.0 eq.) in iPrOH (10 mL) was added 36% aq. HCl (5 mL). The resulting mixture was heated for 2 hours at 130° C. under microwave irradiation. After cooling to rt, the pH was adjusted to 7-9, and the aqueous phase was extracted with CH₂Cl₂ (3×50 mL). The combined organic phases were dried (MgSO₄) and concentrated. The product (657 mg, 76%) was obtained as a yellow solid after purification by flash column chromatography (gradient hexane/AcOEt 85:15 to 30:70). Rf 0.8 (hexane/AcOEt 1:1). ¹H NMR (400 MHz, CDCl₃) δ 2.22 (s, 3H), 3.64 (s, 3H), 5.81 (br. s, 2H), 6.78 (d, J=2.8 Hz, 1H), 6.94 (d, J=2.8 Hz, 1H), 7.43 (m, 2H), 7.61 (m, 2H); ¹³C NMR (100 MHz, CDCl₃) δ 17.6, 55.9, 114.5, 117.4, 124.1, 125.4, 128.4, 130.6, 137.4, 138.5, 144.1, 149.1, 197.5; HRMS (ESI+) m/z calc. for C₁₅H₁₅ClNO₂ [M+H]⁺ 276.0786. found: 276.0897.

### Intermediate 22 (Methyl-2-(5-(4-chlorophenyl)-7-methoxy-9-methyl-2-oxo-2,3-dihydro-1H-benzo[e][1,4]diazepin-3-yl)acetate)

A solution of 21 (2.30 g, 6.35 mmol, 1 eq.) and thionyl chloride (4.61 mL, 63.5 mmol, 10 eq.) in freshly distilled CH₂Cl₂ (35 mL), under inert atmosphere (argon), was refluxed for 2.5 hours. After cooling to rt, the volatiles were removed in vacuo. The residue was dissolved in CHCl₃ (30 mL) under inert atmosphere (argon), and benzophenone 20 (1.75 g, 6.35 mmol, 1 eq.) was added. The resulting mixture was refluxed for 3 hours, then cooled to rt. Et₃N (3.54 mL, 25.4 mmol, 4 eq.) was added and the mixture was refluxed for an additional 16 hours, cooled to rt and concentrated to dryness. The residue was dissolved in 1,2-dichloroethane (80 mL) and AcOH (4.0 mL), stirred at 60° C. for 3 hours, cooled to rt, and finally concentrated in vacuo. The residue was diluted with saturated aqueous NaHCO₃ (80 mL) and the aqueous phase was extracted with CH₂Cl₂ (3×100 mL). The combined organic phases were dried (MgSO₄) and concentrated in vacuo. The product (2.28 g, 93%) was obtained as a white amorphous solid after flash column chromatography (6:4 hexane/AcOEt). Rf 0.45 (hexane/AcOEt 1:1). ¹H NMR (400 MHz, CDCl₃) δ 2.42 (s, 3H), 3.17 (dd, J=16.8, 6.9 Hz, 1H), 3.39 (dd, J=16.8, 7.4 Hz, 1H), 3.70 (s, 3H), 3.73 (s, 3H), 4.12 (app-t, J=7.0 Hz, 1H), 6.57 (d, J=2.9 Hz, 1H), 6.97 (d, J=2.9 Hz, 1H), 7.31 (m, 2H), 7.48 (m, 2H), 8.84 (s, 1H). ¹³C NMR (100 MHz, CDCl₃) δ 18.4, 36.2, 51.7, 55.6, 60.2, 111.8, 120.4, 128.4, 128.6, 130.4, 131.1, 131.6, 136.5, 137.4, 154.9, 168.4, 170.5, 172.4. HRMS (ESI+) m/z calc. for C₂₀H₂₀ClN₂O₄ [M+H]⁺ 387.1106. found: 387.1276.

### Intermediate 23 (Methyl 2-(5-(4-chlorophenyl)-7-methoxy-9-methyl-2-thioxo-2,3-dihydro-1H-benzo[e][1,4]diazepin-3-yl)acetate)

A solution of amide 22 (2.10 g, 5.43 mmol, 1 eq.) and Lawesson's reagent (1.32 g, 3.26 mmol, 0.6 eq.) in freshly distilled toluene (36 mL) was refluxed under inert atmosphere (argon) for 5 hours. After cooling to rt, the reaction mixture was concentrated to dryness. The product (1.92 g, 88%) was obtained as a light yellow amorphous solid after flash column chromatography (gradient 98:2 to 95:5 CHCl₃/AcOEt). Rf 0.5 (hexane/AcOEt 7:3); ¹H NMR (400 MHz, CDCl₃) δ 2.42 (s, 3H), 3.39 (dd, J=16.8, 7.2 Hz, 1H), 3.62 (dd, J=16.8, 6.6 Hz, 1H), 3.72 (s, 3H), 3.73 (s, 3H), 4.36 (app-t, J=6.8 Hz, 1H), 6.62 (d, J=2.8 Hz, 1H), 6.98 (d, J=2.8 Hz, 1H), 7.34 (m, 2H), 7.49 (m, 2H), 9.19 (s, 1H); ¹³C NMR (100 MHz, CDCl₃) δ 18.3, 39.6, 51.7, 55.6, 63.7, 112.3, 120.3, 128.4, 130.0, 130.8, 131.1 (2C), 136.7, 137.0, 155.9, 167.8, 172.3, 200.5; HRMS (ESI+) m/z calc. for C₂₀H₂₀ClN₂O₃S [M+H]⁺ 403.0878. found: 403.1353.

### Compound 4 Methyl 2-(6-(4-chlorophenyl)-8-methoxy-1,10-dimethyl-4H benzo[f][1,2,4]triazolo[4,3-a][1,4]diazepin-4-yl)acetate

To an ice cold solution of thioamide 23 (1.70 g, 4.22 mmol, 1 eq.) in freshly distilled THF (55 mL), under inert atmosphere (argon), was added hydrazine monohydrate (614 μL, 12.7 mmol, 3 eq.) dropwise. The resulting mixture was stirred at 0° C. for 5.5 hours. Et₃N (1.76 mL, 12.7 mmol, 3 eq.) and acetyl chloride (900 μL, 12.7 mmol, 3 eq.) were added dropwise. After stirring for a few minutes at 0° C. and 16 hours at rt, the volatiles were removed in vacuo. The residue was dissolved in CH₂Cl₂ (100 mL) and washed with water (70 mL). The organic phase was dried (MgSO₄) and concentrated in vacuo.

The residue was dissolved in freshly distilled THF (18 mL) under an inert atmosphere (argon) and AcOH (11 mL) was added. The resulting mixture was stirred at 100° C. for 3 hours. The volatiles were removed in vacuo and the product (676 mg, 38%) was obtained as an amorphous white solid after flash column chromatography (gradient 97:3 to 95:5 CH₂Cl₂/MeOH). Rf 0.4 (CH₂Cl₂/MeOH 96:4); ¹H NMR (400 MHz, CDCl₃) δ 2.30 (s, 3H), 2.44 (s, 3H), 3.55 (dd, J=17.0, 6.1 Hz, 1H), 3.63 (dd, J=17.0, 8.4 Hz, 1H), 3.75 (s, 3H), 3.79 (s, 3H), 4.56 (m, 1H), 6.69 (d, J=2.7 Hz, 1H), 7.04 (d, J=2.7 Hz, 1H), 2.53 (m, 2H), 7.52 (m, 2H); ¹³C NMR (100 MHz, CDCl₃) δ 11.8, 18.9, 36.3, 51.9, 53.1, 55.7, 113.4, 119.2, 125.1, 128.4, 130.6, 131.7, 135.0, 136.9 (2C), 152.2, 157.1, 158.3, 166.0, 172.1; HRMS (ESI+) m/z calc. for C₂₂H₂₂ClN₄O₃ [M+H]⁺ 425.1375. found: 425.1913.

### Alkylation

A −78° C. solution of 24 (16) (400 mg, 0.973 mmol, 1.0 eq.) in freshly distilled THF (6 mL), under Ar, was added dropwise by canulation to a −78° C. solution of KHMDS (0.5M in toluene, 2.34 mL, 1.17 mmol, 1.2 eq.) in freshly distilled THF (14 mL), under Ar. The resulting dark solution was stirred at −78° C. for 1 h. MeI (73 μL, 1.17 mmol, 1.2 eq.) was then added dropwise, and stirring was continued for 1 h at −78° C. The temperature of the acetone bath was then gradually increased to rt over a few hours, and the mixture was stirred overnight at rt. The reaction was quenched with a few drops of AcOH and concentrated to dryness. The residue was partitioned between saturated aqueous NaHCO₃ and CHCl₃ and the aqueous phase was extracted 3 times. The combined organic layers were dried (MgSO₄) and concentrated. Purification by flash column chromatography (PE₄₀₋₆₀/acetone 6:4) afforded a mixture of (+−)-6 and (+−)-7 (232 mg, 56%) and (+−)-5 (14 mg, 3%).

### Compound 6 (+−)-methyl (S)-2-((S)-6-(4-chlorophenyl)-8-methoxy-1-methyl-4H-benzo[f][1,2,4]triazolo[4,3-a][1,4]diazepin-4-yl)propanoate

(+−)-6 was the major product of the alkylation reaction and migrated faster than (+−)-7 on silica (PE40-60/acetone).

Diastereomerically pure samples of (+−)-6 were obtained after purification by flash column chromatography of the mixture described above. Rf 0.15 (PE40-60/acetone 6:4); ¹H NMR (400 MHz, CDCl₃) δ 1.60 (d, J=7.2 Hz, 3H), 2.61 (s, 3H), 3.72 (s, 3H), 3.80-3.93 (m, 4H), 4.29 (d, J=10 Hz, 1H), 6.90 (d, J=2.9 Hz, 1H), 7.21 (dd, J=8.8, 2.9 Hz, 1H), 7.34 (m, 2H), 7.41 (d, J=8.8 Hz, 1H), 7.53 (m, 2H); ¹³C NMR (100 MHz, CDCl₃) δ 12.1, 15.4, 41.0, 52.0, 55.9, 57.7, 115.9, 117.7, 125.0, 126.5, 128.5, 130.0, 130.7, 137.0, 137.1, 150.2, 156.0, 158.0, 166.2, 175.9; HRMS (ESI+) m/z calc. for C₂₂H₂₂ClN₄O₃ [M+H]⁺ 425.1375. found: 425.1951.

### Compound 7 (+−)-methyl (R)-2-((S)-6-(4-chlorophenyl)-8-methoxy-1-methyl-4H-benzo[f][1,2,4]triazolo[4,3-a][1,4]diazepin-4-yl)propanoate

(+−)-7 was the minor product of the alkylation reaction and migrated slower than (+−)-6 on silica (PE40-60/acetone).

To a diastereomeric mixture of (+−)-6 and (+−)-7 (50 mg, 0.118 mol, 1 eq.) in anhydrous MeOH (15 mL) was added MeONa (64 mg, 1.18 mmol, 10 eq.). The resulting solution was heated at 120° C. for 40 minutes under microwave irradiation. The reaction mixture was cooled to 60° C. and a few drops of AcOH were added to quench MeONa, followed by cooling to rt and concentration in vacuo. The residue was dissolved in sat. aq. NaHCO₃ and extracted 4 times with CHCl₃. The combined organic layers were dried (MgSO₄) and concentrated in vacuo. Purification by preparative TLC (PE₄₀₋₆₀/acetone 1:1) afforded diastereomerically pure samples of (+−)-7. Rf 0.15 (PE₄₀₋₆₀/acetone 6:4); ¹H NMR (400 MHz, CDCl₃) δ 1.49 (d, J=7.0 Hz, 3H), 2.65 (s, 3H), 3.81 (s, 3H), 3.82 (s, 3H), 4.04 (m, 1H), 4.26 (d, J=10.7 Hz, 1H), 6.89 (d, J=2.8 Hz, 1H), 7.23 (dd, J=8.9, 2.8 Hz, 1H), 7.32 (m, 2H), 7.40-7.48 (m, 3H). ¹³C NMR (100 MHz, CDCl₃) δ 12.0, 15.2, 42.3, 51.9, 55.9, 59.5, 115.9, 117.9, 125.0, 126.0, 128.5, 130.0, 130.8, 136.8, 137.0, 150.5, 155.0, 158.2, 165.5, 175.9; HRMS (ESI+) m/z calc. for C₂₂H₂₂ClN₄O₃ [M+H]⁺ 425.1375. found: 425.1902.

### Compound 5 (+−)-methyl 2-(6-(4-chlorophenyl)-8-methoxy-1,4-dimethyl-4H-benzo[f][1,2,4]triazolo[4,3-a][1,4]diazepin-4-yl)acetate

Rf 0.15 (PE₄₀₋₆₀/acetone 6:4); NMR at rt revealed the presence of two conformers in solution for 5, in an approximately 65:35 ratio. For clarity only chemical shifts for the major conformer are reported below. ¹H NMR (400 MHz, CDCl₃) δ 1.36 (s, 3H), 2.64 (s, 3H), 3.40 (d, J=16 Hz, 1H), 3.70 (d, J=16 Hz, 1H), 3.78 (s, 3H), 3.80 (s, 3H), 6.84 (d, J=2.9 Hz, 1H), 7.19 (dd, J=7.2, 2.9 Hz, 1H), 7.35 (m, 2H), 7.37 (d, J=7.2 Hz, 1H), 7.51 (m, 2H); ¹³C NMR (100 MHz, CDCl3) δ 12.4, 18.4, 45.7, 51.5, 55.8, 58.0, 116.1, 117.2, 124.7, 126.7, 128.4, 130.8, 136.7, 138.2, 151.0, 158.0, 158.3, 163.6, 171.6; HRMS (ESI+) m/z calc. for C₂₂H₂₂ClN₄O₃ [M+H]⁺ 425.1375. found: 425.1974.

### Intermediate 26 ((+−)-Methyl 2-(7-methoxy-2,5-dioxo-2,3,4,5-tetrahydro-1H-benzo[e][1,4]diazepin-3-yl)acetate)

Isatoic anhydride derivative 25 (3.70 g, 19.2 mmol, 1 eq.) and aspartic acid dimethylester (3.77 g, 19.2 mmol, 1 eq.) were suspended in pyridine at rt, under an inert atmosphere (argon). The temperature was gradually increased until reflux was reached. Reflux was continued for 24 hours. After cooling to rt, the reaction mixture was concentrated to dryness. The residue was triturated in a ˜94:6 CH₂Cl₂/MeOH mixture and a first crop (1.27 g) of the product could be obtained as a white solid after filtration and washing with small amounts of CH₂Cl₂. The filtrate was concentrated to dryness and submitted to flash column chromatography (96:4 CH₂Cl₂/MeOH) and the fractions containing the impure product were concentrated in vacuo. The residue was triturated in a ˜94:6 CH₂Cl₂/MeOH mixture and a second crop (504 mg) of the product could be obtained as a white solid after filtration and washing with small amounts of CH₂Cl₂. Total: 1.77 g, 36%. Rf 0.45 (AcOEt); ¹H NMR (400 MHz, d6-DMSO) δ 2.72 (dd, J=17.1, 6.1 Hz, 1H), 2.88 (d, J=17.1, 8.3 Hz, 1H), 3.58 (s, 3H), 3.79 (s, 3H), 3.98-4.05 (m, 1H), 7.06 (d, J=9.0 Hz, 1H), 7.16 (dd, J=9.0, 3.1 Hz, 1H), 7.22 (d, J=3.1 Hz, 1H), 8.59 (s, 1H), 10.3 (s, 1H); ¹³C NMR (100 MHz, d6-DMSO) δ 32.4, 48.5, 51.6, 55.5, 113.3, 119.5, 122.8, 127.2, 129.9, 155.7, 167.3, 170.4, 170.6; HRMS (ESI+) m/z calc. for C₁₃H₁₅N₂O₅[M+H]⁺ 279.0975. found: 279.1001.

### Intermediate 27 ((+−)-Methyl 2-(7-methoxy-5-oxo-2-thioxo-2,3,4,5-tetrahydro-1H-benzo[e][1,4]diazepin-3-yl)acetate)

To a suspension of diamide 26 (1.86 g, 6.68 mmol, 1 eq.) in pyridine at rt, under inert atmosphere (argon), was added Lawesson's reagent (1.62 g, 4.01 mmol, 0.6 eq.). The resulting mixture was heated at reflux for 1.25 hour. After cooling to rt, the volatiles were removed in vacuo. The residue was suspended in CH₂Cl₂ and a first crop (930 mg) of product was obtained as a light yellow powder after filtration and washing with small amounts of CH₂Cl₂. The filtrate was concentrated to dryness and submitted to flash column chromatography (gradient 8:2 to 1:1 CH₂Cl₂/AcOEt) and the fractions containing the impure product were concentrated in vacuo. The residue was triturated in CH₂Cl₂ and a second crop (190 mg) of the product could be obtained as a light yellow powder after filtration and washing with small amounts of CH₂Cl₂. Total: 1.12 g, 57%. Rf 0.3 (PE₄₀₋₆₀/AcOEt 1:1); ¹H NMR (400 MHz, d6-DMSO) δ 2.83 (dd, J=17.1, 6.2 Hz, 1H), 3.22 (dd, J=17.1, 7.6 Hz, 1H), 3.57 (s, 3H), 3.82 (s, 3H), 4.22 (m, 1H), 7.17-7.28 (m, 3H), 8.82 (d, J=5.9 Hz, 1H), 12.3 (s, 1H); ¹³C NMR (100 MHz, d6-DMSO) δ 35.8, 39.6, 51.5, 55.6, 113.4, 119.3, 123.3, 128.7, 130.0, 156.9, 166.5, 170.4, 200.6; HRMS (ESI+) m/z calc. for C₁₃H₁₅N₂O₄S [M+H]⁺ 295.0747. found: 295.0831.

### Intermediate 28 ((+−)-methyl 2-(8-methoxy-1-methyl-6-oxo-5,6-dihydro-4H-benzo[f][1,2,4]triazolo[4,3-a][1,4]diazepin-4-yl)acetate)

To a suspension of thioamide 27 (2.20 g, 7.48 mmol, 1 eq.) in freshly distilled THF (33 mL) were successively added AcOH (22 mL) and acethydrazide (1.66 g, 22.4 mmol, 3 eq.). The reaction mixture was cooled to 0° C. and mercury (II) acetate (3.58 g, 11.2 mmol, 1.5 eq.) was added. The mixture was stirred for 2 hours at 0° C., and for a further 3 days at rt. After filtration on celite, the volatiles were removed in vacuo, and the product (2.15 g, 91%) was obtained as a white amorphous solid after flash column chromatography (95:5 CH₂Cl₂/MeOH). Rf 0.4 (CH₂Cl₂/MeOH 9:1); ¹H NMR (400 MHz, CDCl₃) δ 2.57 (s, 3H), 3.20 (dd, J=16.8, 7.3 Hz, 1H), 3.54 (dd, J=16.8, 6.5 Hz, 1H), 3.73 (s, 3H), 3.93 (s, 3H), 4.78 (m, 1H), 7.20 (dd, J=8.8, 2.8 Hz, 1H), 7.27 (d, J=8.8 Hz, 1H), 7.51 (d, J=2.8 Hz, 1H), 7.94 (br. d, J=4.9 Hz, 1H); ¹³C NMR (100 MHz, CDCl₃) δ 12.0, 33.3, 44.0, 52.3, 55.9, 115.3, 119.4, 123.7, 124.6, 130.1, 151.3, 154.6, 159.3, 167.9, 170.2; HRMS (ESI+) m/z calc. for C₁₅H₁₇N₄O₄ [M+H]⁺ 317.1244. found: 317.1289.

### Intermediate 29 ((+−)-methyl 2-(6-chloro-8-methoxy-1-methyl-4H-benzo[f][1,2,4]triazolo[4,3-a][1,4]diazepin-4-yl)acetate)

To amide 28 (170 mg, 0.537 mmol, 1 eq.) in CHCl₃ (14 mL), at rt and under an inert atmosphere (argon), were successively added N,N-dimethylaniline (375 μL, 2.96 mmol, 5.5 eq.) and POCl₃ (1.05 mL, 11.3 mmol, 21 eq.). The resulting mixture was stirred 125° C. (sealed tube) for 1 hour, then cooled to 0° C. Et₃N (1.35 mL) was added dropwise. The volatiles were removed in vacuo. This procedure was repeated on twelve batches (total: 2.04 g). The twelve batches were then combined and the product (631 mg, 29%) was obtained as a white amorphous solid after flash column chromatography (3:7 CH₂Cl₂/acetone). Of note, attempted purification with MeOH containing mixtures lead to decomposition of the imidoyl chloride. Attempted subsequent palladium mediated coupling of aryl boronic acids with crude imidoyl chloride 29 lead to poor conversion and mainly degradation. Rf 0.5 (CH₂Cl₂/acetone 3:7); ¹H NMR (400 MHz, CDCl₃) δ 2.63 (s, 3H), 3.47 (dd, J=17.3, 8.3 Hz, 1H), 3.57 (dd, J=17.3, 6.0 Hz, 1H), 3.73 (s, 3H), 3.94 (s, 3H), 4.66 (m, 1H), 7.24 (dd, J=9.0, 2.8 Hz, 1H), 7.39 (d, J=9.0 Hz, 1H), 7.45 (d, J=2.8 Hz, 1H);); ¹³C NMR (100 MHz, CDCl₃) δ 12.2, 36.2, 52.1, 53.4, 56.0, 114.9, 119.4, 124.2, 124.8, 129.4, 151.2, 154.0, 154.4, 158.9, 171.3; HRMS (ESI+) m/z calc. for C₁₅H₁₆C1N₄O₃ [M+H]⁺ 335.0905. found: 335.0950.

General Procedure for the Coupling of Imidoyl Chloride with Phenylboronic Acid Derivatives:

To a suspension imidoyl chloride derivative 29 (30 mg, 0.0896 mmol, 1 eq.), arylboronic acid (0.116 mmol, 1.3 eq.), Pd(PPh₃)₄ (15.5 mg, 0.0134 mmol, 0.15 eq.) in anhydrous DMF (1 mL) and under an argon atmosphere was added Et₃N (50 μL, 0.358 mmol, 4 eq.) at rt. The vessel was sealed and the mixture was stirred at 100° C. for 24 h. After cooling to rt, DMF was evaporated in vacuo. The product was purified by flash column chromatography and further purified by reverse phase preparative HPLC when necessary.

### Compound 8 (+−)-methyl 2-(6-(4-chloro-2-methylphenyl)-8-methoxy-1-methyl-4H-benzo[f][1,2,4]triazolo[4,3-a][1,4]diazepin-4-yl)acetate

8 was prepared according to the general procedure described above, and was obtained as a light yellow solid after purification by flash column chromatography (CH₂Cl₂/MeOH 95:5); Rf 0.3 (CH₂Cl₂/MeOH 95:5); ¹H NMR (400 MHz, CDCl₃) δ 2.37 (s, 3H), 2.68 (s, 3H), 3.55-3.68 (m, 2H), 3.77 (s, 3H), 3.81 (s, 3H), 4.60 (app-t, J=7.0 Hz, 1H), 6.89 (d, J=2.8 Hz, 1H), 7.22 (d, J=8.7, 2.8 Hz, 1H), 7.20-7.24 (m, 2H), 7.31 (d, J=8.4 Hz, 1H), 7.43 (d, J=8.8 Hz, 1H), 7.46 (d, J=1.7 Hz, 1H); ¹³C NMR (100 MHz, CDCl₃) δ 12.1, 20.1, 36.6, 51.9, 53.0, 55.9, 116.1, 117.9, 124.9, 126.0, 128.3, 128.9, 130.3, 131.6, 136.3, 137.0, 137.2, 150.6, 156.0, 158.2, 166.6, 172.0; HRMS (ESI+) m/z calc. for C₂₂H₂₂ClN₄O₃ [M+H]⁺ 425.1375. found: 425.1419.

### Compound 9 (+−)-methyl 2-(6-(4-chloro-3-methylphenyl)-8-methoxy-1-methyl-4H-benzo[f][1,2,4]triazolo[4,3-a][1,4]diazepin-4-yl)acetate

9 was prepared according to the general procedure described above, and was obtained as a mono TFA salt after purification by flash column chromatography (gradient PE₄₀₋₆₀/acetone 3:7 to 2:8) and reverse phase preparative HPLC, RT=24 min. Rf 0.35 (PE₄₀₋₆₀/acetone 3:7); ¹H NMR (400 MHz, CDCl₃) δ 2.00 (s, 3H), 2.81 (s, 3H), 3.53 (dd, J=16.8, 5.2 Hz, 1H), 3.61 (dd, J=16.8, 8.9 Hz, 1H), 3.76 (s, 3H), 3.80 (s, 3H), 4.72 (m, 1H), 6.72 (d, J=2.6 Hz, 1H), 7.09-7.30 (m, 4H), 7.48 (d, J=8.6 Hz, 1H); ¹³C NMR (100 MHz, CDCl₃) δ 11.4, 20.0, 36.1, 52.1, 52.7, 56.0, 116.1, 117.6, 123.8, 125.0, 126.2, 131.1, 131.3, 132.0, 136.0, 136.7, 138.5, 150.9, 155.6, 159.4, 168.5, 171.5. HRMS (ESI+) m/z calc. for C₂₂H₂₂ClN₄O₃ [M+H]⁺ 425.1375. found: 425.1416.

### Compound 10 (+−)-methyl 2-(6-(2,5-dimethylphenyl)-8-methoxy-1-methyl-4H-benzo[f][1,2,4]triazolo[4,3-a][1,4]diazepin-4-yl)acetate

10 was prepared according to the general procedure described above, and was obtained as a white solid after purification by flash column chromatography (gradient CH₂Cl₂/MeOH 99:1 to 96:4). Rf 0.3 (CH₂Cl₂/MeOH 95:5); ¹H NMR (400 MHz, CDCl₃) δ 1.86 (s, 3H), 2.30 (s, 3H), 2.62 (s, 3H), 3.57 (dd, J=16.8, 5.3 Hz, 1H), 3.65 (dd, J=16.8, 8.7 Hz, 1H), 3.75 (s, 3H), 3.76 (s, 3H), 4.66 (m, 1H), 6.72 (d, J=2.8 Hz, 1H), 7.01 (m, 2H), 7.08 (d, J=7.7 Hz, 1H), 7.15 (dd, J=8.9, 2.8 Hz, 1H), 7.38 (d, J=8.9 Hz, 1H); ¹³C NMR (100 MHz, CDCl₃) δ 12.1, 19.2, 20.8, 36.6, 51.8, 53.1, 55.8, 115.7, 117.0, 124.4, 125.6, 129.9, 130.4, 130.8, 132.2, 132.8, 135.4, 139.0, 150.5, 156.1, 158.3, 169.5, 172.2; HRMS (ESI+) m/z calc. for C₂₃H₂₅N₄O₃ [M+H]⁺ 405.1921. found: 405.1956.

### Intermediate 24 ((+−)-methyl 2-(6-(4-chlorophenyl)-8-methoxy-1-methyl-4H-benzo[f][1,2,4]triazolo[4,3-a][1,4]diazepin-4-yl)acetate)

24 was prepared according to methods as previously described. It may also be prepared using the general procedure described above to couple imidoyl chloride 29 with 4-chlorophenylboronic acid (data not shown).

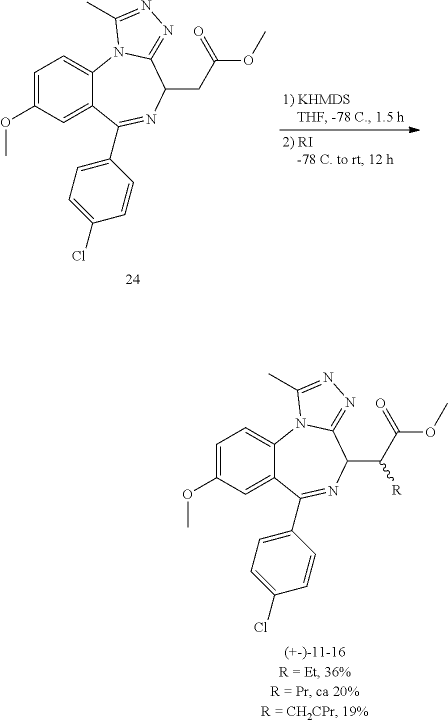

### Ethylation of the Side Chain

A −78° C. solution of 24 (400 mg, 0.973 mmol, 1.0 eq.) in freshly distilled THF (6 mL), under Ar, was added dropwise by canulation to a −78° C. solution of KHMDS (0.5 M in toluene, 2.34 mL, 1.17 mmol, 1.2 eq.) in freshly distilled THF (14 mL), under Ar. The resulting dark solution was stirred at −78° C. for 1 h. EtI (94 μL, 1.17 mmol, 1.2 eq.) was then added dropwise, and stirring was continued for 1 h at −78° C. The temperature of the acetone bath was then gradually increased to rt over a few hours, and the mixture was stirred overnight at rt. The reaction was quenched with a few drops of AcOH and concentrated to dryness. The residue was partitioned between saturated aqueous NaHCO₃ and CHCl₃ and the aqueous phase was extracted 3 times. The combined organic layers were dried (MgSO₄) and concentrated. NMR of the crude material revealed the formation of a 1/2.2/1.18 mixture of (+−)-11, (+−)-14 and (+−)-24 respectively. Purification by flash column chromatography (PE40-60/acetone 6:4) afforded a mixture of (+−)-11 and (+−)-14 (152 mg, 36%).

### Compound 11 ((+−)-methyl (R)-2-((S)-6-(4-chlorophenyl)-8-methoxy-1-methyl-4H-benzo[f][1,2,4]triazolo[4,3-a][1,4]diazepin-4-yl)butanoate)

(+−)-11 was the minor product of the alkylation reaction and migrated faster than (+−)-14 on silica (CH₂Cl₂/MeOH).

To a diastereomeric mixture of (+−)-11 and (+−)-14 (50 mg, 0.114 mol, 1 eq.) in anhydrous MeOH (15 mL) was added MeONa (62 mg, 1.14 mmol, 10 eq.). The resulting solution was heated at 120° C. for 40 minutes under microwave irradiation. The reaction mixture was cooled to 60° C. and a few drops of AcOH were added to quench MeONa, followed by cooling to rt and concentration in vacuo. The residue was dissolved in sat. aq. NaHCO₃ and extracted 4 times with CHCl₃. The combined organic layers were dried (MgSO₄) and concentrated in vacuo. Purification by flash column chromatography (gradient CH₂Cl₂/MeOH 98:2 to 96:4) afforded diastereomerically pure samples of (+−)-11. Rf 0.15 (PE₄₀₋₆₀/acetone 6:4); ¹H NMR (400 MHz, CDCl₃) δ 1.01 (t, J=7.4 Hz, 1H), 1.64 (m, 1H), 2.16 (m, 1H), 2.63 (s, 3H), 3.81 (s, 3H), 3.84 (s, 3H), 3.97 (m, 1H), 4.24 (d, J=11.0 Hz, 1H), 6.88 (d, J=2.8 Hz, 1H), 7.22 (dd, J=9.0, 2.9 Hz, 1H), 7.31 (m, 2H), 7.39-7.46 (m, 3H); ¹³C NMR (100 MHz, CDCl₃) δ 11.6, 11.9, 23.1, 49.5, 51.6, 55.9, 58.6, 115.9, 118.0, 125.1, 125.7, 128.5, 130.0, 130.7, 136.7, 137.1, 150.5, 155.0, 158.4, 165.6, 175.3; HRMS (ESI+) m/z calc. for C₂₃H₂₄ClN₄O₃ [M+H]⁺ 439.1531. found: 439.2110.

### Compound 14 ((+−)-methyl (S)-2-((S)-6-(4-chlorophenyl)-8-methoxy-1-methyl-4H-benzo[f][1,2,4]triazolo[4,3-a][1,4]diazepin-4-yl)butanoate)

(+−)-14 was the major product of the alkylation reaction and migrated slower than (+−)-11 on silica (CH₂Cl₂/MeOH).

Purification by flash column chromatography (gradient CH₂Cl₂/MeOH 98:2 to 96:4) afforded diastereomerically pure samples of (+−)-14. Rf 0.15 (PE₄₀₋₆₀/acetone 6:4); ¹H NMR (400 MHz, CDCl₃) δ 1.03 (t, J=7.4 Hz, 3H), 1.84 (m, 1H), 2.30 (m, 1H), 2.59 (s, 3H), 3.72 (s, 3H), 3.78-3.86 (m, 4H), 4.29 (d, J=11.0 Hz, 1H), 6.88 (d, J=2.8 Hz, 1H), 7.21 (dd, J=9.0, 2.8 Hz, 1H), 7.34 (m, 2H), 7.41 (d, J=9.0 Hz, 1H), 7.51 (m, 2H); ¹³C NMR (100 MHz, CDCl₃) δ 11.0, 12.0, 23.1, 47.3, 51.9, 56.0, 56.5, 116.1, 117.7, 125.1, 125.6, 128.6, 130.1, 130.7, 136.8, 137.2, 150.4, 155.9, 158.5, 166.3, 175.0; HRMS (ESI+) m/z calc. for C₂₃H₂₄ClN₄O₃ [M+H]⁺ 439.1531. found: 439.2122.

### Propylation of the Side Chain

A −78° C. solution of 24 (400 mg, 0.973 mmol, 1.0 eq.) in freshly distilled THF (6 mL), under Ar, was added dropwise by canulation to a −78° C. solution of KHMDS (0.5 M in toluene, 2.34 mL, 1.17 mmol, 1.2 eq.) in freshly distilled THF (14 mL), under Ar. The resulting dark solution was stirred at −78° C. for 1 h. Propyl iodide (114 μL, 1.17 mmol, 1.2 eq.) was then added dropwise, and stirring was continued for 1 h at −78° C. The temperature of the acetone bath was then gradually increased to rt over a few hours, and the mixture was stirred overnight at rt. The reaction was quenched with a few drops of AcOH and concentrated to dryness. The residue was partitioned between saturated aqueous NaHCO₃ and CHCl₃ and the aqueous phase was extracted 3 times. The combined organic layers were dried (MgSO₄) and concentrated. NMR of the crude material revealed the formation of a 1/2.2/1.18 mixture of (+−)-12, (+−)-15 and (+−)-24 respectively. Purification by flash column chromatography (CH₂Cl₂/MeOH 98:2) afforded a mixture of (+−)-12 and (+−)-15 (88 mg, 20%).

### Compound 12 ((+−)-methyl (R)-2-((S)-6-(4-chlorophenyl)-8-methoxy-1-methyl-4H-benzo[f][1,2,4]triazolo[4,3-a][1,4]diazepin-4-yl)pentanoate)

(+−)-12 was the minor product of the alkylation reaction and migrated faster than (+−)-15 on silica (CH₂Cl₂/MeOH).

Purification by flash column chromatography (CH₂Cl₂/MeOH 99:1) afforded diastereomerically pure samples of (+−)-12. Rf 0.20 (PE₄₀₋₆₀/acetone 6:4); ¹H NMR (400 MHz, CDC₁₃) δ 0.92 (t, J=7.3 Hz, 3H), 1.35 (m, 1H), 1.53 (m, 2H), 2.06 (m, 1H), 2.59 (s, 3H), 3.80 (s, 3H), 3.83 (s, 3H), 4.05 (m, 1H), 4.22 (d, J=11.1 Hz, 1H), 6.86 (d, J=2.9 Hz, 1H), 7.21 (dd, J=8.9, 2.9 Hz, 1H), 7.31 (m, 2H), 7.40 (m, 3H); ¹³C NMR (100 MHz, CDCl₃) δ 12.1, 14.0, 20.6, 32.1, 48.1, 51.6, 55.8, 59.1, 115.7, 117.9, 124.8, 126.4, 128.5, 129.9, 130.7, 136.9 (2C), 150.4, 155.1, 158.0, 165.5, 175.7; HRMS (ESI+) m/z calc. for C₂₄H₂₆ClN₄O₃ [M+H]⁺ 453.1688. found: 453.1678.

### Compound 15 ((+−)-Methyl (S)-2-((S)-6-(4-chlorophenyl)-8-methoxy-1-methyl-4H-benzo[f][1,2,4]triazolo[4,3-a][1,4]diazepin-4-yl)pentanoate)

(+−)-15 was the major product of the alkylation reaction and migrated slower than (+−)-12 on silica (CH₂Cl₂/MeOH).

Purification by flash column chromatography (CH₂Cl₂/MeOH 99:1) afforded diastereomerically pure samples of (+−)-15. Rf 0.20 (PE₄₀₋₆₀/acetone 6:4); ¹H NMR (400 MHz, CDCl₃) δ 1.01 (t, J=7.1 Hz, 3H), 1.46 (m, 2H), 1.74 (m, 1H), 2.19 (m, 1H), 2.59 (s, 3H), 3.71 (s, 3H), 3.82 (s, 3H), 3.86 (m, 1H), 4.27 (d, J=10.9 Hz, 1H), 6.88 (d, J=2.9 Hz, 1H), 7.21 (dd, J=8.9, 2.9 Hz, 1H), 7.35 (m, 2H), 7.40 (d, J=8.9 Hz, 1H), 7.52 (m, 2H); ¹³C NMR (100 MHz, CDCl₃) δ 12.1, 14.2, 20.1, 32.5, 46.2, 51.8, 55.9, 57.3, 115.8, 117.6, 125.0, 126.7, 128.5, 130.0, 130.7, 137.0, 137.2, 151.5, 156.0, 157.9, 166.1, 175.6; HRMS (ESI+) m/z calc. for C₂₄H₂₆ClN₄O₃ [M+H]⁺ 453.1688. found: 453.1673.

### Methylenecyclopropylation of the Side Chain

A −78° C. solution of 24 (400 mg, 0.973 mmol, 1.0 eq.) in freshly distilled THF (6 mL), under Ar, was added dropwise by canulation to a −78° C. solution of KHMDS (0.5 M in toluene, 2.34 mL, 1.17 mmol, 1.2 eq.) in freshly distilled THF (14 mL), under Ar. The resulting dark solution was stirred at −78° C. for 1 h. (Iodomethyl)cyclopropane (109 μL, 1.17 mmol, 1.2 eq.) was then added dropwise, and stirring was continued for 1 h at −78° C. The temperature of the acetone bath was then gradually increased to rt over a few hours, and the mixture was stirred overnight at rt. The reaction was quenched with a few drops of AcOH and concentrated to dryness. The residue was partitioned between saturated aqueous NaHCO₃ and CHCl₃ and the aqueous phase was extracted 3 times. The combined organic layers were dried (MgSO₄) and concentrated. NMR of the crude material revealed the formation of a 1/2.2/1.18 mixture of (+−)-13, (+−)-16 and (+−)-24 respectively. Purification by flash column chromatography (CH₂Cl₂/MeOH 98:2) afforded a mixture of (+−)-13 and (+−)-16 (87 mg, 19%).

### Compound 13 ((+−)-methyl (R)-2-((S)-6-(4-chlorophenyl)-8-methoxy-1-methyl-4H-benzo[f][1,2,4]triazolo[4,3-a][1,4]diazepin-4-yl)-3-cyclopropylpropanoate)

(+−)-13 was the minor product of the alkylation reaction and migrated faster than (+−)-16 on silica (CH₂Cl₂/MeOH).

Purification by flash column chromatography (CH₂Cl₂/MeOH 99:1) afforded diastereomerically pure samples of (+−)-13. Rf 0.20 (PE₄₀₋₆₀/acetone 6:4); ¹H NMR (400 MHz, CDCl₃) δ −0.05 (m, 1H), 0.12 (m, 1H), 0.42 (m, 2H), 0.80 (m, 1H), 1.65 (m, 1H), 2.14 (m, 1H), 2.59 (s, 3H), 3.80 (s, 3H), 3.85 (s, 3H), 4.21 (m, 1H), 4.27 (m, J=11.0 Hz, 1H), 6.87 (d, J=2.9 Hz, 1H), 7.21 (dd, J=9.0, 2.9 Hz, 1H), 7.32 (m, 2H), 7.38 (d, J=9.0 Hz, 1H), 7.43 (m, 2H); ¹³C NMR (100 MHz, CDCl₃) δ 3.7, 4.9, 8.7, 12.1, 34.7, 48.3, 51.7, 55.9, 58.5, 115.8, 117.9, 124.8, 128.5, 128.8, 130.0, 130.8, 136.9, 137.0, 150.2, 155.1, 158.1, 165.5, 175.5; HRMS (ESI+) m/z calc. for C₂₅H₂₆ClN₄O₃ [M+H]⁺ 465.1688. found: 465.1670.

### Compound 16 ((+−)-methyl (S)-2-((S)-6-(4-chlorophenyl)-8-methoxy-1-methyl-4H-benzo[f][1,2,4]triazolo[4,3-a][1,4]diazepin-4-yl)-3-cyclopropylpropanoate)

(+−)-16 was the major product of the alkylation reaction and migrated slower than (+−)-13 on silica (CH₂C₁₂/MeOH).

Purification by flash column chromatography (CH₂Cl₂/MeOH 99:1) afforded diastereomerically pure samples of (+−)-16. Rf 0.20 (PE₄₀₋₆₀/acetone 6:4); ¹H NMR (400 MHz, CDCl₃) δ 0.05 (m, 1H), 0.11 (m, 1H), 0.46 (m, 2H), 0.79 (m, 1H), 1.65 (m, 1H), 2.24 (m, 1H), 2.59 (s, 3H), 3.74 (s, 3H), 3.81 (s, 3H), 3.98 (m, 1H), 4.36 (d, J=11.0 Hz, 1H), 6.87 (d, J=2.9 Hz, 1H), 7.21 (dd, J=9.0, 2.9 Hz, 1H), 7.34 (m, 2H), 7.41 (d, J=9.0 Hz, 1H), 7.50 (m, 2H); ¹³C NMR (100 MHz, CDCl₃) δ 3.9, 5.0, 8.2, 12.1, 35.0, 46.6, 51.9, 55.9, 56.8, 115.7, 117.6, 125.0, 126.7, 128.6, 130.0, 130.7, 137.0, 137.1, 150.2, 156.0, 157.9, 166.0, 175.4; HRMS (ESI+) m/z calc. for C₂₅H₂₆ClN₄O₃ [M+H]⁺ 465.1688. found: 465.1678.

### Intermediate 40 (3-methyl-6-nitro-3,4-dihydroquinazolin-2(1H)-one)

To a solution of 3-methyl-3,4-dihydroquinazolin-2(1H)-one (5 g, 30.9 mmol, 1.0 equiv.) in sulphuric acid (50 mL) at 0° C. was added nitric acid (1.3 mL, 30.9 mmol, 1.0 equiv.) and the reaction mixture was stirred at 0° C. for 3 h. The solution was then poured onto ice water (200 mL) and a yellow solid crashed out of solution. The crude product was collected by filtration, and purified by flash silica column chromatography (gradient 2% to 4% MeOH/DCM) to provide the product 2 as a yellow solid (2.5 g, 39%). Rf 0.6 (6% MeOH/DCM). ¹H NMR (400 MHz, DMSO-d₆) δ=2.81 (s, 3H), 4.23 (s, 2H), 6.80 (d, J=8.3 Hz, 1H), 6.93 (dd, J=2.4, 8.3 Hz, 1H), 6.98 (d, J=2.4 Hz, 1H), 9.18 (s, 1H); HRMS [M+H]⁺ for C₉H₉N₃O₃, calcd., 208.0644. found, 208.0851.

### Intermediate 41 (6-amino-3-methyl-3,4-dihydroquinazolin-2(1H)-one)

A solution of 3-methyl-6-nitro-3,4-dihydroquinazolin-2(1H)-one (40) (1.00 g, 4.83 mmol, 1.0 equiv.) in dry methanol (100 mL) was flushed with argon before Raney nickel (200 mg) was added, and the reaction mixture was stirred at room temperature under an atmosphere of H₂ (1 atm) for 16 h. After completion of the reaction (TLC, 10% MeOH/DCM), the Raney Nickel was removed by magnetic capture and the remaining solution concentrated to provide the product as a brown solid (0.532 g, 62%) which was used without further purification. ¹H NMR (400 MHz, DMSO-d₆) δ=2.81 (s, 3H), 4.23 (s, 2H), 4.66 (s, 2H), 6.30 (d, J=2.4 Hz, 1H), 6.36 (dd, J=2.4, 8.3 Hz, 1H), 6.47 (d, J=8.3 Hz, 1H), 8.73 (s, 1H); HRMS [M+H]+ for C₉H₁₂N₃O, calcd., 178.0902. found, 178.0984.

### Compound 42 (2,4,6-trimethyl-N-(3-methyl-2-oxo-1,2,3,4-tetrahydroquinazolin-6 yl)benzenesulfonamide)

Synthesis followed same procedure as synthesis of compound 51, as described below. 0.010 g, yield 20%; ¹H NMR (400 MHz, DMSO-d6) δ=1.86 (s, 3H), 2.20 (s, 6H), 2.78 (s, 3H), 4.24 (s, 2H), 6.54 (d, J=6.8 Hz, 1H), 6.67-6.70 (m, 2H), 6.95 (s, 2H), 9.07 (s, 1H), 9.74 (s, 1H); HRMS [M+H]⁺ for C₁₈H₂₂N₃O₃S, calcd., 360.1304. found, 360.1159.

### Compound 43 (2,6-dichloro-N-(3-methyl-2-oxo-1,2,3,4-tetrahydroquinazolin-6-yl)benzenesulfonamide)

Synthesis followed same procedure as synthesis of compound 51, as described below. 0.020 g, yield 18%; ¹H NMR (400 MHz, DMSO-d6) δ=2.78 (s, 3H), 4.25 (s, 2H), 6.56 (d, J=6.4 Hz, 1H), 6.78-6.83 (m, 2H), 7.48-7.57 (m, 3H), 9.07 (s, 1H), 10.42 (s, 1H); HRMS [M+H]⁺ for C₁₅H₁₄Cl₂N₃O₃S, calcd., 386.0055. found, 386.0124.

### Compound 44 (N-(3-methyl-2-oxo-1,2,3,4-tetrahydroquinazolin-6-yl)-[1,1′-biphenyl]-2-sulfonamide)

Synthesis followed same procedure as synthesis of compound 51 as described below. 0.080 g, yield 51%; ¹H NMR (400 MHz, DMSO-d6) δ=2.79 (s, 3H), 4.23 (s, 2H), 6.55 (d, J=8.4 Hz, 1H), 6.63 (s, 1H), 6.66 (d, J=8.4 Hz, 1H), 7.20 (d, J=5 Hz, 2H), 7.24 (d, J=7.2 Hz, 1H), 7.32-7.36 (m, 3H), 7.52 (t, J=7.2 Hz, 1H), 7.58 (t, J=7.2 Hz, 1H), 7.93 (dd, J=1.4, 8.0 Hz, 1H), 9.05 (s, 1H), 9.71 (s, 1H); HRMS [M+H]⁺ for C₂₁H₂₀N₃O₃S, calcd., 394.1147. found, 394.1229.

### Compound 45 (2-chloro-6-methyl-N-(3-methyl-2-oxo-1,2,3,4-tetrahydroquinazolin-6-yl)benzenesulfonamide)

Synthesis followed same procedure as synthesis of compound 51 as described below. 0.057 g, yield 28%; ¹H NMR (400 MHz, DMSO-d6) δ=2.54 (s, 3H), 2.79 (s, 3H), 4.27 (s, 2H), 6.58 (dd, J=1.2, 8.8 Hz, 1H), 6.80 (s, 1H), 6.82 (d, J=2.0 Hz, 1H), 7.30 (dd, J=1.2, 7.4 Hz, 1H), 7.40-7.47 (m, 2H), 9.12 (s, 1H), 10.10 (s, 1H); HRMS [M+H]⁺ for C₁₆H₁₇C1N₃O₃S, calcd., 366.0601. found, 366.0680.

### Compound 46 (4-(tert-butyl)-N-(3-methyl-2-oxo-1,2,3,4-tetrahydroquinazolin-6-yl)benzenesulfonamide)

Synthesis followed same procedure as synthesis of compound 51 as described below. 0.130 g, yield 77%; ¹H NMR (400 MHz, DMSO-d6) δ=1.24 (s, 9H), 2.78 (s, 3H), 4.26 (s, 2H), 6.58 (d, J=9.3 Hz, 1H), 6.78-6.82 (m, 2H), 7.53 (d, J=8.7 Hz, 2H), 7.61 (d, J=8.7 Hz, 2H), 9.09 (s, 1H), 9.89 (s, 1H); HRMS [M+H]⁺ for C₁₉H₂₄N₃O₃S, calcd., 374.1460. found, 374.1546.

### Compound 47 (4-methoxy-N-(3-methyl-2-oxo-1,2,3,4-tetrahydroquinazolin-6-yl)benzenesulfonamide)

Synthesis followed same procedure as synthesis of compound 51, as described below. 0.095 g, yield 61%; ¹H NMR (400 MHz, DMSO-d6) δ=2.78 (s, 3H), 3.77 (s, 3H), 4.26 (s, 2H), 6.56 (d, J=8.3 Hz, 1H), 6.74-6.76 (m, 2H), 7.02 (dd, J=2.0, 8.9 Hz, 2H), 7.59 (dd, J=2.0, 8.9 Hz, 2H), 9.07 (s, 1H), 9.76 (s, 1H); HRMS [M+H]⁺ for C₁₆H₁₈N₃O₄S, calcd., 348.0940. found, 348.1047.

### Compound 48 (N-(3-methyl-2-oxo-1,2,3,4-tetrahydroquinazolin-6-yl)-[1,1′-biphenyl]-4-sulfonamide)

Synthesis followed same procedure as synthesis of compound 51, as described below. 0.064 g, yield 36%; ¹H NMR (400 MHz, DMSO-d6) δ=2.79 (s, 3H), 4.29 (s, 2H), 6.59-6.62 (m, 1H), 6.83 (d, J=2.3 Hz, 1H), 6.85 (s, 1H), 7.40-7.44 (m, 1H), 7.47-7.51 (m, 2H), 7.71 (dd, J=1.6, 7.0 Hz, 2H), 7.76 (dd, J=1.9, 8.4 Hz, 2H), 7.83 (dd, J=1.9, 8.4 Hz, 2H), 9.13 (s, 1H), 9.97 (s, 1H); HRMS [M+H]⁺ for C₂₁H₂₀N₃O₃S, calcd., 394.1147. found, 394.1226.

### Compound 49 (3,5-dimethyl-N-(3-methyl-2-oxo-1,2,3,4-tetrahydroquinazolin-6-yl)benzenesulfonamide)

Synthesis followed same procedure as synthesis of compound 51, as described below. 0.1057 g, yield 68%; ¹H NMR (400 MHz, DMSO-d6) δ=2.29 (s, 6H), 2.80 (s, 3H), 4.29 (s, 2H), 6.58-6.61 (m, 1H), 6.79 (s, 1H), 6.81 (d, J=2.2 Hz, 1H), 7.23 (td, J=0.8, 1.6 Hz, 1H), 7.31 (dd, J=0.8, 1.6 Hz, 2H), 9.12 (s, 1H), 9.85 (s, 1H); HRMS [M+H]⁺ for C₁₇H₂₀N₃O₃S, calcd., 346.1147. found, 346.1177.

### Compound 50 (4′-methoxy-N-(3-methyl-2-oxo-1,2,3,4-tetrahydroquinazolin-6-yl)-[1,1′-biphenyl]-4-sulfonamide)

Synthesis followed same procedure as synthesis of compound 51, as described below. 0.0454 g, yield 24%; ¹H NMR (400 MHz, DMSO-d6) δ=2.79 (s, 3H), 3.80 (s, 3H), 4.29 (s, 2H), 6.60 (d, J=9.2 Hz, 1H), 6.82 (s, 1H), 6.84 (s, 1H), 7.04 (d, J=8.6 Hz, 2H), 7.67 (d, J=8.7 Hz, 2H), 7.71 (d, J=8.7 Hz, 2H), 7.78 (d, J=8.7 Hz, 2H), 9.11 (s, 1H), 9.95 (s, 1H); HRMS [M+H]⁺ for C₂₂H₂₂N₃O₄S, calcd., 424.1253. found, 424.1324.

### Compound 51 (2,5-dimethoxy-N-(3-methyl-2-oxo-1,2,3,4-tetrahydroquinazolin-6-yl)benzenesulfonamide)

To a light brown suspension of 6-amino-3-methyl-3,4-dihydroquinazolin-2(1H)-one (41) (80 mg, 0.452 mmol, 1.0 equiv.) in dry dichloromethane (10 mL) under an atmosphere of argon was added pyridine (0.20 mL, 2.48 mmol, 5.9 equiv.). The addition of 2,5-dimethoxybenzene-1-sulfonyl chloride (112 mg, 0.475 mmol, 1.05 equiv.) turned the solution a deep red colour. After 3 h, the solution had turned purple and the solvent was evaporated and the residue was partitioned between ethyl acetate and aqueous 2 M HCl. The organic layer was collected, washed with water and brine, dried over magnesium sulfate, filtered, and concentrated to a residue. The residue was purified by flash column chromatography to provide the desired material (87 mg, 51%). ¹H NMR (400 MHz, DMSO-d6) δ=2.79 (s, 3H), 3.69 (s, 3H), 3.84 (s, 3H), 4.27 (s, 2H), 6.56 (d, J=9.2 Hz, 1H), 6.80 (s, 1H), 6.82 (s, 1H), 7.11 (d, J=1.2 Hz, 1H), 7.12 (s, 1H), 7.14-7.16 (m, 1H), 9.08 (s, 1H), 9.64 (s, 1H); HRMS [M+H]+ for C₁₇H₂₀N₃O₅S, calcd., 378.1045. found, 378.1124.

### Compound 52 (2-methoxy-4-methyl-N-(3-methyl-2-oxo-1,2,3,4-tetrahydroquinazolin-6-yl)benzenesulfonamide)

Synthesis followed same procedure as synthesis of compound 51, as described above. 0.0374 g, yield 23%; ¹H NMR (400 MHz, DMSO-d6) δ=2.30 (s, 3H), 2.79 (s, 3H), 3.87 (s, 3H), 4.26 (s, 2H), 6.54 (d, J=9.1 Hz, 1H), 6.79 (dt, J=1.71, 1.71, 6.17, 3H), 6.97 (s, 1H), 7.52 (d, J=8.0 Hz, 1H), 9.05 (s, 1H), 9.52 (s, 1H); HRMS [M+H]⁺ for C₁₇H₂₀N₃O₄S, calcd., 362.1096. found, 362.1172.

### Compound 53 (3-methyl-N-(3-methyl-2-oxo-1,2,3,4-tetrahydroquinazolin-6-yl)benzenesulfonamide)

Synthesis followed same procedure as synthesis of compound 51, as described above. 0.0620 g, yield 33%; ¹H NMR (400 MHz, DMSO-d6) δ=2.32 (s, 3H), 2.79 (s, 3H), 4.27 (s, 2H), 6.58 (d, J=9.1 Hz, 1H), 6.76-6.80 (m, 2H), 7.39 (s, 1H), 7.40 (s, 1H), 7.44-7.48 (m, 1H), 7.51 (s, 1H), 9.10 (s, 1H), 9.89 (s, 1H); HRMS [M+H]⁺ for C₁₆H₁₈N₃O₃S, calcd., 332.0991. found, 332.1069.

### Compound 54 (2,4-dimethoxy-N-(3-methyl-2-oxo-1,2,3,4-tetrahydroquinazolin-6-yl)benzenesulfonamide)

Synthesis followed same procedure as synthesis of compound 51, as described above. 0.1057 g, yield 68%; ¹H NMR (400 MHz, DMSO-d6) δ=2.79 (s, 3H), 3.78 (s, 3H), 3.88 (s, 3H), 4.26 (s, 2H), 6.52-6.55 (m, 2H), 6.63 (d, J=2.3 Hz, 1H), 6.78 (s, 1H), 6.79 (s, 1H), 7.56 (d, J=8.8 Hz, 1H), 9.05 (s, 1H), 9.49 (s, 1H); HRMS [M+H]⁺ for C₁₇H₂₀N₃O₅S, calcd., 378.1045. found, 378.1118.

### General Procedure for the Alkylation in α-Position

I-Bet-OMe (200 mg, 487 μmol, 1 eq.) or 9-I-Bet-OMe (200 mg, 487 μmol, 1 eq.) were dissolved in anhydrous tetrahydrofuran (5 ml in the case of 1-Bet-OMe and 10 ml in the case of 9-I-Bet-OMe). This solution was then added drop wise to a solution of Potassium bis(trimethylsilyl)amide (1.17 ml of a 0.5 M solution in toluene, 584 μmol, 1.2 eq.) in tetrahydrofuran at −80° C. under an atmosphere of nitrogen. After 1 h at this temperature the corresponding alkyl iodide (584 μmol, 1.2 eq.) was added drop wise. The reaction mixture was warmed to 25° C. over 18 h and a few drops of acetic acid were then added to quench the reaction. The solvent was removed in vacuo and the residue purified by flash column chromatography using a linear gradient from 10% to 60% acetone in heptane. For isomerizing the intermediate together with sodium methoxide (10 eq.) was dissolved in methanol (2 ml) and heated to 120° C. for 40 min in a microwave reactor. The reaction mixture was acidified with aqueous hydrochloric acid (1 M), diluted with water and extracted three times with dichloromethane. The combined organic phases were dried over manganese sulfate and evaporated to dryness. The diastereoisomers were separated by reversed phase column chromatography. Compounds I-Bet-OMe [12], I-Bet [12], 9-I-Bet-OMe [14] and 9-I-Bet2 [14] were prepared according to literature procedures.

### (+−)methyl (R)-2-((S)-6-(4-chlorophenyl)-8-methoxy-1-methyl-4H-benzo[f][1,2,4]triazolo[4,3-a][1,4]diazepin-4-yl)pent-4-enoate (AL)

Yield: 32.3 mg (15%); ¹H-NMR (CDCl₃, 500 MHz) δ 2.40-2.46 (m, 1H), 2.60 (s, 3H), 2.88-2.93 (m, 1H), 3.80 (s, 3H), 3.81 (s, 3H), 4.12-4.16 (m, 1H), 4.27 (d, 1H, J(H,H)=11.0 Hz), 4.99-5.06 (m, 2H), 5.82-5.90 (m, 1H), 6.87 (d, 1H, J(H,H)=2.90 Hz), 7.21 (dd, 1H, J(H,H)=2.90 Hz, J(H,H)=8.90 Hz), 7.30-7.33 (m, 2H), 7.39-7.43 (m, 3H); ¹³C-NMR (CDCl3, 126 MHz) δ 12.1, 34.2, 47.8, 51.5, 55.8, 58.4, 115.8, 117.2, 117.9, 124.8, 126.4, 128.5, 129.9, 130.7, 134.4, 137.0, 150.4, 154.9, 158.0, 165.5, 174.7; HRMS m/z calc. for C₂₄H₂₄ClN₄O₃ [M+H]⁺ 451.1531. found 451.1523.

### (+−) methyl (R)-2-((S)-6-(4-chlorophenyl)-9-methoxy-1-methyl-4H-benzo[f][1,2,4]triazolo[4,3-a][1,4]diazepin-4-yl)propanoate (9-ME)

Yield: 35.9 mg (17%); ¹H-NMR (CDCl₃, 400 MHz) δ 1.49 (d, 3H, J(H,H)=6.64 Hz), 2.64 (s, 3H), 3.82 (s, 3H), 3.95 (s, 3H), 4.05-4.11 (m, 1H), 4.23 (d, 1H, J(H,H)=11.04 Hz), 6.94 (s, 1H), 6.98 (d, 1H, J(H,H)=8.96 Hz), 7.31 (d, 2H, J(H,H)=7.40 Hz), 7.35-7.39 (m, 3H); ¹³C-NMR (CDCl₃, 101 MHz) δ 12.3, 15.3, 42.4, 51.8, 55.9, 59.6, 109.4, 112.7, 121.4, 128.4, 130.8, 133.4, 134.7, 136.7, 137.4, 150.2, 154.9, 161.6, 165.7, 176.1; HRMS m/z calc. for C₂₂H₂₂ClN₄O₃ [M+H⁺] 425.1375. found 425.1381.

### (+−) methyl (R)-2-((S)-6-(4-chlorophenyl)-9-methoxy-1-methyl-4H-benzo[f][1,2,4]triazolo[4,3-a][1,4]diazepin-4-yl)butanoate (9-ET)

Yield: 25.6 mg (12%); ¹H-NMR (CDCl₃, 400 MHz) δ 1.01 (t, 3H, J(H,H)=7.28 Hz), 1.57-1.68 (m, 1H), 2.12-2.21 (m, 1H), 2.63 (s, 3H), 3.84 (s, 3H) 3.95 (s, 3H), 4.00 (dd, 1H, J(H,H)=2.80 Hz, J(H,H)=11.2 Hz), 4.23 (d, 1H, J(H,H)=10.9 Hz), 6.94 (s, 1H) 6.98 (d, 1H, J(H,H)=8.48 Hz), 7.30 (d, 2H, J(H,H)=7.68 Hz), 7.34-7.38 (m, 3H); ¹³C-NMR (CDCl₃, 101 MHz) δ 11.6, 12.4, 23.2, 49.7, 51.6, 55.9, 58.8, 109.4, 112.7, 121.4, 128.4, 130.8, 133.4, 134.7, 136.7, 137.4, 150.2, 155.1, 161.6, 165.8, 175.5; HRMS m/z calc. for C₂₃H₂₄ClN₄O₃ [M+H⁺] 439.1531. found 439.1513.

### (+−)methyl (R)-2-((S)-6-(4-chlorophenyl)-9-methoxy-1-methyl-4H-benzo[f][1,2,4]triazolo[4,3-a][1,4]diazepin-4-yl)pent-4-enoate (9-AL)

Yield: 27.3 mg (12%); ¹H-NMR (CDCl₃, 400 MHz) δ 2.38-2.46 (m, 1H), 2.64 (s, 3H), 2.88-2.94 (m, 1H), 3.80 (s, 3H), 3.95 (s, 3H), 4.11-4.17 (m, 1H), 4.27 (d, 1H, J(H,H)=10.9 Hz), 4.99-5.07 (m, 2H), 5.82-5.92 (m, 1H), 6.93 (d, 1H, J(H,H)=2.44 Hz), 6.98 (dd, 1H, J(H,H)=2.48 Hz, J(H,H)=8.80 Hz), 7.29-7.32 (m, 2H), 7.34-7.39 (m, 3H); ¹³C-NMR (CDCl₃, 101 MHz) δ 12.4, 34.2, 47.9, 51.6, 55.9, 58.3, 109.4, 112.7, 117.2, 121.3, 128.4, 130.8, 133.4, 134.5, 134.7, 136.8, 137.3, 150.3, 154.8, 161.6, 165.9, 174.7; HRMS m/z calc. for C₂₄H₂₄ClN₄O₃ [M+H⁺] 451.1531. found 451.1540.

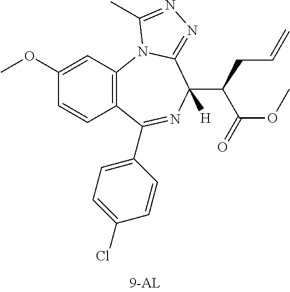 [SMILES: C=CC[C@@H](C(=O)OC)[C@@H]1N=C(c2ccc(Cl)cc2)c2ccc(OC)cc2-n2c(C)nnc21]

### General Procedure for Amide Formation

The mixture of diasteroisomers of the ester compounds (100 μmol, 1 eq.) were hydrolyzed in methanol (0.5 ml) and aqueous sodium hydroxide (0.5 ml, 1 M in water) by heating to 100° C. for 30 min in a microwave oven. After quenching with aqueous hydrochloric acid (1 M) the reaction mixture was extracted three times with dichloromethane. The combined organic phases were dried over manganese sulfate and evaporated to dryness. To this end, the obtained free carboxylic acid was dissolved in dichloromethane, the corresponding amine (150 μmol, 1.5 eq.), HATU (57.0 mg, 150 μmol, 1.5 eq.) and N,N-diispropylethylamine (69.9 μl, 400 μmol, 4 eq.) were added and the reaction mixture stirred at 25° C. for 2 h. The solvent was removed and the residue subject to flash column chromatography before the diastereoisomers were separated by reversed phase column chromatography.

### (+−) (R)-2-((S)-6-(4-chlorophenyl)-8-methoxy-1-methyl-4H-benzo[f][1,2,4]triazolo[4,3-a][1,4]diazepin-4-yl)-N-ethylpropanamide (ME-Am₁)

Yield: 13.2 mg (30%); ¹H-NMR (CDCl₃, 500 MHz) δ 1.25 (t, 3H, J(H,H)=7.25 Hz), 1.44 (d, 3H, J(H,H)=6.75 Hz), 2.59 (s, 3H), 3.33-3.50 (m, 2H), 3.63-3.69 (m, 1H), 3.79 (s, 3H), 4.24 (d, 1H, J(H,H)=9.80 Hz), 6.25 (t, 1H, J(H,H)=5.49 Hz), 6.85 (d, 1H, J(H,H)=2.90 Hz), 7.20 (dd, 1H, J(H,H)=2.90 Hz, J(H,H)=8.90 Hz), 7.29-7.32 (m, 2H), 7.39 (d, 1H, J(H,H)=8.85 Hz), 7.43-7.46 (m, 2H); ¹³C-NMR (CDCl₃, 126 MHz) δ 12.1, 15.0, 15.6, 34.4, 43.6, 55.8, 59.7, 115.6, 118.1, 124.8, 126.4, 128.4, 130.0, 136.9, 137.1, 150.3, 155.6, 158.0, 165.5, 174.4; HRMS m/z calc. for C₂₃H₂₅ClN₅O₂ [M+H⁺] 438.1691. found 438.1675.

### (+−) (R)-2-((S)-6-(4-chlorophenyl)-8-methoxy-1-methyl-4H-benzo[f][1,2,4]triazolo[4,3-a][1,4]diazepin-4-yl)-N-ethylbutanamide (ET-Am₁)

Yield: 11.7 mg (26%); ¹H-NMR (CDCl₃, 500 MHz) δ 1.03 (t, 3H, J(H,H)=7.35 Hz), 1.27 (t, 3H, J(H,H)=7.25 Hz), 1.64-1.74 (m, 1H), 2.01-2.09 (m, 1H), 2.59 (s, 3H), 3.42-3.49 (m, 3H), 3.79 (s, 3H), 4.23 (d, 1H, J(H,H)=10.0 Hz), 6.17 (s, 1H), 6.85 (d, 1H, J(H,H)=2.85 Hz), 7.20 (dd, 1H, J(H,H)=2.90 Hz, J(H,H)=10.4 Hz), 7.30-7.21 (m, 2H), 7.38 (d, 1H, J(H,H)=8.90 Hz), 7.42-7.45 (m, 2H); ¹³C-NMR (CDCl₃, 126 MHz) δ 11.9, 12.1, 15.2, 22.9, 34.4, 51.3, 55.8, 59.0, 115.6, 118.1, 124.8, 126.5, 128.4, 130.0, 130.8, 136.9, 137.1, 150.3, 155.7, 158.0, 165.4, 173.5; HRMS m/z calc. for C₂₄H₂₆ClN₅O₂ [M+H⁺] 452.1848. found 452.1839.

### (+−) (R)-2-((S)-6-(4-chlorophenyl)-8-methoxy-1-methyl-4H-benzo[f][1,2,4]triazolo[4,3-a][1,4]diazepin-4-yl)-N,N-diethylpropanamide (ME-Am₂)

Yield: 17.3 mg (37%); ¹H-NMR (CDCl₃, 400 MHz) δ 1.24 (t, 3H, J(H,H)=7.24 Hz), 1.33 (t, 3H, J(H,H)=6.92 Hz), 1.41 (d, 3H, J(H,H)=6.64 Hz), 2.65 (s, 3H), 3.37-3.45 (m, 1H), 3.50-3.74 (m, 3H), 3.80 (s, 3H), 4.21-4.29 (m, 1H), 4.40 (d, 1H, J(H,H)=10.6 Hz), 6.88 (s, 1H), 7.21-7.29 (m, 1H), 7.30 (d, 2H), 7.41-7.46 (m, 3H); ¹³C-NMR (CDCl₃, 101 MHz) δ 11.8, 13.4, 15.0, 15.6, 38.0, 40.7, 42.5, 55.9, 60.4, 115.7, 118.1, 125.0, 125.8, 128.3, 130.0, 130.8, 136.9, 137.0, 150.7, 155.8, 158.3, 165.1, 174.3; HRMS m/z calc. for C₂₅H₂₉ClN₅O₂ [M+H⁺] 466.2004. found 466.1997.

### (+−) (R)-2-((S)-6-(4-chlorophenyl)-8-methoxy-1-methyl-4H-benzo[f][1,2,4]triazolo[4,3-a][1,4]diazepin-4-yl)-N,N-diethylbutanamide (ET-Am₂)

Yield: 15.8 mg (33%); ¹H-NMR (CDCl₃, 400 MHz) δ 1.00 (t, 3H, J(H,H)=7.28 Hz), 1.26 (t, 3H, J(H,H)=7.00 Hz), 1.33 (t, 3H, J(H,H)=6.84 Hz), 1.65-1.74 (m, 1H), 2.04-2.11 (m, 1H), 2.62 (s, 3H), 3.50-3.76 (m, 4H), 3.80 (s, 3H), 4.11-4.23 (m, 1H), 4.31 (d, 1H, J(H,H)=10.5 Hz), 6.86 (s, 1H), 7.19-7.22 (m, 1H), 7.29 (d, 2H, J(H,H)=7.64 Hz), 7.40-7.43 (m, 3H); ¹³C-NMR (CDCl₃, 101 MHz) δ 11.6, 11.9, 13.4, 14.8, 40.8, 42.5, 44.4, 55.9, 59.9, 115.7, 118.1, 124.9, 126.0, 128.3, 129.9, 130.8, 136.8, 137.1, 150.6, 155.9, 158.2, 165.2, 173.5; HRMS m/z calc. for C₂₆H₃₁ClN₅O₂ [M+H⁺] 480.2161. found 480.2171.

### (+−) (R)-2-((S)-6-(4-chlorophenyl)-9-methoxy-1-methyl-4H-benzo[f][1,2,4]triazolo[4,3-a][1,4]diazepin-4-yl)-N-ethylpropanamide (9-ME-Am₁)

Yield: 12.6 mg (29%); ¹H-NMR (CDCl₃, 400 MHz) δ 1.24 (t, 3H, J(H,H)=7.20 Hz), 1.44 (d, 3H, J(H,H)=6.56 Hz), 2.62 (s, 3H), 3.32-3.50 (m, 2H), 3.62-3.70 (m, 1H), 3.93 (s, 3H), 4.23 (d, 1H, J(H,H)=9.64 Hz), 6.28 (s, 1H), 6.93-6.97 (m, 2H), 7.28-7.34 (m, 3H), 7.40 (d, 2H, J(H,H)=7.62 Hz); ¹³C-NMR (CDCl₃, 101 MHz) δ 12.3, 15.0, 15.6, 34.4, 43.6, 55.9, 59.6, 109.4, 112.7, 121.4, 128.3, 130.8, 133.4, 134.7, 136.7, 137.5, 150.2, 155.5, 161.5, 165.8, 174.4; HRMS m/z calc. for C₂₃H₂₅ClN₅O₂ [M+H⁺] 438.1691. found 438.1684.

### (+−) (R)-2-((S)-6-(4-chlorophenyl)-9-methoxy-1-methyl-4H-benzo[f][1,2,4]triazolo[4,3-a][1,4]diazepin-4-yl)-N-ethylbutanamide (9-ET-Am₁)

Yield: 9.5 mg (21%); ¹H-NMR (CDCl₃, 400 MHz) δ 1.03 (t, 3H, J(H,H)=7.24 Hz), 1.24 (t, 3H, J(H,H)=7.24 Hz), 1.70-1.78 (m, 1H), 1.96-2.06 (m, 1H), 2.64 (s, 3H), 3.41-3.48 (m, 3H), 3.94 (s, 3H), 4.26 (d, 1H, J(H,H)=9.32 Hz), 6.43 (s, 1H), 6.97-6.99 (m, 2H), 7.31-7.34 (m, 3H), 7.42 (d, 2H, J(H,H)=7.94 Hz); ¹³C-NMR (CDCl₃, 101 MHz) δ 11.9, 12.2, 15.0, 23.2, 34.4, 50.9, 56.0, 58.4, 109.4, 113.3, 121.2, 128.4, 130.9, 133.5, 134.4, 137.0, 137.3, 150.5, 155.5, 161.8, 173.7; HRMS m/z calc. for C₂₄H₂₇ClN₅O₂ [M+H⁺] 452.1848. found 452.1855.

### (+−) (R)-2-((S)-6-(4-chlorophenyl)-8-methoxy-1-methyl-4H-benzo[f][1,2,4]triazolo[4,3-a][1,4]diazepin-4-yl)-N-ethylpent-4-enamide (AL-Am₁)

Yield: 14.7 mg (32%); ¹H-NMR (CDCl₃, 400 MHz) δ 1.19 (t, 3H, J(H,H)=7.20 Hz), 2.48-2.56 (m, 1H), 2.62 (s, 3H), 2.70-2.76 (m, 1H), 3.32-3.44 (m, 2H), 3.52-3.58 (m, 1H), 3.80 (s, 3H), 4.32 (d, 1H, J(H,H)=8.48 Hz), 5.02 (d, 1H, J(H,H)=10.3 Hz), 5.10 (d, 1H, J(H,H)=17.0 Hz), 5.82-5.92 (m, 1H), 6.60 (s, 1H), 6.86 (d, 1H, J(H,H)=2.84 Hz), 7.23 (dd, 1H, J(H,H)=2.84 Hz, J(H,H)=8.96 Hz), 7.33-7.35 (m, 2H), 7.44-7.48 (m, 3H); ¹³C-NMR (CDCl₃, 101 MHz) δ 11.8, 14.9, 34.4, 48.5, 55.9, 57.7, 116.1, 117.5, 118.1, 125.2, 125.6, 128.5, 129.9, 130.9, 134.8, 136.8, 137.2, 150.8, 155.3, 158.4, 166.4, 172.9; HRMS m/z calc. for C₂₅H₂₇ClN₅O₂ [M+H⁺] 464.1848. found 464.1840.

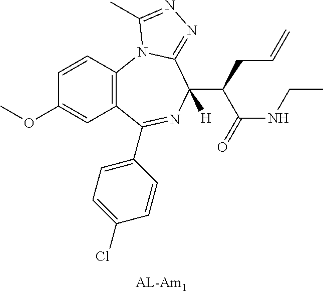 [SMILES: C=CC[C@@H](C(=O)NCC)[C@@H]1N=C(c2ccc(Cl)cc2)c2cc(OC)ccc2-n2c(C)nnc21]

Fluorescence Recovery after Photobleaching (FRAP)

Fluorescence recovery after photobleaching (FRAP) experiments were performed in human osteosarcoma U2OS cells transfected with mammalian expression constructs encoding wild type and mutant GFP chimeras of Brd4. Cells were cultured in DMEM (Gibco) supplemented with fetal bovine serum, Penicillin/Streptamycin and L-Glutamine. Cells were seeded into glass bottom dishes (Willco) to about 40% confluency and transfected with the constructs using Effectene (QIAGEN) at least 18 h before the experiment. Treatment of cells with 1 μM compounds (in DMSO) was performed 12-15 h before the experiment. Cells without compound treatment were treated with DMSO as a vehicle control at least 15 h before the experiment. DMEM was exchanged for CO₂-independent phenol red-free media (Gibco) for the experiment. FRAP studies were performed using a DeltaVision Core mounted on an Olympus IX70 stand with a 60×1.4NA plan apo objective lens equipped with a heated chamber set to 37° C. and a Quantifiable Laser Module (QLM) with 10 mW 488 nm solid state laser delivering a diffraction limited spot to the centre field of view. A 490/20 nm excitation and a 528/38 nm emission filter were used. A spot was bleached with a single pulse at 100% laser power for 0.2 s and recovery images were acquired using a coolsnap HQ camera with a 2×2 bin at 0.05 s exposure. Three pre event images were taken, as well as 32 post event images over the course of 20 s in total, the first of which was acquired 0.02 s after the bleach event. FRAP data was analysed using the SoftWorX software. It was fitted to a 2-dimensional recovery curve using the method of Axelrod as implemented within the software and half-times of recovery were calculated.

### REFERENCES

1. GlaxoSmithKline. A study to investigate the safety, pharmacokinetics, pharmacodynamics, and clinical activity of gsk525762 in subjects with nut midline carcinoma (nmc) and other cancers. Technical report, National Institute of Health, Clinicaltrials.gov identifier NCT01587703.
2. Delmore, J. E., Issa, G. C., Lemieux, M. E., Rahl, P. B., Shi, J., Jacobs, H. M., Kastritis, E., Gilpatrick, T., Paranal, R. M., Qi, J., Chesi, M., Schinzel, A. C., McKeown, M. R., Heffernan, T. P., Vakoc, C. R., Bergsagel, P. L., Ghobrial, I. M., Richardson, P. G., Young, R. A., Hahn, W. C., Anderson, K. C., Kung, A. L., Bradner, J. E. and Mitsiades, C. S., Bet bromodo-main inhibition as a therapeutic strategy to target c-myc. Cell, 146(6):904-17, September 2011.
3. Dawson, M. A., Prinjha, R. K., Dittmann, A., Giotopoulos, G., Bantscheff, M., Chan, W-I., Robson, S. C., Chung, C-w., Hopf, C., Savitski, M. M., Huthmacher, C., Gudgin, E., Lugo, D., Beinke, S., Chapman, T. D., Roberts, E. J., Soden, P. E., Auger, K. R., Mirguet, O., Doehner, K., Delwel, R., Burnett, A. K., Jeffrey, P., Drewes, G., Lee, K., Huntly, B. J. P. and Kouzarides, T., Inhibition of BET recruitment to chromatin as an effective treatment for MLL-fusion leukaemia. Nature, 478(7370):529-533, October 2011.
4. Zuber, J., Shi, J., Wang, E., Rappaport, A. R., Herrmann, H., Sison, E. A., Magoon, D., Qi, J., Blatt, K., Wunderlich, M., Taylor, M. J., Johns, C., Chicas, A., Mulloy, J. C., Kogan, S. C., Brown, P., Valent, P., Bradner, J. E., Lowe, S. W. and Vakoc, C. R., RNAi screen identifies Brd4 as a therapeutic target in acute myeloid leukaemia. Nature, 478(7370):524-528, August 2011.
5. Picaud S, et al, PFI-1, a Highly Selective Protein Interaction Inhibitor, Targeting BET Bromodomains, Cancer Res Jun. 1, 2013 73; 3336.
6. Dhalluin, C., Carlson, J. E., Zeng, L., He, C., Aggarwal, A. K., Zhou, M.-M., Structure and ligand of a histone acetyltransferase bromodomain. Nature 1999, 399 (6735), 491-496.
7. Owen, D. J., Ornaghi, P., Yang, J. C., Lowe, N., Evans, P. R., Ballario, P., Neuhaus, D., Filetici, P., Travers, A. A., The structural basis for the recognition of acetylated histone H4 by the bromodomain of histone acetyltransferase Gcn5p. EMBO 2000, 19 (22), 6141-6149.
8. Mujtaba, S., He, Y., Zeng, L., Farooq, A., Carlson, J. E., Ott, M., Verdin, E., Zhou, M.-M., Structural Basis of Lysine-Acetylated HIV-1 Tat Recognition by PCAF Bromodomain. Mol. Cell 2002, 9 (3), 575-586.
9. Friesner, R. A., Banks, J. L., Murphy, R. B., Halgren, T. A., Klicic, J. J., Mainz, D. T., Repasky, M. P., Knoll, E. H., Shaw, D. E., Shelley, M., Perry, J. K., Francis, P., Shenkin, P. S., Glide: A new approach for rapid, accurate docking and scoring. 1. Method and Assessment of docking accuracy. J. Med. Chem. 2004, 47, 1739-1749.
10. P. Filippakopoulos et al., Selective inhibition of BET bromodomains. Nature 2010, 468, 1067-1073.
11. Niesen, F. H., Berglund, H. and Vedadi, M., The use of differential scanning fluorimetry to detect ligand interactions that promote protein stability. Nat Protoc, 2(9):2212-21, 2007.
12. Chung, C. W., Coste, H., White, J. H., Mirguet, O. J., Wilde, R. L., Gosmini, C. Delves, Magny, S. M., Woodward, R., Hughes, S. A., Boursier, E. V., Flynn, H., Bouillot, A. M., Bamborough, P., Brusq, J. M., Gellibert, F. J., Jones, E. J., Riou, A. M., Homes, P., Martin, S. L., Uings, I. J., Toum, J., Clement, C. A., Boullay, A. B., Grimley, R. L., Blandel, F. M., Prinjha, R. K., Lee, K., Kirilovsky, J. and Nicodeme E., Discovery and characterization of small molecule inhibitors of the BET family bromodomains. J. Med. Chem. 54, 3827-3838, 2011.
13. Axelrod, D., Koppel, D. E., Schlessinger, J., Elson, E., Webb, W. W., Mobility measurement by analysis of fluorescence photobleaching recovery kinetics. Biophys. J., 1976, 16, 1055-1069.
14. O. Mirguet et al., Discovery of epigenetic regulator I-BET762: lead optimization to afford a clinical candidate inhibitor of the BET bromodomains. J. Med. Chem. 56, 7501-7515, 2013.
15. Baud., M. G. J., et al., A bump-and-hole approach to engineer controlled selectivity of BET bromodomain chemical probes. Science, 346, 6209, 638-641, 2014.

1. A method of selectively inhibiting a bromodomain in a protein in the presence of a plurality of other wild type bromodomains, the method comprising the steps of:
introducing a functionally silent mutation into a bromodomain in a protein in the presence of a plurality of other wild type bromodomains; and
selectively inhibiting the mutated bromodomain.

2. A method of identifying the physiological function of a bromodomain in a protein, the method comprising the steps of:
introducing a functionally silent mutation into one bromodomain in a protein in the presence of a plurality of other wild type bromodomains;
selectively inhibiting the mutated bromodomain; and
evaluating the effect of the inhibition.

3. The method according to claim 1, wherein the step of selectively inhibiting the mutated bromodomain includes addition of a compound which specifically binds the mutated bromodomain.

4. The method according to claim 1, wherein the protein is a bromo and extra-terminal (BET) protein.

5. The method according to claim 1, wherein the protein is selected from the group consisting of Brd2(1), Brd2(2), Brd3(1), Brd3(2), Brd4(1), Brd4(2), Brdt(1) and Brdt(2).

6. (canceled)

7. The method according to claim 1, wherein the functionally silent mutation is introduced by site directed mutagenesis.

8. The method according to claim 1, wherein the functionally silent mutation is introduced at an amino acid position which is conserved between bromodomains.

9. The method according to claim 8, wherein the functionally silent mutation is introduced at a conserved position equivalent to Leu94 or Met149 in Brd4(1).

10. The method according to claim 8, wherein the functionally silent mutation is generated by replacement of an amino acid with alanine, valine or isoleucine.

11. A method according to claim 1, wherein inhibition of the mutated bromodomain is at least 30 fold greater than inhibition of the wild type bromodomain.

12. The method according to claim 3, wherein the compound has the formula (I): 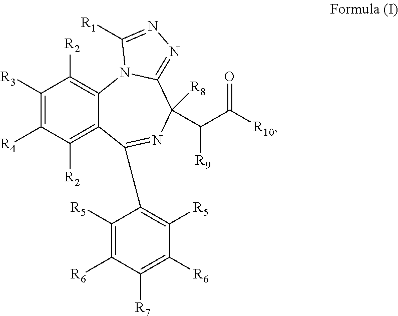
wherein
each one of R₁, R₂, R₃, R₄ and R₈ are independently hydrogen, a C1-6 linear, branched or substituted alkyl, alkenyl, alkynyl or alkoxy group;
each one of R₅, R₆ and R₇ are independently: hydrogen, halogen, NR₁₁R₁₂ or a C1-6 linear, branched or substituted alkyl, alkenyl, alkynyl group;
any two of R₄, R₅ and R₆, together with the atoms to which they are attached optionally are joined to form an optionally substituted C1-6 cycloalkyl, heterocyclic, aromatic or heteroaromatic moiety;
R₁₁ and R₁₂ are independently hydrogen or C1-6 linear, branched or substituted alkyl, alkenyl, alkynyl group;
R₉ is hydrogen, or C1-6 linear, or branched alkyl, alkenyl or alkynyl, optionally substituted by one or more amine or hydroxy groups; and
R₁₀ is R₁₃, OR₁₃, NHR₁₃ or NR₁₃R₁₃, or an optionally substituted C1-6 cycloalkyl, heterocyclic, aromatic or heteroaromatic moiety, wherein R₁₃ is a C1-6 linear, or branched alkyl, alkenyl or alkynyl group;
with the proviso that where R₄ is methoxy, at least one of R₂, R₃, R₅, R₆, R₈ or R₉ is not hydrogen.

13. The method according to claim 12, wherein the compound has the formula (II) 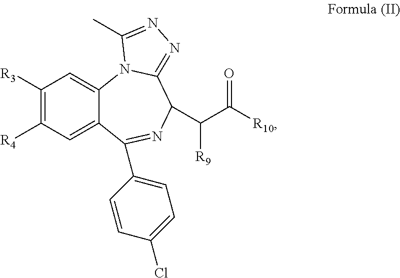
wherein
one or both of R₃ and R₄ are alkoxy groups;
R₉ is hydrogen, or C1-6 linear or branched alkyl, alkenyl or alkynyl, optionally substituted by one or more amine or hydroxy groups; and
R₁₀ is R₁₃, OR₁₃, NHR₁₃ or NR₁₃R₁₃, or an optionally substituted C1-6 cycloalkyl, heterocyclic, aromatic or heteroaromatic moiety. R₁₃ is a C1-6 linear or branched alkyl, alkenyl or alkynyl group
or wherein the compound has the formula (III) 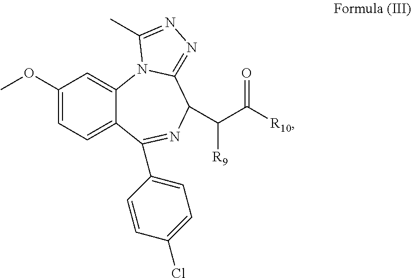
wherein R₉ is hydrogen, or C1-6 linear or branched alkyl, alkenyl or alkynyl, optionally substituted by one or more amine or hydroxy groups; and
R₁₀ is R₁₃, OR₁₃, NHR₁₃ or NR₁₃R₁₃, or an optionally substituted C1-6 cycloalkyl, heterocyclic, aromatic or heteroaromatic moiety, wherein R₁₃ is a C1-6 linear or branched alkyl, alkenyl or alkynyl group
or wherein the compound has the formula (IV) 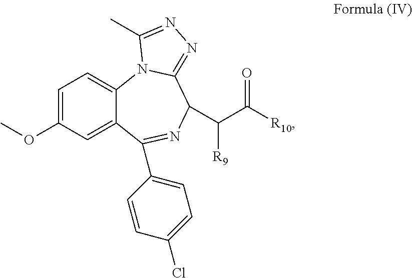
wherein R₉ is hydrogen, or C1-6 linear or branched alkyl, alkenyl or alkynyl, optionally substituted by one or more amine or hydroxy groups; and
R₁₀ is R₁₃, OR₁₃, NHR₁₃ or NR₁₃R₁₃, or an optionally substituted C1-6 cycloalkyl, heterocyclic, aromatic or heteroaromatic moiety, wherein R₁₃ is a C1-6 linear or branched alkyl, alkenyl or alkynyl group.

14. (canceled)

15. (canceled)

16. (canceled)

17. The method according to claim 13, wherein R₉ is a C1-4 linear, branched or cycloalkyl group and R₁₀ is OR₁₃, and further wherein R₁₃ is a C1-6 linear or branched alkyl, alkenyl or alkynyl group.

18. (canceled)

19. (canceled)

20. The method according to claim 12, wherein the compound has the formula (V): 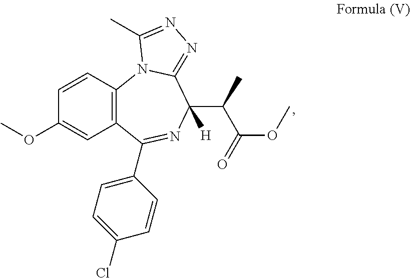
or wherein the compound has the formula (VI): 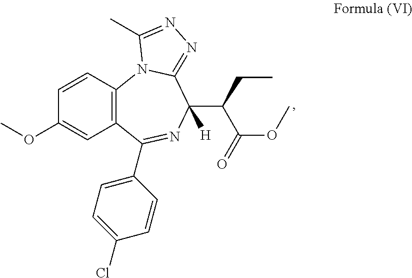
or wherein the compound has the formula (VII): 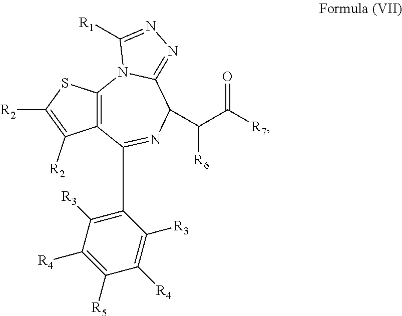
wherein R₁, R₂, R₃, R₄ and R₅ are independently hydrogen, a halogen or a C1-6 linear, branched or substituted alkyl, alkenyl or alkynyl group;
R₆ is a C1-6 linear or branched alkyl, alkenyl or alkynyl group, optionally substituted by one or more amine or hydroxy groups;
R₇ is OH, OR₈, NHR₈ or NR₈R₉; and
R₈ and R₉ is a C1-6 linear, branched or substituted alkyl, alkenyl or alkynyl group.

21. (canceled)

22. (canceled)

23. The method according to claim 20, wherein R₈ and R₉, together with the atom to which they are attached are fused to form a C1-6, heterocyclic, heteroaromatic, substituted heterocyclic or substituted heteroaromatic ring.

24. The method according to claim 20, wherein R₂ is a methyl group.

25. The method according to claim 20, wherein R₇ is OR₈ and R₈ is methyl or tertiary-butyl (t-butyl).

26. The method according to claim 20, wherein R₇ is NHR₈ and R₈ is ethyl.

27. (canceled)

28. (canceled)

29. (canceled)
